SEQUENCE LISTING (82 sequences)

SEQ ID NO: 1 - DNA, 4098 bp, Xanthomonas oryzae
atggatcccattcgtccgcgcaggccaagtcctgcccgcgagcttctgcccggaccccaaccggatagggttcagccgactgcagatcgtggggtgtctgcgcctgctggcagccctctggatggcttgcccgctcggcggacggtgtcccggacccggctgccatctccccctgcgccctcacctgcgttctcggcgggcagcttcagcgatctgctccgtccgttcgatccgtcgcttcttgatacatcgcttcttgattcgatgcctgccgtcggcacgccgcatacagcggctgccccagcagagtgggatgaggcgcaatcggctctgcgtgcagccgatgacccgccacccaccgtgcgtgtcgctgtcactgccgcgcggccgccgcgcgccaagccggccccgcgacggcgtgctgcgcaaccctccgacgcttcgccggccgcgcaggtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggcacaggtggtggccatcgccagcaataacggtggcaagcaggcgctggagacggtgcagcggctgttgccggtgctgtgccaggcccatggcctgaccccggcgcaggtggtggccatcgccagccacgatggcggcaagcaggcactggagacgatgcagcggctgttgccggtgctgtgccaggcccatggcctgcccccggaccaggtggtggccatcgccagcaatattggcggcaagcaggcgctggagacggtgcagcggctgttgccggtgctctgccaggcccatggcctgaccccggaccaggtggtggccatcgccagccatggcggcggcaagcaggcgctggagacggtgcagcggctgttgccggtgctctgccaggcccatggcctgaccccggaccaggtggtggccatcgccagccacgatggcggcaagcaggcgctggagacggtgcagcggctgttgccggtgctgtgccaggcccatggcctgaccccggaccaggtggtggccatcgccagcaatggcggcggcaagcaggcgctggagacggtgcagcggctgttgccggtgctgtgccaggcccatggtctgaccccggaccaggtggtggccatcgccagcaatggcggcaagcaggcgctggagacggtgcagcggctgttgccggtgctgtgccaggcccatggcctgaccccggaccaggtggtggccatcgccagccacgatggcggcaagcaggcgctggagacggtgcagcggctgttgccggtgctgtgccagacccatggtctgaccccggcgcaggtggtggccatcgccagccacgatggcggcaagcaggcgctggagacggtgcagcagctgttgccggtgctgtgccaggcccatggcctgaccccggaccaggtggtggccatcgccagcaatattggcggcaagcaggcgctagcgacggtgcagcggctgttgccggtgctgtgccaagcccatggcctgaccccggaccaggtggtggccatcgccagcaatggcggcggcaagcaggcgctggagacggtgcagcggctgttgccggtgctgtgccaggcccatggcctgaccccggaccaggtggtggccatcgccagcaatggcggcggcaagcaggcgctggagacggtgcagcggctgttgccggtgctgtgccaggcccatggtctgacccaggtgcaggtggtggccatcgccagcaatattggcggcaagcaggcgctggagacggtgcagcggctgttgccggtgctgtgccaggcccatggcctgaccccggcgcaggtggtggccatcgccagccacgatggcggcaagcaggcgctggagacggtgcagcggctgttgccggtgctgtgccaggcccatggcctgaccccggaccaagtggtggccatcgccagcaatggcggcggcaagcaggcgctggagacggtgcagcggctgttgccggtgctgtgccaggcccatggcctgacccaggagcaggtggtggccatcgccagcaataacggcggcaagcaggcgctggagacggtgcagcggctgttgccggtgctgtgccaggcccatggcctgaccccggaccaggtggtggccatcgccagcaatggcggcggcaagcaggcgctggagacggtgcagcggctgttgccggtgctgtgccaggcccatggtctgaccccggcgcaggtggtggccatcgccagcaatattggcggcaagcaggcgctggagacggtgcagcggctgttgccggtgctgtgccaggaccatggcctgaccctggcgcaggtggtggccatcgccagcaatattggcggcaagcaggcgctggagacggtgcagcggctgttgccggtgctgtgccaggcacatggcctgacccaggaccaggtggtggccatcgccagcaatattggcggcaagcaggcgctggagacggtgcagcggctgttgccggtgctgtgccaggaccatggcctgaccccggaccaggtcgtggccatcgccagcaatattggcggcaagcaggcgctggagacggtgcagcggctgttgccggtgctgtgccaggaccatggcctgaccctggaccaggtggtggccatcgccagcaatggcggcaagcaggcgctggagacggtgcagcggctgttgccggtgctgtgccaggaccatggactgaccccggaccaggtcgtggccatcgccagcaatagtggcggcaagcaggcgctggagacggtgcagcggctgttgccggtgctgtgccaggaccatggcctgaccccgaaccaggtggtggccatcgccagcaatggcggcaagcaggcgctggagagcattgttgcccagttatctcgccctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgccgactacgcgcaagtggttcgcgtgctggagtttttccagtgccactcccacccagcgtacgcatttgatgaggccatgacgcagttcgggatgagcaggaacgggttggtacagctctttcgcagagtgggcgtcaccgaactcgaagcccgcggtggaacgctccccccagcctcgcagcgttgggaccgtatcctccaggcatcagggatgaaaagggccaaaccgtcccctacttcagctcaaacaccggatcaggcgtctttgcatgcattcgccgattcgctggagcgtgaccttgatgcgcccagcccaatgcacgagggagatcagacgcgggcaagcagccgtaaacggtcccgatcggatcgtgctgtcaccggcccctccgcacagcaggctgtcgaggtgcgcgttcccgaacagcgcgatgcgctgcatttgcccctcagctggagggtaaaacgcccgcgtaccaggatctggggcggcctcccggatcctggtacgcccatggctgccgacctggcagcgtccagcaccgtgatgtgggaacaagatgcggaccccttcgcaggggcagcggatgatttcccggcattcaacgaagaggaactcgcatggttgatggagctattgcctcagtga

SEQ ID NO: 2 - protein, 1366 aa, Xanthomonas oryzae
MDPIRPRRPSPARELLPGPQPDRVQPTADRGVSAPAGSPLDGLPARRTVSRTRLPSPPAPSPAFSAGSFSDLLRPFDPSLLDTSLLDSMPAVGTPHTAAAPAEWDEAQSALRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPAQVVAIASNNGGKQALETVQRLLPVLCQAHGLTPAQVVAIASHDGGKQALETMQRLLPVLCQAHGLPPDQVVAIASNIGGKQALETVQRLLPVLCQAHGLTPDQVVAIASHGGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASNGGKQALETVQRLLPVLCQAHGLTPDQVVAIASHDGGKQALETVQRLLPVLCQTHGLTPAQVVAIASHDGGKQALETVQQLLPVLCQAHGLTPDQVVAIASNIGGKQALATVQRLLPVLCQAHGLTPDQVVAIASNGGGKQALETVQRLLPVLCQAHGLTPDQVVAIASNGGGKQALETVQRLLPVLCQAHGLTQVQVVAIASNIGGKQALETVQRLLPVLCQAHGLTPAQVVAIASHDGGKQALETVQRLLPVLCQAHGLTPDQVVAIASNGGGKQALETVQRLLPVLCQAHGLTQEQVVAIASNNGGKQALETVQRLLPVLCQAHGLTPDQVVAIASNGGGKQALETVQRLLPVLCQAHGLTPAQVVAIASNIGGKQALETVQRLLPVLCQDHGLTLAQVVAIASNIGGKQALETVQRLLPVLCQAHGLTQDQVVAIASNIGGKQALETVQRLLPVLCQDHGLTPDQVVAIASNIGGKQALETVQRLLPVLCQDHGLTLDQVVAIASNGGKQALETVQRLLPVLCQDHGLTPDQVVAIASNSGGKQALETVQRLLPVLCQDHGLTPNQVVAIASNGGKQALESIVAQLSRPDPALAALTNDHLVALACLGGRPAMDAVKKGLPHAPELIRRVNRRIGERTSHRVADYAQVVRVLEFFQCHSHPAYAFDEAMTQFGMSRNGLVQLFRRVGVTELEARGGTLPPASQRWDRILQASGMKRAKPSPTSAQTPDQASLHAFADSLERDLDAPSPMHEGDQTRASSRKRSRSDRAVTGPSAQQAVEVRVPEQRDALHLPLSWRVKRPRTRIWGGLPDPGTPMAADLAASSTVMWEQDADPFAGAADDFPAFNEEELAWLMELLPQZ

SEQ ID NO: 3 - DNA, 38 bp, Artificial Sequence
  Engineered DNA oligonucleotide target sequence for crystalization
tagatatgcatctccccctactgtacaccaccaaaagt

SEQ ID NO: 4 - DNA, 38 bp, Artificial Sequence
  Engineered DNA oligonucleotide target sequence for crystalization
ttttggtggtgtacagtagggggagatgcatatctaac

SEQ ID NO: 5 - DNA, 80 bp, Artificial Sequence
  Engineered DNA oligonucleotide target sequence for crystalization
gttagatatgcatctccccctactgtacaccaccaaaagtcaatctatacgtagagggggatgacatgtggtggttttca

SEQ ID NO: 6 - DNA, 82 bp, Artificial Sequence
  Engineered DNA oligonucleotide target sequence for crystalization
gttagatatgcatctccccctactgtacaccaccaaaagtgcaatctatacgtagagggggatgacatgtggtggttttcac

SEQ ID NO: 7 - DNA, 64 bp, Artificial Sequence
  Engineered DNA oligonucleotide target sequence for crystalization
gatatgcatctccccctactgtacaccaccaactatacgtagagggggatgacatgtggtggtt

SEQ ID NO: 8 - DNA, 66 bp, Artificial Sequence
  Engineered DNA oligonucleotide target sequence for crystalization
gatatgcatctccccctactgtacaccaccaaactatacgtagagggggatgacatgtggtggttt

SEQ ID NO: 9 - DNA, 66 bp, Artificial Sequence
  Engineered DNA oligonucleotide target sequence for crystalization
agatatgcatctccccctactgtacaccaccaatctatacgtagagggggatgacatgtggtggtt

SEQ ID NO: 10 - DNA, 68 bp, Artificial Sequence
  Engineered DNA oligonucleotide target sequence for crystalization
agatatgcatctccccctactgtacaccaccaaatctatacgtagagggggatgacatgtggtggttt

SEQ ID NO: 11 - DNA, 70 bp, Artificial Sequence
  Engineered DNA oligonucleotide target sequence for crystalization
agatatgcatctccccctactgtacaccaccaaaatctatacgtagagggggatgacatgtggtggtttt

SEQ ID NO: 12 - DNA, 72 bp, Artificial Sequence
  Engineered DNA oligonucleotide target sequence for crystalization
tagatatgcatctccccctactgtacaccaccaaaaatctatacgtagagggggatgacatgtggtggtttt

SEQ ID NO: 13 - DNA, 74 bp, Artificial Sequence
  Engineered DNA oligonucleotide target sequence for crystalization
tagatatgcatctccccctactgtacaccaccaaaagatctatacgtagagggggatgacatgtggtggttttc

SEQ ID NO: 14 - DNA, 76 bp, Artificial Sequence
  Engineered DNA oligonucleotide target sequence for crystalization
ttagatatgcatctccccctactgtacaccaccaaaagaatctatacgtagagggggatgacatgtggtggttttc

SEQ ID NO: 15 - DNA, 78 bp, Artificial Sequence
  Engineered DNA oligonucleotide target sequence for crystalization
ttagatatgcatctccccctactgtacaccaccaaaagtaatctatacgtagagggggatgacatgtggtggttttca

SEQ ID NO: 16 - DNA, 70 bp, Artificial Sequence
  Engineered DNA oligonucleotide target sequence for crystalization
tagatatgcatctccccctactgtacaccaccaaaaatctatacgtagagggggatgacatgtggtggtt

SEQ ID NO: 17 - DNA, 71 bp, Artificial Sequence
  Engineered DNA oligonucleotide target sequence for crystalization
tagatatgcatctccccctactgtacaccaccaaaaatctatacgtagagggggatgacatgtggtggttt

SEQ ID NO: 18 - DNA, 72 bp, Artificial Sequence
  Engineered DNA oligonucleotide target sequence for crystalization
tagatatgcatctccccctactgtacaccaccaaaaaatctatacgtagagggggatgacatgtggtggttt

SEQ ID NO: 19 - DNA, 72 bp, Artificial Sequence
  Engineered DNA oligonucleotide target sequence for crystalization
tagatatgcatctccccctactgtacaccaccaaaacaatctatacgtagagggggatgacatgtggtggtt

SEQ ID NO: 20 - DNA, 78 bp, Artificial Sequence
  Engineered DNA oligonucleotide target sequence for crystalization
gttagatatgcatctccccctactgtacaccaccaaaagtcaatctatacgtagagggggatgacatgtggtggtttt

SEQ ID NO: 21 - DNA, 79 bp, Artificial Sequence
  Engineered DNA oligonucleotide target sequence for crystalization
gttagatatgcatctccccctactgtacaccaccaaaagtcaatctatacgtagagggggatgacatgtggtggttttc

SEQ ID NO: 22 - DNA, 78 bp, Artificial Sequence
  Engineered DNA oligonucleotide target sequence for crystalization
ttagatatgcatctccccctactgtacaccaccaaaagtcaatctatacgtagagggggatgacatgtggtggttttc

SEQ ID NO: 23 - DNA, 76 bp, Artificial Sequence
  Engineered DNA oligonucleotide target sequence for crystalization
tagatatgcatctccccctactgtacaccaccaaaagtcaatctatacgtagagggggatgacatgtggtggtttt

SEQ ID NO: 24 - DNA, 77 bp, Artificial Sequence
  Engineered DNA oligonucleotide target sequence for crystalization
gttagatatgcatctccccctactgtacaccaccaaaagcaatctatacgtagagggggatgacatgtggtggtttt

SEQ ID NO: 25 - DNA, 78 bp, Artificial Sequence
  Engineered DNA oligonucleotide target sequence for crystalization
gttagatatgcatctccccctactgtacaccaccaaaagcaatctatacgtagagggggatgacatgtggtggttttc

SEQ ID NO: 26 - DNA, 77 bp, Artificial Sequence
  Engineered DNA oligonucleotide target sequence for crystalization
ttagatatgcatctccccctactgtacaccaccaaaagcaatctatacgtagagggggatgacatgtggtggttttc

SEQ ID NO: 27 - DNA, 75 bp, Artificial Sequence
  Engineered DNA oligonucleotide target sequence for crystalization
tagatatgcatctccccctactgtacaccaccaaaagcaatctatacgtagagggggatgacatgtggtggtttt

SEQ ID NO: 28 - DNA, 68 bp, Artificial Sequence
  Engineered DNA oligonucleotide target sequence for crystalization
gatatgcatctccccctactgtacaccaccaaaaatctatacgtagagggggatgacatgtggtggtt

SEQ ID NO: 29 - DNA, 69 bp, Artificial Sequence
  Engineered DNA oligonucleotide target sequence for crystalization
agatatgcatctccccctactgtacaccaccaaaaatctatacgtagagggggatgacatgtggtggtt

SEQ ID NO: 30 - DNA, 70 bp, Artificial Sequence
  Engineered DNA oligonucleotide target sequence for crystalization
agatatgcatctccccctactgtacaccaccaaaaatctatacgtagagggggatgacatgtggtggttt

SEQ ID NO: 31 - DNA, 74 bp, Artificial Sequence
  Engineered DNA oligonucleotide target sequence for crystalization
tagatatgcatctccccctactgtacaccaccaaaataatctatacgtagagggggatgacatgtggtggtttt

SEQ ID NO: 32 - DNA, 78 bp, Artificial Sequence
  Engineered DNA oligonucleotide target sequence for crystalization
tagatatgcatctccccctactgtacaccaccaaaaggtccaatctatacgtagagggggatgacatgtggtggtttt

SEQ ID NO: 33 - DNA, 74 bp, Artificial Sequence
  Engineered DNA oligonucleotide target sequence for crystalization
agatatgcatctccccctactgtacaccaccaaagttcaatctatacgtagagggggatgacatgtggtggttt

SEQ ID NO: 34 - DNA, 76 bp, Artificial Sequence
  Engineered DNA oligonucleotide target sequence for crystalization
agatatgcatctccccctactgtacaccaccaaaagttcaatctatacgtagagggggatgacatgtggtggtttt

SEQ ID NO: 35 - DNA, 78 bp, Artificial Sequence
  Engineered DNA oligonucleotide target sequence for crystalization
tagatatgcatctccccctactgtacaccaccaaaagtgcacatctatacgtagagggggatgacatgtggtggtttt

SEQ ID NO: 36 - DNA, 78 bp, Artificial Sequence
  Engineered DNA oligonucleotide target sequence for crystalization
tagatatgcatctccccctactgtacaccaccaaaaggtcaatctatacgtagagggggatgacatgtggtggttttc

SEQ ID NO: 37 - DNA, 29 bp, Xanthomonas oryzae
gcgtcggcaaacaggcgtccggcgcacgc

SEQ ID NO: 38 - DNA, 29 bp, Xanthomonas oryzae
gcgtgcgccggacgcctgtttgccgacgc

SEQ ID NO: 39 - DNA, 46 bp, Xanthomonas oryzae
cgtgagcaaggcctccagggctcgagcgccggaaaactgtttgccg

SEQ ID NO: 40 - DNA, 46 bp, Xanthomonas oryzae
cgtgagcaaggcctccagggctcgagcgccggataactgtttgccg

SEQ ID NO: 41 - DNA, 46 bp, Xanthomonas oryzae
cgtgagcaaggcctccagggctcgagcgccggaaatctgtttgccg

SEQ ID NO: 42 - DNA, 46 bp, Xanthomonas oryzae
cgtgagcaaggcctccagggctcgagcgccggacatctgtttgccg

SEQ ID NO: 43 - DNA, 46 bp, Xanthomonas oryzae
cgtgagcaaggcctccagggctcgagcgccggagacctgtttgccg

SEQ ID NO: 44 - DNA, 46 bp, Xanthomonas oryzae
cgtgagcaaggcctccagggctcgagcgccggaagactgtttgccg

SEQ ID NO: 45 - DNA, 46 bp, Xanthomonas oryzae
cgtgagcaaggcctccagggctcgagcgccggatggctgtttgccg

SEQ ID NO: 46 - DNA, 46 bp, Xanthomonas oryzae
cgtgagcaaggcctccagggctcgagcgccggatgtctgtttgccg

SEQ ID NO: 47 - DNA, 46 bp, Xanthomonas oryzae
cgtgagcaaggcctccagggctcgagcgccggaatactgtttgccg

SEQ ID NO: 48 - DNA, 46 bp, Xanthomonas oryzae
cgtgagcaaggcctccagggctcgagcgccggaatgctgtttgccg

SEQ ID NO: 49 - DNA, 46 bp, Xanthomonas oryzae
cgtgagcaaggcctccagggctcgagcgccggattgctgtttgccg

SEQ ID NO: 50 - DNA, 46 bp, Xanthomonas oryzae
cgtgagcaaggcctccagggctcgagcgccggaattctgtttgccg

SEQ ID NO: 51 - DNA, 46 bp, Xanthomonas oryzae
cgtgagcaaggcctccagggctcgagcgccggatttctgtttgccg

SEQ ID NO: 52 - DNA, 46 bp, Xanthomonas oryzae
cgtgagcaaggcctccagggctcgagcgccggaatcctgtttgccg

SEQ ID NO: 53 - DNA, 46 bp, Xanthomonas oryzae
cgtgagcaaggcctccagggctcgagcgccggattcctgtttgccg

SEQ ID NO: 54 - DNA, 46 bp, Xanthomonas oryzae
cgtgagcaaggcctccagggctcgagcgccggaacactgtttgccg

SEQ ID NO: 55 - DNA, 46 bp, Xanthomonas oryzae
cgtgagcaaggcctccagggctcgagcgccggatcgctgtttgccg

SEQ ID NO: 56 - DNA, 46 bp, Xanthomonas oryzae
cgtgagcaaggcctccagggctcgagcgccggatccctgtttgccg

SEQ ID NO: 57 - DNA, 29 bp, Xanthomonas oryzae
cgcgtgcatctccccctactgtacaccac

SEQ ID NO: 58 - DNA, 29 bp, Xanthomonas oryzae
cgcggtggtgtacagtagggggagatgca

SEQ ID NO: 59 - DNA, 29 bp, Xanthomonas oryzae
cgcgagcatctccccctactgtacaccac

SEQ ID NO: 60 - DNA, 29 bp, Xanthomonas oryzae
cgcggtggtgtacagtagggggagatgct

SEQ ID NO: 61 - DNA, 29 bp, Xanthomonas oryzae
cgcgcgcatctccccctactgtacaccac

SEQ ID NO: 62 - DNA, 29 bp, Xanthomonas oryzae
cgcggtggtgtacagtagggggagatgcg

SEQ ID NO: 63 - DNA, 29 bp, Xanthomonas oryzae
cgcgggcatctccccctactgtacaccac

SEQ ID NO: 64 - DNA, 29 bp, Xanthomonas oryzae
cgcggtggtgtacagtagggggagatgcc

SEQ ID NO: 65 - DNA, 44 bp, Ralstonia solanacearum
ttgcatgtaaataggaggtgcaccatgagaataggcaaatcaag

SEQ ID NO: 66 - DNA, 29 bp, Ralstonia solanacearum
gagactcgtctcggcacgcgtgagcttcc

SEQ ID NO: 67 - DNA, 41 bp, Ralstonia solanacearum
cgggtagcagcgcttgcagcgccaggtcacccgaccactgc

SEQ ID NO: 68 - DNA, 41 bp, Ralstonia solanacearum
cgggtagcagcgcttgcagcgccaggtcacccgatgtctgc

SEQ ID NO: 69 - DNA, 41 bp, Ralstonia solanacearum
cgggtagcagcgcttgcagcgccaggtcacccgattgctgc

SEQ ID NO: 70 - DNA, 41 bp, Ralstonia solanacearum
cgggtagcagcgcttgcagcgccaggtcacccgatggctgc

SEQ ID NO: 71 - DNA, 41 bp, Ralstonia solanacearum
cgggtagcagcgcttgcagcgccaggtcacccgaattctgc

SEQ ID NO: 72 - DNA, 16 bp, Artificial Sequence
  Engineered DNA substrate for Electrophoretic mobility shift assay
aacgttaatggaagct

SEQ ID NO: 73 - DNA, 16 bp, Artificial Sequence
  Engineered DNA substrate for Electrophoretic mobility shift assay
cacgttaatggaagct

SEQ ID NO: 74 - DNA, 16 bp, Artificial Sequence
  Engineered DNA substrate for Electrophoretic mobility shift assay
gacgttaatggaagct

SEQ ID NO: 75 - DNA, 16 bp, Artificial Sequence
  Engineered DNA substrate for Electrophoretic mobility shift assay
tacgttaatggaagct

SEQ ID NO: 76 - DNA, 16 bp, Artificial Sequence
  Engineered DNA substrate for Electrophoretic mobility shift assay
tcgacgctcaggcaac

SEQ ID NO: 77 - protein, 1366 aa, Xanthomonas oryzae
MDPIRPRRPSPARELLPGPQPDRVQPTADRGVSAPAGSPLDGLPARRTVSRTRLPSPPAPSPAFSAGSFSDLLRPFDPSLLDTSLLDSMPAVGTPHTAAAPAEWDEAQSALRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVTYQHIITALPEATHEDIVGVGKQPSGARALEALLTDAGELRGPPLQLDTGQLVKIAKRGGVTAMEAVHASRNALTGAPLNLTPAQVVAIASNNGGKQALETVQRLLPVLCQAHGLTPAQVVAIASHDGGKQALETMQRLLPVLCQAHGLPPDQVVAIASNIGGKQALETVQRLLPVLCQAHGLTPDQVVAIASHGGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASNGGKQALETVQRLLPVLCQAHGLTPDQVVAIASHDGGKQALETVQRLLPVLCQTHGLTPAQVVAIASHDGGKQALETVQQLLPVLCQAHGLTPDQVVAIASNIGGKQALATVQRLLPVLCQAHGLTPDQVVAIASNGGGKQALETVQRLLPVLCQAHGLTPDQVVAIASNGGGKQALETVQRLLPVLCQAHGLTQVQVVAIASNIGGKQALETVQRLLPVLCQAHGLTPAQVVAIASHDGGKQALETVQRLLPVLCQAHGLTPDQVVAIASNGGGKQALETVQRLLPVLCQAHGLTQEQVVAIASNNGGKQALETVQRLLPVLCQAHGLTPDQVVAIASNGGGKQALETVQRLLPVLCQAHGLTPAQVVAIASNIGGKQALETVQRLLPVLCQDHGLTLAQVVAIASNIGGKQALETVQRLLPVLCQAHGLTQDQVVAIASNIGGKQALETVQRLLPVLCQDHGLTPDQVVAIASNIGGKQALETVQRLLPVLCQDHGLTLDQVVAIASNGGKQALETVQRLLPVLCQDHGLTPDQVVAIASNSGGKQALETVQRLLPVLCQDHGLTPNQVVAIASNGGKQALESIVAQLSRPDPALAALTNDHLVALACLGGRPAMDAVKKGLPHAPELIRRVNRRIGERTSHRVADYAQVVRVLEFFQCHSHPAYAFDEAMTQFGMSRNGLVQLFRRVGVTELEARGGTLPPASQRWDRILQASGMKRAKPSPTSAQTPDQASLHAFADSLERDLDAPSPMHEGDQTRASSRKRSRSDRAVTGPSAQQAVEVRVPEQRDALHLPLSWRVKRPRTRIWGGLPDPGTPMAADLAASSTVMWEQDADPFAGAADDFPAFNEEELAWLMELLPQZ

SEQ ID NO: 78 - DNA, 2091 bp, Xanthomonas oryzae
atggtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagcaacattggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcctaa

SEQ ID NO: 79 - protein, 696 aa, Xanthomonas oryzae
MVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNIGGKQALETVQRLLPVLCQDHGLTPDQVVAIASHDGGKQALETVQRLLPVLCQDHGLTPDQVVAIASNNGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASHDGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVA

SEQ ID NO: 80 - DNA, 4296 bp, Artificial Sequence
  Chimeric nucleotide sequence encoding PthXo1 from Xanthomonas oryzae in which the N-terminal region is replaced by the corresponding region from Ralstonia TAL effector-like protein
atgagaataggcaaatcaagcggttggttgaacgagtccgtgtctcttgaatatgaacacgtgtctccaccgacacggcctcgagacacccgtcgccggcctcgcgccgccggcgacggcgggctcgcgcatctgcatcgccggctcgcggtcggctacgcggaggacacgccgagaaccgaggctcggtctccggcgccgcgccgcccgctccctgtggcacctgcatccgcaccgcctgcaccatccctcgttccggaaccccctatgccggtcagccttcctgccgtatcgagcccgcgcttctctgccggcagctcggcagccatcaccgatccttttccgagccttccgcccacgcccgtgctgtatgcgatggctcgcgaactggaggcgctgtccgacgctacctggcagccagccgtaccgttgcccgccgagccgcccactgatgcgcggcgcggcaacacggtatttgacgaagcgtctgcatcatcgccggtgatcgcctcggcctgccctcaagcgtttgccagcccaccgcgagcaccgcgctcggcgcgagcccgcagggctcggacaggcggtgatgcttggccggccccgacttttcttagccgcccctcgtcatcccgcatcggccgtgacgtgttcgggaaactggtcgcactcggctattcccgtgagcagatccggaagctcaagcaggagagcctgagcgaaattgcgaagtatcacaccaccttgacaggacaagggttcacgcacgccgacatctgccggatcagccgcagacggcagtcgctccgggtggtcgccaggaactacccggagttggctgcggcgctccctgagctgaccagggcccacatcgtggacatcgctcggcagcgatcgggcgacttggcgctgcaagcgctgctacccgtggcgaccgcactgacagcggcccccctgagattgagcgcctcgcaaatcgcgaccgttgcgcagtacggcgagcggccggccatccaggccctttatcggctgcgccggaagctcacgcgtgcccccctgaacctgaccccggcacaggtggtggccatcgccagcaataacggtggcaagcaggcgctggagacggtgcagcggctgttgccggtgctgtgccaggcccatggcctgaccccggcgcaggtggtggccatcgccagccacgatggcggcaagcaggcactggagacgatgcagcggctgttgccggtgctgtgccaggcccatggcctgcccccggaccaggtggtggccatcgccagcaatattggcggcaagcaggcgctggagacggtgcagcggctgttgccggtgctctgccaggcccatggcctgaccccggaccaggtggtggccatcgccagccatggcggcggcaagcaggcgctggagacggtgcagcggctgttgccggtgctctgccaggcccatggcctgaccccggaccaggtggtggccatcgccagccacgatggcggcaagcaggcgctggagacggtgcagcggctgttgccggtgctgtgccaggcccatggcctgaccccggaccaggtggtggccatcgccagcaatggcggcggcaagcaggcgctggagacggtgcagcggctgttgccggtgctgtgccaggcccatggtctgaccccggaccaggtggtggccatcgccagcaatggcggcaagcaggcgctggagacggtgcagcggctgttgccggtgctgtgccaggcccatggcctgaccccggaccaggtggtggccatcgccagccacgatggcggcaagcaggcgctggagacggtgcagcggctgttgccggtgctgtgccagacccatggtctgaccccggcgcaggtggtggccatcgccagccacgatggcggcaagcaggcgctggagacggtgcagcagctgttgccggtgctgtgccaggcccatggcctgaccccggaccaggtggtggccatcgccagcaatattggcggcaagcaggcgctagcgacggtgcagcggctgttgccggtgctgtgccaagcccatggcctgaccccggaccaggtggtggccatcgccagcaatggcggcggcaagcaggcgctggagacggtgcagcggctgttgccggtgctgtgccaggcccatggcctgaccccggaccaggtggtggccatcgccagcaatggcggcggcaagcaggcgctggagacggtgcagcggctgttgccggtgctgtgccaggcccatggtctgacccaggtgcaggtggtggccatcgccagcaatattggcggcaagcaggcgctggagacggtgcagcggctgttgccggtgctgtgccaggcccatggcctgaccccggcgcaggtggtggccatcgccagccacgatggcggcaagcaggcgctggagacggtgcagcggctgttgccggtgctgtgccaggcccatggcctgaccccggaccaagtggtggccatcgccagcaatggcggcggcaagcaggcgctggagacggtgcagcggctgttgccggtgctgtgccaggcccatggcctgacccaggagcaggtggtggccatcgccagcaataacggcggcaagcaggcgctggagacggtgcagcggctgttgccggtgctgtgccaggcccatggcctgaccccggaccaggtggtggccatcgccagcaatggcggcggcaagcaggcgctggagacggtgcagcggctgttgccggtgctgtgccaggcccatggtctgaccccggcgcaggtggtggccatcgccagcaatattggcggcaagcaggcgctggagacggtgcagcggctgttgccggtgctgtgccaggaccatggcctgaccctggcgcaggtggtggccatcgccagcaatattggcggcaagcaggcgctggagacggtgcagcggctgttgccggtgctgtgccaggcacatggcctgacccaggaccaggtggtggccatcgccagcaatattggcggcaagcaggcgctggagacggtgcagcggctgttgccggtgctgtgccaggaccatggcctgaccccggaccaggtcgtggccatcgccagcaatattggcggcaagcaggcgctggagacggtgcagcggctgttgccggtgctgtgccaggaccatggcctgaccctggaccaggtggtggccatcgccagcaatggcggcaagcaggcgctggagacggtgcagcggctgttgccggtgctgtgccaggaccatggactgaccccggaccaggtcgtggccatcgccagcaatagtggcggcaagcaggcgctggagacggtgcagcggctgttgccggtgctgtgccaggaccatggcctgaccccgaaccaggtggtggccatcgccagcaatggcggcaagcaggcgctggagagcattgttgcccagttatctcgccctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgccgactacgcgcaagtggttcgcgtgctggagtttttccagtgccactcccacccagcgtacgcatttgatgaggccatgacgcagttcgggatgagcaggaacgggttggtacagctctttcgcagagtgggcgtcaccgaactcgaagcccgcggtggaacgctccccccagcctcgcagcgttgggaccgtatcctccaggcatcagggatgaaaagggccaaaccgtcccctacttcagctcaaacaccggatcaggcgtctttgcatgcattcgccgattcgctggagcgtgaccttgatgcgcccagcccaatgcacgagggagatcagacgcgggcaagcagccgtaaacggtcccgatcggatcgtgctgtcaccggcccctccgcacagcaggctgtcgaggtgcgcgttcccgaacagcgcgatgcgctgcatttgcccctcagctggagggtaaaacgcccgcgtaccaggatctggggcggcctcccggatcctggtacgcccatggctgccgacctggcagcgtccagcaccgtgatgtgggaacaagatgcggaccccttcgcaggggcagcggatgatttcccggcattcaacgaagaggaactcgcatggttgatggagctattgcctcagtga

SEQ ID NO: 81 - protein, 1432 aa, Artificial Sequence
  Chimeric amino acid sequence of PthXo1 from Xanthomonas oryzae in which the N-terminal region is replaced by the corresponding region from Ralstonia TAL effector-like protein
MRIGKSSGWLNESVSLEYEHVSPPTRPRDTRRRPRAAGDGGLAHLHRRLAVGYAEDTPRTEARSPAPRRPLPVAPASAPPAPSLVPEPPMPVSLPAVSSPRFSAGSSAAITDPFPSLPPTPVLYAMARELEALSDATWQPAVPLPAEPPTDARRGNTVFDEASASSPVIASACPQAFASPPRAPRSARARRARTGGDAWPAPTFLSRPSSSRIGRDVFGKLVALGYSREQIRKLKQESLSEIAKYHTTLTGQGFTHADICRISRRRQSLRVVARNYPELAAALPELTRAHIVDIARQRSGDLALQALLPVATALTAAPLRLSASQIATVAQYGERPAIQALYRLRRKLTRAPLNLTPAQVVAIASNNGGKQALETVQRLLPVLCQAHGLTPAQVVAIASHDGGKQALETMQRLLPVLCQAHGLPPDQVVAIASNIGGKQALETVQRLLPVLCQAHGLTPDQVVAIASHGGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASNGGKQALETVQRLLPVLCQAHGLTPDQVVAIASHDGGKQALETVQRLLPVLCQTHGLTPAQVVAIASHDGGKQALETVQQLLPVLCQAHGLTPDQVVAIASNIGGKQALATVQRLLPVLCQAHGLTPDQVVAIASNGGGKQALETVQRLLPVLCQAHGLTPDQVVAIASNGGGKQALETVQRLLPVLCQAHGLTQVQVVAIASNIGGKQALETVQRLLPVLCQAHGLTPAQVVAIASHDGGKQALETVQRLLPVLCQAHGLTPDQVVAIASNGGGKQALETVQRLLPVLCQAHGLTQEQVVAIASNNGGKQALETVQRLLPVLCQAHGLTPDQVVAIASNGGGKQALETVQRLLPVLCQAHGLTPAQVVAIASNIGGKQALETVQRLLPVLCQDHGLTLAQVVAIASNIGGKQALETVQRLLPVLCQAHGLTQDQVVAIASNIGGKQALETVQRLLPVLCQDHGLTPDQVVAIASNIGGKQALETVQRLLPVLCQDHGLTLDQVVAIASNGGKQALETVQRLLPVLCQDHGLTPDQVVAIASNSGGKQALETVQRLLPVLCQDHGLTPNQVVAIASNGGKQALESIVAQLSRPDPALAALTNDHLVALACLGGRPAMDAVKKGLPHAPELIRRVNRRIGERTSHRVADYAQVVRVLEFFQCHSHPAYAFDEAMTQFGMSRNGLVQLFRRVGVTELEARGGTLPPASQRWDRILQASGMKRAKPSPTSAQTPDQASLHAFADSLERDLDAPSPMHEGDQTRASSRKRSRSDRAVTGPSAQQAVEVRVPEQRDALHLPLSWRVKRPRTRIWGGLPDPGTPMAADLAASSTVMWEQDADPFAGAADDFPAFNEEELAWLMELLPQZ

SEQ ID NO: 82 - DNA, 4098 bp, Xanthomonas oryzae
atggatcccattcgtccgcgcaggccaagtcctgcccgcgagcttctgcccggaccccaaccggatagggttcagccgactgcagatcgtggggtgtctgcgcctgctggcagccctctggatggcttgcccgctcggcggacggtgtcccggacccggctgccatctccccctgcgccctcacctgcgttctcggcgggcagcttcagcgatctgctccgtccgttcgatccgtcgcttcttgatacatcgcttcttgattcgatgcctgccgtcggcacgccgcatacagcggctgccccagcagagtgggatgaggcgcaatcggctctgcgtgcagccgatgacccgccacccaccgtgcgtgtcgctgtcactgccgcgcggccgccgcgcgccaagccggccccgcgacggcgtgctgcgcaaccctccgacgcttcgccggccgcgcaggtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagccatccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggcacaggtggtggccatcgccagcaataacggtggcaagcaggcgctggagacggtgcagcggctgttgccggtgctgtgccaggcccatggcctgaccccggcgcaggtggtggccatcgccagccacgatggcggcaagcaggcactggagacgatgcagcggctgttgccggtgctgtgccaggcccatggcctgcccccggaccaggtggtggccatcgccagcaatattggcggcaagcaggcgctggagacggtgcagcggctgttgccggtgctctgccaggcccatggcctgaccccggaccaggtggtggccatcgccagccatggcggcggcaagcaggcgctggagacggtgcagcggctgttgccggtgctctgccaggcccatggcctgaccccggaccaggtggtggccatcgccagccacgatggcggcaagcaggcgctggagacggtgcagcggctgttgccggtgctgtgccaggcccatggcctgaccccggaccaggtggtggccatcgccagcaatggcggcggcaagcaggcgctggagacggtgcagcggctgttgccggtgctgtgccaggcccatggtctgaccccggaccaggtggtggccatcgccagcaatggcggcaagcaggcgctggagacggtgcagcggctgttgccggtgctgtgccaggcccatggcctgaccccggaccaggtggtggccatcgccagccacgatggcggcaagcaggcgctggagacggtgcagcggctgttgccggtgctgtgccagacccatggtctgaccccggcgcaggtggtggccatcgccagccacgatggcggcaagcaggcgctggagacggtgcagcagctgttgccggtgctgtgccaggcccatggcctgaccccggaccaggtggtggccatcgccagcaatattggcggcaagcaggcgctagcgacggtgcagcggctgttgccggtgctgtgccaagcccatggcctgaccccggaccaggtggtggccatcgccagcaatggcggcggcaagcaggcgctggagacggtgcagcggctgttgccggtgctgtgccaggcccatggcctgaccccggaccaggtggtggccatcgccagcaatggcggcggcaagcaggcgctggagacggtgcagcggctgttgccggtgctgtgccaggcccatggtctgacccaggtgcaggtggtggccatcgccagcaatattggcggcaagcaggcgctggagacggtgcagcggctgttgccggtgctgtgccaggcccatggcctgaccccggcgcaggtggtggccatcgccagccacgatggcggcaagcaggcgctggagacggtgcagcggctgttgccggtgctgtgccaggcccatggcctgaccccggaccaagtggtggccatcgccagcaatggcggcggcaagcaggcgctggagacggtgcagcggctgttgccggtgctgtgccaggcccatggcctgacccaggagcaggtggtggccatcgccagcaataacggcggcaagcaggcgctggagacggtgcagcggctgttgccggtgctgtgccaggcccatggcctgaccccggaccaggtggtggccatcgccagcaatggcggcggcaagcaggcgctggagacggtgcagcggctgttgccggtgctgtgccaggcccatggtctgaccccggcgcaggtggtggccatcgccagcaatattggcggcaagcaggcgctggagacggtgcagcggctgttgccggtgctgtgccaggaccatggcctgaccctggcgcaggtggtggccatcgccagcaatattggcggcaagcaggcgctggagacggtgcagcggctgttgccggtgctgtgccaggcacatggcctgacccaggaccaggtggtggccatcgccagcaatattggcggcaagcaggcgctggagacggtgcagcggctgttgccggtgctgtgccaggaccatggcctgaccccggaccaggtcgtggccatcgccagcaatattggcggcaagcaggcgctggagacggtgcagcggctgttgccggtgctgtgccaggaccatggcctgaccctggaccaggtggtggccatcgccagcaatggcggcaagcaggcgctggagacggtgcagcggctgttgccggtgctgtgccaggaccatggactgaccccggaccaggtcgtggccatcgccagcaatagtggcggcaagcaggcgctggagacggtgcagcggctgttgccggtgctgtgccaggaccatggcctgaccccgaaccaggtggtggccatcgccagcaatggcggcaagcaggcgctggagagcattgttgcccagttatctcgccctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgccgactacgcgcaagtggttcgcgtgctggagtttttccagtgccactcccacccagcgtacgcatttgatgaggccatgacgcagttcgggatgagcaggaacgggttggtacagctctttcgcagagtgggcgtcaccgaactcgaagcccgcggtggaacgctccccccagcctcgcagcgttgggaccgtatcctccaggcatcagggatgaaaagggccaaaccgtcccctacttcagctcaaacaccggatcaggcgtctttgcatgcattcgccgattcgctggagcgtgaccttgatgcgcccagcccaatgcacgagggagatcagacgcgggcaagcagccgtaaacggtcccgatcggatcgtgctgtcaccggcccctccgcacagcaggctgtcgaggtgcgcgttcccgaacagcgcgatgcgctgcatttgcccctcagctggagggtaaaacgcccgcgtaccaggatctggggcggcctcccggatcctggtacgcccatggctgccgacctggcagcgtccagcaccgtgatgtgggaacaagatgcggaccccttcgcaggggcagcggatgatttcccggcattcaacgaagaggaactcgcatggttgatggagctattgcctcagtga

(19) United States
(12) Patent Application Publication
(10) Pub. No.: US 2014/0186957 A1
(43) Pub. Date: Jul. 3, 2014

(54) ENGINEERED TAL EFFECTOR PROTEINS WITH ENHANCED DNA TARGETING CAPACITY

(71) Applicants: IOWA STATE UNIVERSITY RESEARCH FOUNDATION, INC., Ames, IA (US); FRED HUTCHINSON CANCER RESEARCH CENTER, Seattle, WA (US)

(72) Inventors: Adam Joseph Bogdanove, Ithaca, NY (US); Barry Leonard Stoddard, Bellevue, WA (US); Philip H. Bradley, Seattle, WA (US); Erin Lynn Doyle, Ames, IA (US)

(73) Assignees: Fred Hutchinson Cancer Research Center, Seattle, WA (US); Iowa State University Research Foundation, Inc., Ames, IA (US)

(21) Appl. No.: 13/733,442

(22) Filed: Jan. 3, 2013

(51) Int. Cl. C12N 15/01; C12N 9/16

(52) U.S. Cl. CPC C12N 15/01; C12N 9/16
USPC 435/441; 435/455; 435/468; 435/471; 435/188; 435/325; 435/419; 435/243; 435/320.1; 536/23.2

(57) ABSTRACT

The present invention provides compositions and methods for DNA targeting using TAL effectors and TAL effector based proteins, including but not limited to targeted gene regulation and targeted cleavage of cellular chromatin in a region of interest and/or homologous recombination at a predetermined site in cells. Compositions include fusion polypeptides comprising a TAL effector or a TAL effector binding domain in combination with other domains, including but not limited to a cleavage domain. The TAL effector binding domain includes modifications that increase activity of the same and also remove the constraints that the DNA target sequence be preceded by a thymine.

### STATEMENT REGARDING FEDERALLY SPONSORED RESEARCH OR DEVELOPMENT

This invention was made with Government support under Grant Nos. RL1 CA833133, R01GM098861, and R01 GM088277 from the National Institutes of Health and Grant No. 0820831 from the National Science Foundation. The Government has certain rights in the invention.

### BACKGROUND OF THE INVENTION

1. Field of the Invention

This invention relates to methods for homologous recombination and gene targeting, and particularly to methods that include the use of transcription activator-like (TAL) effector sequences.

2. Background

TAL effectors belong to a large group of bacterial proteins that exist in various strains of Xanthomonas spp. and are translocated into host cells by a type III secretion system, so called type III effectors. Once in host cells, some TAL effectors have been found to transcriptionally activate their corresponding host target genes either for strain virulence (ability to cause disease) or avirulence (capacity to trigger host resistance responses) dependent on the host genetic context. Each effector contains the functional nuclear localization motifs and a potent transcription activation domain that are characteristic of eukaryotic transcription activators. Each effector also contains a central repetitive region consisting of varying numbers of repeat units of (typically) 34 amino acids, and the repeat region as DNA binding domain determines the biological specificity of each effector. The repeat is nearly identical except for the variable amino acids at positions 12 and 13, so called repeat variable di-residues (RVD), of each repeat. Recent studies have revealed the recognition of DNA sequences within the promoters of host target genes by the repeat regions of TAL effectors, and the recognition could be simplified in a code that one nucleotide of a target site is corresponding in a sequential order to the RVD of one repeat, with the tandem array of repeats corresponding to a specific, consecutive stretch of DNA. The majority of naturally occurring TAL effector proteins contain repeat units in a range of 13 to 29 repeats that presumably recognize DNA elements consisting of same number of nucleotides. The TAL effector domain that binds to a specific nucleotide sequence within the target DNA can comprise 10 or more DNA binding repeats, and preferably 15 or more DNA binding repeats. Each DNA binding repeat can include a repeat variable-diresidue (RVD) that determines recognition of a base pair in the target DNA sequence, wherein each DNA binding repeat is responsible for recognizing one base pair in the target DNA sequence, and wherein the RVD comprises one or more of: HD for recognizing C; NG for recognizing T; NI for recognizing A; NN for recognizing G or A; NH for recognizing G; NS for recognizing A or C or G or T; N* for recognizing C or T, where * represents a gap in the second position of the RVD; HG for recognizing T; H* for recognizing T, where * represents a gap in the second position of the RVD; IG for recognizing T; NK for recognizing G; HA for recognizing C; ND for recognizing C; HI for recognizing C; FIN for recognizing G; NA for recognizing G; SN for recognizing G or A; and YG for recognizing T. Each DNA binding repeat can comprise a RVD that determines recognition of a base pair in the target DNA sequence, wherein each DNA binding repeat is responsible for recognizing one base pair in the target DNA sequence, and wherein the RVD comprises one or more of: HA for recognizing C; ND for recognizing C; HI for recognizing C; HN for recognizing G; NA for recognizing G; SN for recognizing G or A; YG for recognizing T; and NK for recognizing G, and one or more of: HD for recognizing C; NG for recognizing T; NI for recognizing A; NN for recognizing G or A; NS for recognizing A or C or G or T; N* for recognizing C or T, wherein * represents a gap in the second position of the RVD; HG for recognizing T; H* for recognizing T, wherein * represents a gap in the second position of the RVD; and IG for recognizing T.

Compositions and methods for targeted cleavage of cellular chromatin in a region of interest and/or homologous recombination at a predetermined region of interest in cells using restriction endonucleases, TAL effectors and cleavage domain fusion proteins is disclosed in Voytas et al., U.S. Publication number 20120214228 filed Mar. 22, 2012, the disclosure of which is hereby incorporated in its entirety, specifically paragraphs 111 through 115, 143 through 166, and 193 through 263. Cells include cultured cells, cells in an organism and cells that have been removed from an organism for treatment in cases where the cells and/or their descendants will be returned to the organism after treatment. A region of interest in cellular chromatin can be, for example, a genomic sequence or portion thereof. Compositions include fusion polypeptides comprising a TAL effector binding domain and a cleavage domain. The cleavage domain can be from any endonuclease, preferably a Type II S restriction endonuclease.

It is an object of the present invention to provide novel mechanisms for the design of TAL effectors that can remove previous constraints requiring a 5′ thymine. Compositions, methods and altered DNA products such as chromatin, cells, nucleotides and the like are also included within the invention.

### BRIEF SUMMARY OF THE INVENTION

TAL effector proteins that occur in nature are uniformly initiated by a 5′ thymine, or in a single case, a 5′cytosine that precedes the nucleotide sequence specified by the TAL effector repeats (referred to as position 0). The current invention involves novel amino acid substitutions in the TAL effector protein N-terminal to the repeat region that remove this constraint. The invention broadens the targeting range of TAL effectors with custom repeat arrays to virtually any sequence of nucleotides in the DNA, enhancing the utility of TAL effectors as tools for custom gene regulation, genome engineering, and other DNA targeting applications. Thus the invention allows for the TAL effectors and TAL effector-based proteins to efficiently bind DNA sequences that begin with a thymine, cytosine, guanine, or adenine 5′ of the target sequence.

According to the invention, Applicants have discovered two degenerate N-terminal cryptic repeats which interact with the 5′ thymine in target DNA which may be modified to specify other bases such as cytosine, guanine, or adenine. The protein sequence immediately preceding the RVD-containing repeats was observed to have some similarity to the repeat consensus sequence and it was shown that this region comprises two repeats (termed 0 and −1), one or more of which, specify the thymine residue. Modification of these repeats can be made to specify cytosine, guanine, or adenine as desired for specific nucleotide sequence targeting. In the PthXo1 TAL effector, residues 221 to 239 and 256 to 273 were each found to interact indirectly with thymine base 5′ to the target DNA sequences. Trp 232 (−1^{st} repeat) forms a non polar van der Waals contact with the methyl carbon of the thymine base at position 0. This tryptophan was replaced with other amino acids and shown to alter specificity of the base target. For example, when a Trp 232 is replaced with a single amino acid, replacement with glutamine (Q) increased activity and altered specificity so that a cytosine was preferred; threonine (T) increased activity and eliminated all specificity; proline (P) increased activity and relaxed specificity to allow guanine or thymine at position 0; and asparagine (N) retained wild-type activity and eliminated all specificity at the 0 position. Other substitutions can be made based upon known RVDs such as NG, HD, NI, NN replacing either the W*, the QW, or the WS in the −1 repeat, or the R*, KR, or RG of the 0^{th} repeat.

Thus the invention comprises a method for targeting a DNA sequences in a cell, including targeting for the purpose of modifying the genetic material of a cell. The method may be applied to targeting of TAL effector nucleases (TALENS). The method may also be applied to other TAL effector based DNA targeting, including custom trascriptional activators, repressors, and DNA modifiers such as dioxygenases and methylases, among others. The method includes providing a primary cell containing a chromosomal target DNA sequence in which it is desired to have homologous recombination occur; providing a TAL effector endonuclease comprising an endonuclease domain that can cleave double stranded DNA, and a TAL effector domain comprising a plurality of TAL effector repeat sequences that, in combination, bind to a specific nucleotide sequence within the target DNA in the cell; and contacting the target DNA sequence with the TAL effector endonuclease in the cell such that the TAL effector endonuclease cleaves both strands of a nucleotide sequence within or adjacent to the target DNA sequence in the cell.

The method can further include providing a nucleic acid comprising a sequence homologous to at least a portion of the target DNA, such that homologous recombination occurs between the target DNA sequence and the nucleic acid. The target DNA sequence can be endogenous to the cell. The cell can be a plant cell or a mammalian cell. The contacting can include transfecting the cell with a vector comprising a TAL effector endonuclease coding sequence, and expressing the TAL effector endonuclease protein in the cell, mechanically injecting a TAL effector endonuclease protein into the cell, delivering a TAL effector endonuclease protein into the cell by means of the bacterial type III secretion system, or introducing a TAL effector endonuclease protein into the cell by electroporation. The TAL effector domain that binds to a specific nucleotide sequence within the target DNA can include 15 or more DNA binding repeats. The cell can be from an organism selected from the group consisting of a plant, an animal, a mammal, a human, a teleost fish, a fungus, a bacteria or a protozoa.

In another embodiment the invention includes a method for designing a sequence specific TAL effector endonuclease capable of cleaving DNA at a specific location. The method includes identifying a first unique endogenous chromosomal nucleotide sequence adjacent to a second nucleotide sequence at which it is desired to introduce a double-stranded cut; and designing a sequence specific TAL effector endonuclease comprising (a) a plurality of DNA binding repeat domains that, in combination, bind to the first unique endogenous chromosomal nucleotide sequence, wherein said design includes modifications in the 0^{th} and −1th repeat region 5′ of the RVD-containing repeat region to allow targeting of sequences with other than a thymine at position 0 or to increase activity and (b) an endonuclease that generates a double-stranded cut at the second nucleotide sequence.

In another embodiment, the invention includes a method for designing a PthXo1 TAL effector endonuclease that does not require a thymine in the 5′ position of the target sequence. The method includes modifying the amino acid sequence of the −1 and/or 0^{th} repeats of the TAL effector domain. The method further comprises the modification of other TAL effector proteins or TAL effector-like proteins to eliminate or modify the requirement for a 5′ thymine.

In another embodiment, the invention includes a composition comprising a TAL effector DNA binding domain and an endonuclease domain (TALEN) specific for a target DNA, wherein the repeats at the 0^{th} or −1st position are modified to eliminate the need for a thymine 5′ to the target DNA binding domain. The TALEN of the composition further includes DNA binding domains wherein a plurality of DNA binding repeats contain a RVD that determines recognition of a base pair in the target DNA, wherein each DNA binding repeat is responsible for recognizing one base pair in the target DNA. The invention further includes a TALEN wherein the endonuclease domain is from a type II restriction endonuclease, including the type II restriction endonuclease FokI. The invention further includes a TALEN wherein the TAL effector DNA binding domain is a Xanthomonas TAL effector, including but not limited to PthXo1, AvrXa7, PthXo3, and PthXo2. The invention also includes a TALEN wherein the TAL effector DNA binding domain is from a TAL effector-like protein of Ralstonia solanacearum. The invention also includes a TALEN wherein the TAL effector 0^{th} and −1^{st} repeat region is from a TAL effector-like protein of Ralstonia solanacearum.

According to the invention, the TALEN protein can be expressed in a cell, e.g., by delivering the fusion protein to the cell or by delivering a polynucleotide encoding the fusion protein to a cell, wherein the polynucleotide, if DNA, is transcribed, and an RNA molecule delivered to the cell or a transcript of a DNA molecule delivered to the cell is translated, to generate the fusion protein. Methods for polynucleotide and polypeptide delivery to cells are known in the art and are presented elsewhere in this disclosure.

Thus, the invention also includes a nucleic acid sequence which encodes a TAL effector fusion protein comprising an endonuclease domain and a TAL effector DNA binding domain specific for a target DNA, which has been designed to interact with and cleave a target sequence with any nucleotide at the 5′ position, as well as an expression construct, for example a vector, that includes such a nucleic acid sequence operably linked to a promoter sequence capable of directing expression in a cell, such as a mammalian, plant, other eukaryotic cell, or a prokaryotic cell.

Targeted mutations resulting from the aforementioned method include, but are not limited to, point mutations (i.e., conversion of a single base pair to a different base pair), substitutions (i.e., conversion of a plurality of base pairs to a different sequence of identical length), insertions or one or more base pairs, deletions of one or more base pairs and any combination of the aforementioned sequence alterations.

Methods for targeted recombination (for, e.g., alteration or replacement of a sequence in a chromosome or a region of interest in cellular chromatin) are also provided. For example, a mutant genomic sequence can be replaced by a wild-type sequence, e.g., for treatment of genetic disease or inherited disorders. In addition, a wild-type genomic sequence can be replaced by a mutant sequence, e.g., to prevent function of an oncogene product or a product of a gene involved in an inappropriate inflammatory response. Furthermore, one allele of a gene can be replaced by a different allele.

### BRIEF DESCRIPTION OF THE DRAWINGS

FIG. 1 shows domain organization of PthXo1 and structure of a single TAL effector repeat. (A) Bases shown in blue correspond to positions where physiological match between protein and target site differs from the optimal match specified by the TAL effector-DNA recognition code. Arrows indicate starting and ending positions of the construct used for crystallization. (B) The sequence and structure of a representative repeat (#14) is shown. (C) In the crystal structure, repeats 22 to 23.5 are poorly ordered, as are the C-terminal ends of the two N-terminal cryptic repeats (indicated in the cartoon by the fading color for those repeats). The ‘HD’ RVD residues that read out a cytosine are shown in red.

FIG. 2 shows the structure of the PthXo1 TAL effector DNA binding region in complex with its target site. The coloring of individual TAL repeats matches the schematic in FIG. 1. The left-hand representation shows the complex from a side view. The right-hand representation shows the complex from a top-down view.

FIG. 3 shows the topology and contacts between TAL effector repeats and DNA bases. (A) 8 distinct combinations of RVDs and DNA bases are observed in the structure. HD forms a steric and electrostatic contact with cytosine; HG and NG both form nonpolar interactions between the glycine α-carbon and the thymine methyl group. A “mismatch’ between NG and a cytosine results in a longer distance from the RVD to the base. NN associates with either guanine (repeat 16) or with adenine (which would interact with the same N7 nitrogen of the purine base). NI forms a desolvating interface with either adenine (repeat 3) or cytosine (repeat 19). The reduction in loop length by one residue in the ‘N*’ RVD (repeat 7) results in an increased distance to the base. (B) Two adjacent repeats form a tightly packed left-handed bundle of helices that position the second amino acid of each RVD in proximity to corresponding consecutive bases in an unperturbed B-form DNA duplex. The first residue of each RVD (position 12, either His or Asn) forms H-bonds to the backbone carbonyl oxygen of amino acid position 8 of the same repeat.

FIG. 4 shows N-terminal cryptic repeats and contacts with 5′ thymine. (A) 2Fo-Fc electron density maps contoured around thymine at position ‘0’ and tryptophan 232 in the ‘−1’ repeat. (B) Residues 221 to 239 and residues 256 to 273 each form a helix and an adjoining loop that resembles helix 1 and the RVD loop in the canonical repeats; the remaining residues in each region are poorly ordered. W232 forms a non polar van der Waals contact with the methyl carbon of the thymine base at position 0.

FIG. 5 shows the amino acid sequence of the PthXo1 protein construct used for crystallization trials. The additional N- and C-terminal regions of PthXo1 that are absent from the crystallization construct are shown in flanking grey text. Those additional residues in PthXo1 that differ from the corresponding residues in the crystallization construct are also shown in directly above their counterparts and are indicated with boxes.

Individual TAL repeats are aligned; the repeat variable diresidues (RVDs) are indicated (*) and all additional polymorphisms are underlined. The canonical repeats, numbered from 1, are preceded in the protein sequence and structure by two additional cryptic, degenerate repeats, numbered −1 and 0. The nucleotide sequence of the PthXo1 effector binding element in the rice Os8N3 gene promoter is indicated to the right. Arrows indicate bases that deviate from the base specified by the previously elucidated code for TAL effector-DNA recognition, and the code-specified base (in parentheses). Numbers in parentheses correspond to the full length PthXo1 protein.

FIG. 6 shows high-throughput computational modeling. Representative models after de novo model construction (panel a) and after several rounds of fragment-based refinement (panel b), shown in their unit cell placements as determined by molecular replacement searches.

FIG. 7 shows model-free validating electron density features. (A and B) Two strong peaks observed in an anomalous difference Fourier map, calculated using a low resolution SeMet dataset using phases corresponding to the initial computational model of the TAL-DNA complex, superimpose on the side chains of Met 272 (located in the ‘0’ repeat) and Met 344 (located in the 2nd repeat). While the SeMet dataset was too low in resolution to facilitate model building, the location of these peaks indicated that the phases from the computational model were highly accurate, and indicated the corresponding correct register of the TAL repeats. (C) Difference density in the N-terminal −1 repeat at the position of Trp 232 (which was initially modeled as an alanine) clearly indicated the presence and rotameric conformation of the indole side chain. (D) Difference density for individual TAL repeats (such as #8, shown here) after removal of the repeat model and simulated annealing refinement, was unambiguous and allowed independent rebuilding.

FIG. 8 shows Superposition of TAL repeats. Superposition of ‘HD’ containing repeats (top), ‘NG’ containing repeats (middle) and ‘NI’ containing repeats (bottom) indicate the strong structural similarity across the repeating fold. The divergence of one RVD loop in the middle panel corresponds to repeat number 11, in which an ‘NG’ RVD is associated with a ‘mis-matched’ cytosine residue.

FIG. 9 shows normalized GUS activity of PthXo1 variants containing amino acid substitutions for tryptophan 232 on UptPthXo1 targets with each possible nucleotide at position 0. The substitutions for the −1^{st} repeat tryptophan 232 are indicated in parentheses. UptA, UptC, UptG, and UptT denote the PthXo1 target preceded at position 0 by adenine, cytosine, guanine, or thymine, respectively. Each assay included a positive control of wild type PthXo1 (tryptophan 232 unchanged) on UptT. For comparison between assays, activity was normalized so that activity of this positive control was 1.0.

FIG. 10 shows activity of PthXo1 and all possible single amino acid substitution variants at position 232 on its target preceded by adenine, cytosine, guanine, or thymine. Agrobacterium mediated transient expression of the TAL effector was used to assay activity on a simultaneously Agrobacterium-delivered GUS reporter construct driven by the Bs3 promoter containing the target UPT box, in Nicotiana benthamiana. Substitutions at position 232 are indicated in parentheses. UptA, UptC, UptG, and UptT indicate activity on the target preceded by adenine, cytosine, guanine, or thymine, respectively. Activity is shown normalized to the activity of PthXo1 on UptT, set to 1.0. Error bars report the mean of four replicates±1 s.d. Data for each substitution are for one experiment. Experiments were repeated at least twice with similar results. Activity of the reporter constructs in the absence of the TAL effector (“None”) is shown at left.

FIG. 11 shows the effect of selected amino acid substitutions for tryptophan 232 on the relative binding affinities of a TAL effector for its target preceded by thymine (T), adenine (A), cytosine (C), or guanine (G). (A) Relative affinity of custom TAL effector TAL868 (WT) and variants with tryptophan 232 substituted by asparagine (W232N), proline (W232P), glutamine (W232Q), arginine (W232R), or threonine (W232T) for the TAL868 target preceded by T, A, C, or G, demonstrated by electrophoretic mobility shift assay. For each TAL effector target combination, protein concentration increases from left to right. Bands across the bottom represent unbound DNA. The next bands up represent DNA bound by the TAL effector. The uppermost bands represent higher order complexes. DNA bound at lower protein concentrations indicates higher affinity. (B) Fraction of DNA bound as a function of protein concentration, estimated by band densitometric analysis of the images in panel A, for TAL868 with the native tryptophan at position 232 (WT) and the W232P substituted version, on the target (substrate) preceded by T, A, C, or G.

FIG. 12 shows the activity of chimeric TAL effector −1st repeat variants. GUS activity for a chimeric TAL effector consisting of PthXo1 with its N-terminal region replaced by that of a Ralstonia TAL effector like protein, and variants with selected amino acid substitutions (W, tryptophan; P, proline: Q, glutamine; T, threonine; and N, asparagine) for the arginine (R) in the Ralstonia portion at the position that corresponds to tryptophan 232 in the PthXo1 N-terminal region. UptA, UptC, UptG, and UptT indicate the target UptPthXo1 preceded by an adenine, cytosine, guanine, or thymine, respectively. Activity was normalized to that of PthXo1 on UptT, set to 1.0. Activity of UptT in the absence of a TAL effector (None) was included as a negative control. Error bars report mean of four replicates±s.d.

FIG. 13 shows ClustalW multiple sequence alignment of N terminal sequences of Ralstonia TAL effector-like proteins (RTLs) and PthXo1 (SEQ ID NO:2). RSc1815, CAD15517.1 from Ralstonia solanacearum strain GMI10004. Hpx17, AB178011.1 from strain RS10855. RscCAQ18687, CAQ18687.1 from strain MolK2 (direct Genbank submission by Genoscope, C.E.A.). PthXo1, ACD58243.1 from Xanthomonas oryzae strain PX099A6. Tryptophan 232 and the arginines in the corresponding position in the Ralstonia proteins are in bold.

FIG. 14 shows secondary structure predictions for PthXo1 and Rsc1815 N terminal regions. Left: PthXo1, ACD58243.1 from Xanthomonas oryzae strain PX099A6.

Right: RSc1815 (GenBank protein CAD15517.1) from Ralstonia solanacearum strain GMI10004.

FIG. 15 (A-I) shows the nucleic acid and amino acid sequences of the various constructs used in this invention. The sequences are labeled 1-8. Sequence 1 (A-B) shows the DNA sequence of PthXo1 (SEQ ID NO:1) with the condon encoding W232 bolded and underlined. Sequence 2 (B-D) shows the DNA sequence of PthXo1 W232P mutant (SEQ ID NO:82), with the codon encoding the proline substituted for W232 bolded and underlined. Sequence 3 (D) shows the amino acid sequence of PthXo1 (SEQ ID NO:2) with W232 bolded and underlined. Sequence 4 (D-E) shows the amino acid sequence of PthXo1 W232P (SEQ ID NO:77) with the proline substitution for W232 bolded and underlined. Sequence 5 (E-F) shows the nucleotide sequence of dTALE868 (SEQ ID NO:78), with the condon encoding W232 bolded and underlined. Sequence 6 (F) shows the amino acid sequence of dTALE868 (SEQ ID NO:79), with W232 bolded and underlined. Sequence 7 (F-H) shows the nucleotide sequence of the chimeric PthXo1 construct substituted with a Ralstonia TAL effector-like protein N-terminal region (SEQ ID NO:80), which is indicated by italics. Sequence 8 (H—I) shows the amino acid sequence of the chimeric PthXo1 construct substituted with a Ralstonia TAL effector-like protein N-terminal region (SEQ ID NO:81), which is indicated by italics.

### DETAILED DESCRIPTION OF THE INVENTION

The patent or application file contains at least one drawing executed in color. Copies of this patent or patent application publication with color drawing(s) will be provided by the Office upon request and payment of the necessary fee.

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolfe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

### DEFINITIONS

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

A “TAL effector DNA binding protein” (or binding domain) or a “TAL effector DNA recognition sequence” is a protein encompassing a series of repeat variable-diresidues (RVDs) within a larger protein, that binds DNA in a sequence-specific manner. The RVD regions of TAL effectors are polymorphisms within TALs typically at positions 12 and 13 in repeating units of typically 34 amino acids that interact with specific nucleotides and together with a plurality of repeating unit intervals make up the specific TAL effector DNA binding domain.

TAL effector DNA binding protein domains (their RVDs) can be “engineered” to bind to a predetermined nucleotide sequence. Non-limiting examples of methods for engineering the same are design and selection. A designed TAL effector DNA binding protein is a protein not occurring in nature whose design/composition results principally from rational criteria. Rational criteria for design include application of substitution rules and computerized algorithms for processing information in a database storing information of existing RVD designs and binding data.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value there between or thereabove), preferably between about 100 and 1,000 nucleotides in length (or any integer there between), more preferably between about 200 and 500 nucleotides in length.

A “homologous, non-identical sequence” refers to a first sequence which shares a degree of sequence identity with a second sequence, but whose sequence is not identical to that of the second sequence. For example, a polynucleotide comprising the wild-type sequence of a mutant gene is homologous and non-identical to the sequence of the mutant gene. In certain embodiments, the degree of homology between the two sequences is sufficient to allow homologous recombination there between, utilizing normal cellular mechanisms. Two homologous non-identical sequences can be any length and their degree of non-homology can be as small as a single nucleotide (e.g., for correction of a genomic point mutation by targeted homologous recombination) or as large as 10 or more kilobases (e.g., for insertion of a gene at a predetermined ectopic site in a chromosome). Two polynucleotides comprising the homologous non-identical sequences need not be the same length. For example, an exogenous polynucleotide (i.e., donor polynucleotide) of between 20 and 10,000 nucleotides or nucleotide pairs can be used.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively.

Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present disclosure is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects sequence identity. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between homologous regions, followed by digestion with single-stranded-specific nuclease(s), and size determination of the digested fragments. Two nucleic acid, or two polypeptide sequences are substantially homologous to each other when the sequences exhibit at least about 70%-75%, preferably 80%-82%, more preferably 85%-90%, even more preferably 92%, still more preferably 95%, and most preferably 98% sequence identity over a defined length of the molecules, as determined using the methods above. As used herein, substantially homologous also refers to sequences showing complete identity to a specified DNA or polypeptide sequence. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press).

Conditions for hybridization are well-known to those of skill in the art. Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations.

With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. The selection of a particular set of hybridization conditions is selected following standard methods in the art (see, for example, Sambrook, et al., Molecular Cloning: A Laboratory Manual, Second Edition, (1989) Cold Spring Harbor, N.Y.).

“Recombination” refers to a process of exchange of genetic information between two polynucleotides. For the purposes of this disclosure, “homologous recombination (HR)” refers to the specialized form of such exchange that takes place, for example, during repair of double-strand breaks in cells. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “cleavage domain” comprises one or more polypeptide sequences which possesses catalytic activity for DNA cleavage. A cleavage domain can be contained in a single polypeptide chain or cleavage activity can result from the association of two (or more) polypeptides.

“Chromatin” is the nucleoprotein structure comprising the cellular genome. Cellular chromatin comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) extends between nucleosome cores. A molecule of histone H1 is generally associated with the linker DNA. For the purposes of the present disclosure, the term “chromatin” is meant to encompass all types of cellular nucleoprotein, both prokaryotic and eukaryotic. Cellular chromatin includes both chromosomal and episomal chromatin.

A “chromosome,” is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

An “accessible region” is a site in cellular chromatin in which a target site present in the nucleic acid can be bound by an exogenous molecule which recognizes the target site. Without wishing to be bound by any particular theory, it is believed that an accessible region is one that is not packaged into a nucleosomal structure. The distinct structure of an accessible region can often be detected by its sensitivity to chemical and enzymatic probes, for example, nucleases.

A “target site” or “target sequence” is a nucleic acid sequence that defines a portion of a nucleic acid to which a binding molecule will bind, provided sufficient conditions for binding exist. For example, the sequence 5′-GAATTC-3′ is a target site for the Eco RI restriction endonuclease.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylates, demethylases, acetylases, deacetylases, kinases, phosphatases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “fusion” molecule is a molecule in which two or more subunit molecules are linked, preferably covalently. The subunit molecules can be the same chemical type of molecule, or can be different chemical types of molecules. Examples of the first type of fusion molecule include, but are not limited to, fusion proteins (for example, a fusion between a TAL effector sequence DNA-binding domain and a cleavage domain) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid.

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” of gene expression refers to a change in the activity of a gene. Modulation of expression can include, but is not limited to, gene activation and gene repression.

“Eucaryotic” cells include, but are not limited to, fungal cells (such as yeast), plant cells, animal cells, mammalian cells and human cells.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

With respect to fusion polypeptides, the term “operatively linked” can refer to the fact that each of the components performs the same function in linkage to the other component as it would if it were not so linked. For example, with respect to a fusion polypeptide in which a TAL effector DNA-binding domain is fused to a cleavage domain, the TAL effector DNA-binding domain and the cleavage domain are in operative linkage if, in the fusion polypeptide, the TAL effector DNA-binding domain portion is able to bind its target site and/or its binding site, while the cleavage domain is able to cleave DNA in the vicinity of the target site.

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and PCT WO 98/44350.

### TAL Effector Mediated Modification

The invention comprises a method for modifying the genetic material of a cell using TAL effector mediated modification in which TAL effector protein N-terminal sequences are modified to remove the requirement of a thymine residue in target DNA sequences, to specify other specific residues, to remove all specificity and/or, in some circumstances to increase activity of the TAL effector.

The method includes providing a primary cell containing a chromosomal target DNA sequence in which it is desired to have homologous recombination occur; providing a TAL effector endonuclease comprising an endonuclease domain that can cleave double stranded DNA, and a TAL effector domain comprising a plurality of TAL effector repeat sequences that, in combination, bind to a specific nucleotide sequence within the target DNA in the cell; and contacting the target DNA sequence with the TAL effector endonuclease in the cell such that the TAL effector endonuclease cleaves both strands of a nucleotide sequence within or adjacent to the target DNA sequence in the cell. The method can further include providing a nucleic acid comprising a sequence homologous to at least a portion of the target DNA, such that homologous recombination occurs between the target DNA sequence and the nucleic acid. The target DNA sequence can be endogenous to the cell. The cell can be a plant cell or a mammalian cell. The contacting can include transfecting the cell with a vector comprising a TAL effector endonuclease coding sequence, and expressing the TAL effector endonuclease protein in the cell, mechanically injecting a TAL effector endonuclease protein into the cell, delivering a TAL effector endonuclease protein into the cell by means of the bacterial type III secretion system, or introducing a TAL effector endonuclease protein into the cell by electroporation. The TAL effector domain that binds to a specific nucleotide sequence within the target DNA can include 15 or more DNA binding repeats. The cell can be from an organism selected from the group consisting of a plant, an animal, a mammal, a human, a teleost fish, a fungus, a bacteria or a protozoa.

In another embodiment the invention includes a method for designing a sequence specific TAL effector endonuclease capable of cleaving DNA at a specific location. The method includes identifying a first unique endogenous chromosomal nucleotide sequence adjacent to a second nucleotide sequence at which it is desired to introduce a double-stranded cut; and designing a sequence specific TAL effector endonuclease comprising (a) a plurality of DNA binding repeat domains that, in combination, bind to the first unique endogenous chromosomal nucleotide sequence and which includes modifications at the 0^{th} and −1th repeat region 5′ to the RVD region, and (b) an endonuclease that generates a double-stranded cut at the second nucleotide sequence.

According to the invention, the fusion protein can be expressed in a cell, e.g., by delivering the fusion protein to the cell or by delivering a polynucleotide encoding the fusion protein to a cell, wherein the polynucleotide, if DNA, is transcribed, and an RNA molecule delivered to the cell or a transcript of a DNA molecule delivered to the cell is translated, to generate the fusion protein. Methods for polynucleotide and polypeptide delivery to cells are known in the art and are presented elsewhere in this disclosure.

Targeted mutations resulting from the aforementioned method include, but are not limited to, point mutations (i.e., conversion of a single base pair to a different base pair), substitutions (i.e., conversion of a plurality of base pairs to a different sequence of identical length), insertions or one or more base pairs, deletions of one or more base pairs and any combination of the aforementioned sequence alterations.

Methods for targeted recombination (for, e.g., alteration or replacement of a sequence in a chromosome or a region of interest in cellular chromatin) are also provided. For example, a mutant genomic sequence can be replaced by a wild-type sequence, e.g., for treatment of genetic disease or inherited disorders. In addition, a wild-type genomic sequence can be replaced by a mutant sequence, e.g., to prevent function of an oncogene product or a product of a gene involved in an inappropriate inflammatory response. Furthermore, one allele of a gene can be replaced by a different allele.

In another embodiment, the invention includes a method for designing a PthXo1 TAL effector endonuclease that does not require a thymine in the 5′ position of the target sequence. The method includes modifying the amino acid sequence of the −1 and/or 0^{th} repeats of the TAL effector domain. The method further comprises the modification of other TAL effector proteins or TAL effector-like proteins to eliminate or modify the requirement for a 5′ thymine.

The invention also includes a TAL effector endonuclease comprising an endonuclease domain and a TAL effector DNA binding domain specific for a particular DNA sequence. The TAL effector endonuclease can further include a purification tag. In a preferred embodiment, the invention includes a composition comprising a protein comprising an endonuclease domain and a TAL effector DNA binding domain (TALEN) specific for a target DNA, wherein the repeats at the 0^{th} or −1th position are modified to eliminate the need for a thymine 5′ to the target DNA binding domain. The TALEN of the composition further includes DNA binding domains wherein a plurality of DNA binding repeats contain a RVD that determines recognition of a base pair in the target DNA, wherein each DNA binding repeat is responsible for recognizing one base pair in the target DNA. The invention further includes a TALEN of, wherein the endonuclease domain is from a type II restriction endonuclease, including the type II restriction endonuclease FokI. The invention further includes a TALEN of wherein the TAL effector DNA binding domain is a Xanthomonas TAL effector, including but not limited to PthXo1, AvrXa7, PthXo3, and PthXo2. The invention also includes a TALEN wherein the TAL effector DNA binding domain is from a TAL effector-like protein of Ralstonia solanacearum.

Thus the invention comprises a method for modifying the genetic material of a cell. The method includes providing a primary cell containing a chromosomal target DNA sequence in which it is desired to have homologous recombination occur; providing a TAL effector endonuclease comprising an endonuclease domain that can cleave double stranded DNA, and a TAL effector domain comprising a plurality of TAL effector repeat sequences that, in combination, bind to a specific nucleotide sequence within the target DNA in the cell and which further includes modifications at the 0^{th} and −1th repeat region 5′ to the RVD region; and contacting the target DNA sequence with the TAL effector endonuclease in the cell such that the TAL effector endonuclease cleaves both strands of a nucleotide sequence within or adjacent to the target DNA sequence in the cell.

The invention also includes a nucleic acid sequence which encodes a TAL effector fusion protein comprising an endonuclease domain and a TAL effector DNA binding domain specific for a target DNA, which has been designed to interact with and cleave a target sequence with any nucleotide at the 5′ position, as well as an expression construct, for example a vector, that includes such a nucleic acid sequence operably linked to a promoter sequence capable of directing expression in a cell, such as a mammalian, plant, other eukaryotic cell, or a prokaryotic cell.

The invention further includes a method for providing a nucleic acid comprising a sequence homologous to at least a portion of the target DNA, such that homologous recombination occurs between the target DNA sequence and the nucleic acid. The target DNA sequence can be endogenous to the cell. The cell can be a plant cell or a mammalian cell. The contacting can include transfecting the cell with a vector comprising a TAL effector endonuclease coding sequence, and expressing the TAL effector endonuclease protein in the cell, mechanically injecting a TAL effector endonuclease protein into the cell, delivering a TAL effector endonuclease protein into the cell by means of the bacterial type III secretion system, or introducing a TAL effector endonuclease protein into the cell by electroporation. The TAL effector domain that binds to a specific nucleotide sequence within the target DNA can include 15 or more DNA binding repeats. The cell can be from an organism selected from the group consisting of a plant, an animal, a mammal, a human, a teleost fish, a fungus, a bacteria or a protozoa.

The invention further includes other TAL effector based DNA targeting. Other TAL effectors may include custom trascriptional activators, repressors, or other DNA modifiers such as dioxygenases and methylases. These effectors can be produced by the methods described herein, by substituting the effector domains of the transcriptional activators, repressors, or other DNA modifiers, such as dioxygenases and methylases, for the endonuclease domain of the TALEN proteins described. For example, a TAL effector DNA binding domain specific for a target DNA, wherein the repeats at the 0^{th} or −1th position are modified to eliminate the need for a thymine 5′ to the target DNA binding domain, and wherein the composition further includes DNA binding domains wherein a plurality of DNA binding repeats contain a RVD that determines recognition of a base pair in the target DNA, wherein each DNA binding repeat is responsible for recognizing one base pair in the target DNA, may be coupled with the activation domain of a transcriptional activator or transcription factor to produce a specifically targeted protein that can induce transcription at the selected target site. Similarly, for example, the TAL effector DNA binding domain can be customized to target a specific sequence and coupled to the effector domain of a DNA methylse to produce a protein that with methylate DNA at a specific selected site.

The invention further comprises a method for targeting the genetic material of a cell. The method includes providing a primary cell containing a chromosomal target DNA sequence; providing a TAL effector protein comprising an trascriptional activators, repressors, or other DNA modifiers effector domain, and a TAL effector domain comprising a plurality of TAL effector repeat sequences that, in combination, bind to a specific nucleotide sequence within the target DNA in the cell and which further includes modifications at the 0^{th} and −1th repeat region 5′ to the RVD region; and contacting the target DNA sequence with the TAL effector in the cell such that the trascriptional activator, repressor, or other DNA modifier effector domain of the TAL effector acts on a nucleotide sequence within or adjacent to the target DNA sequence in the cell.

The invention also includes a nucleic acid sequence which encodes a TAL effector fusion protein comprising an endonuclease domain and a TAL effector DNA binding domain specific for a target DNA, which has been designed to interact with a target sequence with any nucleotide at the 5′ position, as well as an expression construct, for example a vector, that includes such a nucleic acid sequence operably linked to a promoter sequence capable of directing expression in a cell, such as a mammalian, plant, other eukaryotic cell, or a prokaryotic cell.

In another embodiment the invention includes a method for designing a sequence specific TAL effector endonuclease capable of cleaving DNA at a specific location. The method includes identifying a first unique endogenous chromosomal nucleotide sequence adjacent to a second nucleotide sequence at which it is desired to introduce a double-stranded cut; and designing a sequence specific TAL effector endonuclease comprising (a) a plurality of DNA binding repeat domains that, in combination, bind to the first unique endogenous chromosomal nucleotide sequence and which also includes modifications at the 0^{th} and −1th repeat region 5′ to the RVD region, and (b) an endonuclease that generates a double-stranded cut at the second nucleotide sequence.

According to the invention, the fusion protein can be expressed in a cell, e.g., by delivering the fusion protein to the cell or by delivering a polynucleotide encoding the fusion protein to a cell, wherein the polynucleotide, if DNA, is transcribed, and an RNA molecule delivered to the cell or a transcript of a DNA molecule delivered to the cell is translated, to generate the fusion protein. Methods for polynucleotide and polypeptide delivery to cells are known in the art and are presented elsewhere in this disclosure.

Targeted mutations resulting from the aforementioned method include, but are not limited to, point mutations (i.e., conversion of a single base pair to a different base pair), substitutions (i.e., conversion of a plurality of base pairs to a different sequence of identical length), insertions or one or more base pairs, deletions of one or more base pairs and any combination of the aforementioned sequence alterations.

Methods for targeted recombination (for, e.g., alteration or replacement of a sequence in a chromosome or a region of interest in cellular chromatin) are also provided. For example, a mutant genomic sequence can be replaced by a wild-type sequence, e.g., for treatment of genetic disease or inherited disorders. In addition, a wild-type genomic sequence can be replaced by a mutant sequence, e.g., to prevent function of an oncogene product or a product of a gene involved in an inappropriate inflammatory response. Furthermore, one allele of a gene can be replaced by a different allele.

The invention also includes a TAL effector endonuclease comprising an endonuclease domain and a TAL effector DNA binding domain specific for a particular DNA sequence. The TAL effector endonuclease can further include a purification tag. The endonuclease domain is preferably a Type II endonuclease, more preferably ITev-I or Fok I.

### Target Sites

The disclosed methods and compositions include fusion proteins comprising a cleavage domain and a TAL effector DNA binding domain, or DNA recognition sequence in which the RVDs, by binding to a sequence in cellular chromatin (e.g., a target site or a binding site), directs the activity of the cleavage domain (or cleavage half-domain) to the vicinity of the sequence and, hence, induces cleavage in the vicinity of the target sequence. As set forth elsewhere in this disclosure, particular RVDs within a TAL binding domain can be engineered to bind to virtually any desired sequence. Accordingly, after identifying a region of interest containing a sequence at which cleavage or recombination is desired, one or more TAL effector DNA binding domains can be engineered to bind to one or more sequences in the region of interest. Expression of a fusion protein comprising a TAL effector DNA binding domain and a cleavage domain, in a cell, effects cleavage in the region of interest.

Selection of a sequence in cellular chromatin for binding by a TAL effector binding domain (e.g., a target site) can be accomplished, by any method known to those of skill in the art. For example simple visual inspection of a nucleotide sequence can be used for selection of a target site. Accordingly, any means for target site selection can be used in the claimed methods.

### Sequence-Specific Endonucleases

Sequence-specific nucleases and recombinant nucleic acids encoding the sequence-specific endonucleases are provided herein. The sequence-specific endonucleases can include TAL effector DNA binding domains and endonuclease domains. Thus, nucleic acids encoding such sequence-specific endonucleases can include a nucleotide sequence from a sequence-specific TAL effector linked to a nucleotide sequence from a nuclease.

TAL effectors are proteins of plant pathogenic bacteria that are injected by the pathogen into the plant cell, where they travel to the nucleus and function as transcription factors to turn on specific plant genes. The primary amino acid sequence of a TAL effector dictates the nucleotide sequence to which it binds. Because the relationship between the TAL amino acid sequence and the target binding site is simple, target sites can be predicted for TAL effectors, and TAL effectors also can be engineered and generated for the purpose of binding to particular nucleotide sequences.

Fused to the TAL effector-encoding nucleic acid sequences are sequences encoding a nuclease or a portion of a nuclease, typically a nonspecific cleavage domain from a type II restriction endonuclease such as FokI (Kim et al. (1996) Proc. Natl. Acad. Sci. USA 93:1156-1160). Other useful endonucleases may include, for example, HhaI, HindIII, NotI, BbvCI, EcoRI, Bg/I, and AlwI. The fact that some endonucleases (e.g., FokI) only function as dimers can be capitalized upon to enhance the target specificity of the TAL effector. For example, in some cases each FokI monomer can be fused to a TAL effector sequence that recognizes a different DNA target sequence, and only when the two recognition sites are in close proximity do the inactive monomers come together to create a functional enzyme. By requiring DNA binding to activate the nuclease, a highly site-specific restriction enzyme can be created.

Alternatively, other fusions may include trasncriptional activation domains, transcriptional repressor domains, or any of several DNA modifying domians or other protein domains, thereby creating highly site-specific effector molecules for modification or utilization of specific DNA sequences.

A sequence-specific TAL effector endonuclease as provided herein can recognize a particular sequence within a preselected target nucleotide sequence present in a cell. Thus, in some embodiments, a target nucleotide sequence can be scanned for nuclease recognition sites, and a particular nuclease can be selected based on the target sequence. In other cases, a TAL effector endonuclease can be engineered to target a particular cellular sequence. A nucleotide sequence encoding the desired TAL effector endonuclease can be inserted into any suitable expression vector, and can be linked to one or more expression control sequences. For example, a nuclease coding sequence can be operably linked to a promoter sequence that will lead to constitutive expression of the endonuclease in the species of plant to be transformed. Alternatively, an endonuclease coding sequence can be operably linked to a promoter sequence that will lead to conditional expression (e.g., expression under certain nutritional conditions).

### Cleavage Domains

The cleavage domain portion of the fusion proteins disclosed herein can be obtained from any endo- or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., S1 Nuclease; mung bean nuclease; pancreatic DNase I; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains.

“Type II restriction endonucleases” are those that cut DNA at defined positions close to or within their recognition sequences. They produce discrete restriction fragments and distinct gel banding patterns. Type II restriction endonucleases as used herein includes Type IIS and Type JIG enzymes. Exemplary Type II restriction endonucelases include FokI, HhaI, HindIII, NotI, BbvCI, EcoRI, BglI, AcuI, BcgI, and AlwI.

### TAL Effector DNA Domain-Cleavage Domain Fusions

Methods for design and construction of fusion proteins (and polynucleotides encoding same) are known to those of skill in the art. For example, methods for the design and construction of fusion protein comprising TAL proteins (and polynucleotides encoding same) are described in U.S. Pat. Nos. 6,453,242 and 6,534,261. In certain embodiments, polynucleotides encoding such fusion proteins are constructed. These polynucleotides can be inserted into a vector and the vector can be introduced into a cell (see below for additional disclosure regarding vectors and methods for introducing polynucleotides into cells).

In certain embodiments, the components of the fusion proteins are arranged such that the cleavage domain is nearest the amino terminus of the fusion protein, and the TAL domain is nearest the carboxy-terminus. This provides certain advantages such as the retention of the transcription activator activity which enables one to measure the DNA binding specificity of naturally occurring TAL or newly engineered TAL used for nuclease fusion and this orientation may give the flexibility of spacer lengths.

### Methods for Targeted Cleavage

The disclosed methods and compositions can be used to cleave DNA at a region of interest in cellular chromatin (e.g., at a desired or predetermined site in a genome, for example, in a gene, either mutant or wild-type). For such targeted DNA cleavage, TAL binding domain is engineered to bind a target site at or near the predetermined cleavage site, and a fusion protein comprising the engineered TAL binding domain and a cleavage domain is expressed in a cell. Upon binding of the TAL RVDs portion of the fusion protein to the target site, the DNA is cleaved near the target site by the cleavage domain.

For targeted cleavage using a TAL binding domain-cleavage domain fusion polypeptide, the binding site can encompass the cleavage site, or the near edge of the binding site can be 1, 2, 3, 4, 5, 6, 10, 25, 50 or more nucleotides (or any integral value between 1 and 50 nucleotides) from the cleavage site. The exact location of the binding site, with respect to the cleavage site, will depend upon the particular cleavage domain, and the length of any linker.

Thus, the methods described herein can employ an engineered TAL effector DNA binding domain fused to a cleavage domain. In these cases, the binding domain is engineered to bind to a target sequence, at or near which cleavage is desired. The fusion protein, or a polynucleotide encoding same, is introduced into a cell. Once introduced into, or expressed in, the cell, the fusion protein binds to the target sequence and cleaves at or near the target sequence. The exact site of cleavage depends on the nature of the cleavage domain and/or the presence and/or nature of linker sequences between the binding and cleavage domains. Optimal levels of cleavage can also depend on both the distance between the binding sites of the two fusion proteins (See, for example, Smith et al. (2000) Nucleic Acids Res. 28:3361-3369; Bibikova et al. (2001) Mol. Cell. Biol. 21:289-297) and the length of the ZC linker in each fusion protein.

The site at which the DNA is cleaved generally lies between the binding sites for the two fusion proteins. Double-strand breakage of DNA often results from two single-strand breaks, or “nicks,” offset by 1, 2, 3, 4, 5, 6 or more nucleotides.

As noted above, the fusion protein(s) can be introduced as polypeptides and/or polynucleotides. For example, two polynucleotides, each comprising sequences encoding one of the aforementioned polypeptides, can be introduced into a cell, and when the polypeptides are expressed and each binds to its target sequence, cleavage occurs at or near the target sequence. Alternatively, a single polynucleotide comprising sequences encoding both fusion polypeptides is introduced into a cell. Polynucleotides can be DNA, RNA or any modified forms or analogues or DNA and/or RNA.

To enhance cleavage specificity, additional compositions may also be employed in the methods described herein. For example, single cleavage domains can exhibit limited double-stranded cleavage activity.

In addition to the fusion molecules described herein, targeted replacement of a selected genomic sequence also requires the introduction of the replacement (or donor) sequence. The donor sequence can be introduced into the cell prior to, concurrently with, or subsequent to, expression of the fusion protein(s). The donor polynucleotide contains sufficient homology to a genomic sequence to support homologous recombination between it and the genomic sequence to which it bears homology. Approximately 25, 50, 100 or 200 nucleotides or more of sequence homology between a donor and a genomic sequence (or any integral value between 10 and 200 nucleotides, or more) will support homologous recombination therebetween. Donor sequences can range in length from 10 to 5,000 nucleotides (or any integral value of nucleotides therebetween) or longer. It will be readily apparent that the donor sequence is typically not identical to the genomic sequence that it replaces. For example, the sequence of the donor polynucleotide can contain one or more single base changes, insertions, deletions, inversions or rearrangements with respect to the genomic sequence, so long as sufficient homology is present to support homologous recombination. Alternatively, a donor sequence can contain a non-homologous sequence flanked by two regions of homology. Additionally, donor sequences can comprise a vector molecule containing sequences that are not homologous to the region of interest in cellular chromatin. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the donor polynucleotide.

A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

To simplify assays (e.g., hybridization, PCR, restriction enzyme digestion) for determining successful insertion of the donor sequence, certain sequence differences may be present in the donor sequence as compared to the genomic sequence. Preferably, if located in a coding region, such nucleotide sequence differences will not change the amino acid sequence, or will make silent amino acid changes (i.e., changes which do not affect the structure or function of the protein). The donor polynucleotide can optionally contain changes in sequences corresponding to the TAL effector domain binding (or recognition) sites in the region of interest, to prevent cleavage of donor sequences that have been introduced into cellular chromatin by homologous recombination.

The donor polynucleotide can be DNA or RNA, single-stranded or double-stranded and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer, or can be delivered by viruses (e.g., adenovirus, AAV).

Applicants' methods advantageously combine the powerful targeting capabilities of engineered TALs with an effector domain trasncriptional activation domain, transcriptional repressor domain, or any of several DNA modifying domians or other protein domains to specifically target DNA for repression, transcription, or modification.

For alteration of a chromosomal sequence, it is not necessary for the entire sequence of the donor to be copied into the chromosome, as long as enough of the donor sequence is copied to effect the desired sequence alteration.

In certain embodiments, a homologous chromosome can serve as the donor polynucleotide. Thus, for example, correction of a mutation in a heterozygote can be achieved by engineering fusion proteins which bind to and cleave the mutant sequence on one chromosome, but do not cleave the wild-type sequence on the homologous chromosome. The double-stranded break on the mutation-bearing chromosome stimulates a homology-based “gene conversion” process in which the wild-type sequence from the homologous chromosome is copied into the cleaved chromosome, thus restoring two copies of the wild-type sequence.

Further increases in efficiency of targeted recombination, in cells comprising fusion molecule and a donor DNA molecule, are achieved by blocking the cells in the G₂ phase of the cell cycle, when homology-driven repair processes are maximally active. Such arrest can be achieved in a number of ways. For example, cells can be treated with e.g., drugs, compounds and/or small molecules which influence cell-cycle progression so as to arrest cells in G₂ phase. Exemplary molecules of this type include, but are not limited to, compounds which affect microtubule polymerization (e.g., vinblastine, nocodazole, Taxol), compounds that interact with DNA (e.g., cis-platinum(II) diamine dichloride, Cisplatin, doxorubicin) and/or compounds that affect DNA synthesis (e.g., thymidine, hydroxyurea, L-mimosine, etoposide, 5-fluorouracil). Additional increases in recombination efficiency are achieved by the use of histone deacetylase (HDAC) inhibitors (e.g., sodium butyrate, trichostatin A) which alter chromatin structure to make genomic DNA more accessible to the cellular recombination machinery.

Additional methods for cell-cycle arrest include overexpression of proteins which inhibit the activity of the CDK cell-cycle kinases, for example, by introducing a cDNA encoding the protein into the cell or by introducing into the cell an engineered TAL effector which activates expression of the gene encoding the protein. Cell-cycle arrest is also achieved by inhibiting the activity of cyclins and CDKs, for example, using RNAi methods (e.g., U.S. Pat. No. 6,506,559) or by introducing into the cell an engineered TAL effector which represses expression of one or more genes involved in cell-cycle progression such as, for example, cyclin and/or CDK genes. See, e.g., U.S. Pat. No. 6,534,261 for methods for the synthesis of engineered TAL proteins for regulation of gene expression.

Methods to Screen for Cellular Factors that Facilitate Homologous Recombination

Since homologous recombination is a multi-step process requiring the modification of DNA ends and the recruitment of several cellular factors into a protein complex, the addition of one or more exogenous factors, along with donor DNA and vectors encoding TAL-cleavage domain fusions, can be used to facilitate targeted homologous recombination. An exemplary method for identifying such a factor or factors employs analyses of gene expression using microarrays (e.g., Affymetrix Gene Chip® arrays) to compare the mRNA expression patterns of different cells. For example, cells that exhibit a higher capacity to stimulate double strand break-driven homologous recombination in the presence of donor DNA and TAL-cleavage domain fusions, either unaided or under conditions known to increase the level of gene correction, can be analyzed for their gene expression patterns compared to cells that lack such capacity. Genes that are upregulated or downregulated in a manner that directly correlates with increased levels of homologous recombination are thereby identified and can be cloned into any one of a number of expression vectors. These expression constructs can be co-transfected along with TAL-cleavage domain fusions and donor constructs to yield improved methods for achieving high-efficiency homologous recombination.

### Expression Vectors

A nucleic acid encoding one or more fusion proteins can be cloned into a vector for transformation into prokaryotic or eukaryotic cells for replication and/or expression. Vectors can be prokaryotic vectors, e.g., plasmids, or shuttle vectors, insect vectors, or eukaryotic vectors. A nucleic acid encoding a TAL effector binding domain can also be cloned into an expression vector, for administration to a plant cell, animal cell, preferably a mammalian cell or a human cell, fungal cell, bacterial cell, or protozoal cell.

To obtain expression of a cloned gene or nucleic acid, sequences encoding a fusion protein are typically subcloned into an expression vector that contains a promoter to direct transcription.

Promoters are involved in recognition and binding of RNA polymerase and other proteins to initiate and modulate transcription. To bring a coding sequence under the control of a promoter, it typically is necessary to position the translation initiation site of the translational reading frame of the polypeptide between one and about fifty nucleotides downstream of the promoter. A promoter can, however, be positioned as much as about 5,000 nucleotides upstream of the translation start site, or about 2,000 nucleotides upstream of the transcription start site. A promoter typically comprises at least a core (basal) promoter. A promoter also may include at least one control element such as an upstream element. Such elements include upstream activation regions (UARs) and, optionally, other DNA sequences that affect transcription of a polynucleotide such as a synthetic upstream element.

The choice of promoters to be included depends upon several factors, including, but not limited to, efficiency, selectability, inducibility, desired expression level, and cell or tissue specificity. For example, tissue-, organ- and cell-specific promoters that confer transcription only or predominantly in a particular tissue, organ, and cell type, respectively, can be used. In some embodiments, promoters specific to vegetative tissues such as the stem, parenchyma, ground meristem, vascular bundle, cambium, phloem, cortex, shoot apical meristem, lateral shoot meristem, root apical meristem, lateral root meristem, leaf primordium, leaf mesophyll, or leaf epidermis can be suitable regulatory regions. In some embodiments, promoters that are essentially specific to seeds (“seed-preferential promoters”) can be useful. Seed-specific promoters can promote transcription of an operably linked nucleic acid in endosperm and cotyledon tissue during seed development. Alternatively, constitutive promoters can promote transcription of an operably linked nucleic acid in most or all tissues of a plant, throughout plant development. Other classes of promoters include, but are not limited to, inducible promoters, such as promoters that confer transcription in response to external stimuli such as chemical agents, developmental stimuli, or environmental stimuli.

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

Non-limiting examples of promoters that can be included in the nucleic acid constructs provided herein include the cauliflower mosaic virus (CaMV) 35S transcription initiation region, the 1′ or 2′ promoters derived from T-DNA of Agrobacterium tumefaciens, promoters from a maize leaf-specific gene described by Busk ((1997) Plant J 11:1285-1295), kn1-related genes from maize and other species, and transcription initiation regions from various plant genes such as the maize ubiquitin-1 promoter.

A 5′ untranslated region (UTR) is transcribed, but is not translated, and lies between the start site of the transcript and the translation initiation codon and may include the +1 nucleotide. A 3′ UTR can be positioned between the translation termination codon and the end of the transcript. UTRs can have particular functions such as increasing mRNA message stability or translation attenuation. Examples of 3′ UTRs include, but are not limited to polyadenylation signals and transcription termination sequences. A polyadenylation region at the 3′-end of a coding region can also be operably linked to a coding sequence. The polyadenylation region can be derived from the natural gene, from various other plant genes, or from an Agrobacterium T-DNA.

The vectors provided herein also can include, for example, origins of replication, and/or scaffold attachment regions (SARs). In addition, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag tag (Kodak, New Haven, Conn.) sequences typically are expressed as a fusion with the encoded polypeptide. Such tags can be inserted anywhere within the polypeptide, including at either the carboxyl or amino terminus.

It will be understood that more than one regulatory region may be present in a recombinant polynucleotide, e.g., introns, enhancers, upstream activation regions, and inducible elements.

Recombinant nucleic acid constructs can include a polynucleotide sequence inserted into a vector suitable for transformation of cells (e.g., plant cells or animal cells). Recombinant vectors can be made using, for example, standard recombinant DNA techniques (see, e.g., Sambrook et al. (1989) Molecular Cloning: A Laboratory Manual, 2nd ed., Cold Spring Harbor Laboratory, Cold Spring Harbor, N.Y.).

Suitable bacterial and eukaryotic promoters are well known in the art and described, e.g., in Sambrook et al., Molecular Cloning, A Laboratory Manual (2nd ed. 1989; 3rd ed., 2001); Kriegler, Gene Transfer and Expression: A Laboratory Manual (1990); and Current Protocols in Molecular Biology (Ausubel et al., supra. Bacterial expression systems for expressing the ZFP are available in, e.g., E. coli, Bacillus sp., and Salmonella (Palva et al., Gene 22:229-235 (1983)). Kits for such expression systems are commercially available. Eukaryotic expression systems for mammalian cells, yeast, and insect cells are well known by those of skill in the art and are also commercially available.

The promoter used to direct expression of a TAL-cleavage domain fusion protein-encoding nucleic acid depends on the particular application. For example, a strong constitutive promoter is typically used for expression and purification of TAL-cleavage domain fusion proteins. In contrast, when a TAL-cleavage domain fusion protein is administered in vivo for gene regulation, either a constitutive or an inducible promoter is used, depending on the particular use of the TAL-cleavage domain fusion protein. In addition, a preferred promoter for administration of a TAL-cleavage domain fusion protein can be a weak promoter, such as HSV TK or a promoter having similar activity. The promoter typically can also include elements that are responsive to transactivation, e.g., hypoxia response elements, Gal4 response elements, lac repressor response element, and small molecule control systems such as tet-regulated systems and the RU-486 system (see, e.g., Gossen & Bujard, PNAS 89:5547 (1992); Oligino et al., Gene Ther. 5:491-496 (1998); Wang et al., Gene Ther. 4:432-441 (1997); Neering et al., Blood 88:1147-1155 (1996); and Rendahl et al., Nat. Biotechnol. 16:757-761 (1998)). The MNDU3 promoter can also be used, and is preferentially active in CD34+ hematopoietic stem cells.

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to a nucleic acid sequence encoding the TAL-cleavage domain fusion protein and signals required, e.g., for efficient polyadenylation of the transcript, transcriptional termination, ribosome binding sites, or translation termination. Additional elements of the cassette may include, e.g., enhancers, and heterologous splicing signals.

The particular expression vector used to transport the genetic information into the cell is selected with regard to the intended use of the TAL-cleavage domain fusion protein, e.g., expression in plants, animals, bacteria, fungus, protozoa, etc. (see expression vectors described below). Standard bacterial expression vectors include plasmids such as pBR322-based plasmids, pSKF, pET23D, and commercially available fusion expression systems such as GST and LacZ. An exemplary fusion protein is the maltose binding protein, “MBP.” Such fusion proteins are used for purification of the TAL-cleavage domain fusion protein. Epitope tags can also be added to recombinant proteins to provide convenient methods of isolation, for monitoring expression, and for monitoring cellular and subcellular localization, e.g., c-myc or FLAG.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with a TAL-cleavage domain fusion protein encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce plant, bacterial, mammalian, yeast or insect cell lines that express large quantities of protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well known procedures for introducing foreign nucleotide sequences into host cells may be used. These include the use of calcium phosphate transfection, polybrene, protoplast fusion, electroporation, ultrasonic methods (e.g., sonoporation), liposomes, microinjection, naked DNA, plasmid vectors, viral vectors, both episomal and integrative, and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the protein of choice.

### Nucleic Acids Encoding Fusion Proteins and Delivery to Cells

Conventional viral and non-viral based gene transfer methods can be used to introduce nucleic acids encoding engineered TAL-cleavage domain fusion proteins in animal cells (e.g., mammalian cells) and target tissues. Such methods can also be used to administer nucleic acids encoding TAL-cleavage domain fusion proteins to cells in vitro. In certain embodiments, nucleic acids encoding TAL-cleavage domain fusion proteins are administered for in vivo or ex vivo gene therapy uses. Non-viral vector delivery systems include DNA plasmids, naked nucleic acid, and nucleic acid complexed with a delivery vehicle such as a liposome or poloxamer. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds) (1995); and Yu et al., Gene Therapy 1: 13-26 (1994).

Methods of non-viral delivery of nucleic acids encoding engineered TAL-cleavage domain fusion proteins include electroporation, lipofection, microinjection, biolistics, virosomes, liposomes, immunoliposomes, polycation or lipid:nucleic acid conjugates, naked DNA, artificial virions, and agent-enhanced uptake of DNA. Sonoporation using, e.g., the Sonitron 2000 system (Rich-Mar) can also be used for delivery of nucleic acids.

Additional exemplary nucleic acid delivery systems include those provided by Amaxa Biosystems (Cologne, Germany), Maxcyte, Inc. (Rockville, Md.) and BTX Molecular Delivery Systems (Holliston, Mass.).

The use of RNA or DNA viral based systems for the delivery of nucleic acids encoding engineered TAL-cleavage domain fusion proteins take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of TAL-cleavage domain fusion proteins include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

In applications in which transient expression of a TAL-cleavage domain fusion protein fusion protein is preferred, adenoviral based systems can be used. Adenoviral based vectors are capable of very high transduction efficiency in many cell types and do not require cell division. With such vectors, high titer and high levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Replication-deficient recombinant adenoviral vectors (Ad) can be produced at high titer and readily infect a number of different cell types. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including nondividing, differentiated cells such as those found in liver, kidney and muscle. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:1 5-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

Packaging cells are used to form virus particles that are capable of infecting a host cell. Such cells include 293 cells, which package adenovirus, and .psi.2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al., Proc. Natl. Acad. Sci. USA 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a ZFP nucleic acid (gene or cDNA), and re-infused back into the subject organism (e.g., patient). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba et al., J. Exp. Med. 176:1693-1702 (1992)).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and lad (differentiated antigen presenting cells) (see Inaba et al., J. Exp. Med. 176:1693-1702 (1992)).

Vectors (e.g., retroviruses, adenoviruses, liposomes, etc.) containing therapeutic TAL-cleavage domain fusion protein nucleic acids can also be administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

With further respect to plants, the polynucleotides and vectors described herein can be used to transform a number of monocotyledonous and dicotyledonous plants and plant cell systems, including dicots such as safflower, alfalfa, soybean, coffee, amaranth, rapeseed (high erucic acid and canola), peanut or sunflower, as well as monocots such as oil palm, sugarcane, banana, sudangrass, com, wheat, rye, barley, oat, rice, millet, or sorghum. Also suitable are gymnosperms such as fir and pine.

Thus, the methods described herein can be utilized with dicotyledonous plants belonging, for example, to the orders Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violates, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales. The methods described herein also can be utilized with monocotyledonous plants such as those belonging to the orders Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales, or with plants belonging to Gymnospermae, e.g., Pinales, Ginkgoales, Cycadales and Gnetales.

The methods can be used over a broad range of plant species, including species from the dicot genera Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Trifolium, Trigonella, Vicia, Vinca, Vilis, and Vigna; the monocot genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, and Zea; or the gymnosperm genera Abies, Cunninghamia, Picea, Pinus, and Pseudotsuga.

A transformed cell, callus, tissue, or plant can be identified and isolated by selecting or screening the engineered cells for particular traits or activities, e.g., those encoded by marker genes or antibiotic resistance genes. Such screening and selection methodologies are well known to those having ordinary skill in the art. In addition, physical and biochemical methods can be used to identify transformants. These include Southern analysis or PCR amplification for detection of a polynucleotide; Northern blots, S1 RNase protection, primer-extension, or RT-PCR amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are well known. Polynucleotides that are stably incorporated into plant cells can be introduced into other plants using, for example, standard breeding techniques.

DNA constructs may be introduced into the genome of a desired plant host by a variety of conventional techniques. For reviews of such techniques see, for example, Weissbach & Weissbach Methods for Plant Molecular Biology (1988, Academic Press, N.Y.) Section VIII, pp. 421-463; and Grierson & Corey, Plant Molecular Biology (1988, 2d Ed.), Blackie, London, Ch. 7-9. For example, the DNA construct may be introduced directly into the genomic DNA of the plant cell using techniques such as electroporation and microinjection of plant cell protoplasts, or the DNA constructs can be introduced directly to plant tissue using biolistic methods, such as DNA particle bombardment (see, e.g., Klein et al (1987) Nature 327:70-73). Alternatively, the DNA constructs may be combined with suitable T-DNA flanking regions and introduced into a conventional Agrobacterium tumefaciens host vector. Agrobacterium tumefaciens-mediated transformation techniques, including disarming and use of binary vectors, are well described in the scientific literature. See, for example Horsch et al (1984) Science 233:496-498, and Fraley et al (1983) Proc. Nat'l. Acad. Sci. USA 80:4803. The virulence functions of the Agrobacterium tumefaciens host will direct the insertion of the construct and adjacent marker into the plant cell DNA when the cell is infected by the bacteria using binary T DNA vector (Bevan (1984) Nuc. Acid Res. 12:8711-8721) or the co-cultivation procedure (Horsch et al (1985) Science 227:1229-1231). Generally, the Agrobacterium transformation system is used to engineer dicotyledonous plants (Bevan et al (1982) Ann. Rev. Genet 16:357-384; Rogers et al (1986) Methods Enzymol. 118:627-641). The Agrobacterium transformation system may also be used to transform, as well as transfer, DNA to monocotyledonous plants and plant cells. See Hernalsteen et al (1984) EMBO J 3:3039-3041; Hooykass-Van Slogteren et al (1984) Nature 311:763-764; Grimsley et al (1987) Nature 325:1677-179; Boulton et al (1989) Plant Mol. Biol. 12:31-40; and Gould et al (1991) Plant Physiol. 95:426-434.

Alternative gene transfer and transformation methods include, but are not limited to, protoplast transformation through calcium-, polyethylene glycol (PEG)- or electroporation-mediated uptake of naked DNA (see Paszkowski et al. (1984) EMBO J 3:2717-2722, Potrykus et al. (1985) Molec. Gen. Genet. 199:169-177; Fromm et al. (1985) Proc. Nat. Acad. Sci. USA 82:5824-5828; and Shimamoto (1989) Nature 338:274-276) and electroporation of plant tissues (D'Halluin et al. (1992) Plant Cell 4:1495-1505). Additional methods for plant cell transformation include microinjection, silicon carbide mediated DNA uptake (Kaeppler et al. (1990) Plant Cell Reporter 9:415-418), and microprojectile bombardment (see Klein et al. (1988) Proc. Nat. Acad. Sci. USA 85:4305-4309; and Gordon-Kamm et al. (1990) Plant Cell 2:603-618).

The disclosed methods and compositions can be used to insert exogenous sequences into a predetermined location in a plant cell genome. This is useful inasmuch as expression of an introduced transgene into a plant genome depends critically on its integration site. Accordingly, genes encoding, e.g., nutrients, antibiotics or therapeutic molecules can be inserted, by targeted recombination, into regions of a plant genome favorable to their expression.

Transformed plant cells which are produced by any of the above transformation techniques can be cultured to regenerate a whole plant which possesses the transformed genotype and thus the desired phenotype. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans, et al., “Protoplasts Isolation and Culture” in Handbook of Plant Cell Culture, pp. 124-176, Macmillian Publishing Company, New York, 1983; and Binding, Regeneration of Plants, Plant Protoplasts, pp. 21-73, CRC Press, Boca Raton, 1985. Regeneration can also be obtained from plant callus, explants, organs, pollens, embryos or parts thereof. Such regeneration techniques are described generally in Klee et al (1987) Ann. Rev. of Plant Phys. 38:467-486.

Nucleic acids introduced into a plant cell can be used to confer desired traits on essentially any plant. A wide variety of plants and plant cell systems may be engineered for the desired physiological and agronomic characteristics described herein using the nucleic acid constructs of the present disclosure and the various transformation methods mentioned above. In preferred embodiments, target plants and plant cells for engineering include, but are not limited to, those monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). Thus, the disclosed methods and compositions have use over a broad range of plants, including, but not limited to, species from the genera Asparagus, Avena, Brassica, Citrus, Citrullus, Capsicum, Cucurbita, Daucus, Glycine, Hordeum, Lactuca, Lycopersicon, Malus, Manihot, Nicotiana, Oryza, Persea, Pisum, Pyrus, Prunus, Raphanus, Secale, Solanum, Sorghum, Triticum, Vitis, Vigna, and Zea. One of skill in the art will recognize that after the expression cassette is stably incorporated in transgenic plants and confirmed to be operable, it can be introduced into other plants by sexual crossing. Any of a number of standard breeding techniques can be used, depending upon the species to be crossed.

A transformed plant cell, callus, tissue or plant may be identified and isolated by selecting or screening the engineered plant material for traits encoded by the marker genes present on the transforming DNA. For instance, selection may be performed by growing the engineered plant material on media containing an inhibitory amount of the antibiotic or herbicide to which the transforming gene construct confers resistance. Further, transformed plants and plant cells may also be identified by screening for the activities of any visible marker genes (e.g., the β-glucuronidase, luciferase, B or C1 genes) that may be present on the recombinant nucleic acid constructs. Such selection and screening methodologies are well known to those skilled in the art.

Physical and biochemical methods also may be used to identify plant or plant cell transformants containing inserted gene constructs. These methods include but are not limited to: 1) Southern analysis or PCR amplification for detecting and determining the structure of the recombinant DNA insert; 2) Northern blot, S1 RNase protection, primer-extension or reverse transcriptase-PCR amplification for detecting and examining RNA transcripts of the gene constructs; 3) enzymatic assays for detecting enzyme or ribozyme activity, where such gene products are encoded by the gene construct; 4) protein gel electrophoresis, Western blot techniques, immunoprecipitation, or enzyme-linked immunoassays, where the gene construct products are proteins. Additional techniques, such as in situ hybridization, enzyme staining, and immunostaining, also may be used to detect the presence or expression of the recombinant construct in specific plant organs and tissues. The methods for doing all these assays are well known to those skilled in the art.

Effects of gene manipulation using the methods disclosed herein can be observed by, for example, northern blots of the RNA (e.g., mRNA) isolated from the tissues of interest. Typically, if the amount of mRNA has increased, it can be assumed that the corresponding endogenous gene is being expressed at a greater rate than before. Other methods of measuring gene and/or CYP74B activity can be used. Different types of enzymatic assays can be used, depending on the substrate used and the method of detecting the increase or decrease of a reaction product or by-product. In addition, the levels of and/or CYP74B protein expressed can be measured immunochemically, i.e., ELISA, RIA, EIA and other antibody based assays well known to those of skill in the art, such as by electrophoretic detection assays (either with staining or western blotting). The transgene may be selectively expressed in some tissues of the plant or at some developmental stages, or the transgene may be expressed in substantially all plant tissues, substantially along its entire life cycle. However, any combinatorial expression mode is also applicable.

The present disclosure also encompasses seeds of the transgenic plants described above wherein the seed has the transgene or gene construct. The present disclosure further encompasses the progeny, clones, cell lines or cells of the transgenic plants described above wherein said progeny, clone, cell line or cell has the transgene or gene construct.

### Delivery Vehicles

An important factor in the administration of polypeptide compounds, such as TAL-cleavage domain fusion protein, is ensuring that the polypeptide has the ability to traverse the plasma membrane of a cell, or the membrane of an intra-cellular compartment such as the nucleus. Cellular membranes are composed of lipid-protein bilayers that are freely permeable to small, nonionic lipophilic compounds and are inherently impermeable to polar compounds, macromolecules, and therapeutic or diagnostic agents. However, proteins and other compounds such as liposomes have been described, which have the ability to translocate polypeptides such as TAL-cleavage domain fusion proteins across a cell membrane.

For example, “membrane translocation polypeptides” have amphiphilic or hydrophobic amino acid subsequences that have the ability to act as membrane-translocating carriers. In one embodiment, homeodomain proteins have the ability to translocate across cell membranes. The shortest internalizable peptide of a homeodomain protein, Antennapedia, was found to be the third helix of the protein, from amino acid position 43 to 58 (see, e.g., Prochiantz, Current Opinion in Neurobiology 6:629-634 (1996)). Another subsequence, the h (hydrophobic) domain of signal peptides, was found to have similar cell membrane translocation characteristics (see, e.g., Lin et al., J. Biol. Chem. 270:14255-14258 (1995)).

Examples of peptide sequences which can be linked to a protein, for facilitating uptake of the protein into cells, include, but are not limited to: an 11 amino acid peptide of the tat protein of HIV; a 20 residue peptide sequence which corresponds to amino acids 84-103 of the p16 protein (see Fahraeus et al., Current Biology 6:84 (1996)); the third helix of the 60-amino acid long homeodomain of Antennapedia (Derossi et al., J. Biol. Chem. 269:10444 (1994)); the h region of a signal peptide such as the Kaposi fibroblast growth factor (K-FGF) h region (Lin et al., supra); or the VP22 translocation domain from HSV (Elliot & O'Hare, Cell 88:223-233 (1997)). Other suitable chemical moieties that provide enhanced cellular uptake may also be chemically linked to ZFPs. Membrane translocation domains (i.e., internalization domains) can also be selected from libraries of randomized peptide sequences. See, for example, Yeh et al. (2003) Molecular Therapy 7(5):S461, Abstract #1191.

Toxin molecules also have the ability to transport polypeptides across cell membranes. Often, such molecules (called “binary toxins”) are composed of at least two parts: a translocation/binding domain or polypeptide and a separate toxin domain or polypeptide. Typically, the translocation domain or polypeptide binds to a cellular receptor, and then the toxin is transported into the cell. Several bacterial toxins, including Clostridium perfringens iota toxin, diphtheria toxin (DT), Pseudomonas exotoxin A (PE), pertussis toxin (PT), Bacillus anthracis toxin, and pertussis adenylate cyclase (CYA), have been used to deliver peptides to the cell cytosol as internal or amino-terminal fusions (Arora et al., J. Biol. Chem., 268:3334-3341 (1993); Perelle et al., Infect. Immun., 61:5147-5156 (1993); Stennark et al. J. Cell Biol. 113:1025-1032 (1991); Donnelly et al., PNAS 90:3530-3534 (1993); Carbonetti et al., Abstr. Annu Meet. Am. Soc. Microbiol. 95:295 (1995); Sebo et al. Infect. Immun. 63:3851-3857 (1995); Klimpel et al. PNAS U.S.A. 89:10277-10281 (1992); and Novak et al., J. Biol. Chem. 267:17186-17193 1992)).

Such peptide sequences can be used to translocate TAL-cleavage domain fusion proteins across a cell membrane. TAL-cleavage domain fusion proteins can be conveniently fused to or derivatized with such sequences. Typically, the translocation sequence is provided as part of a fusion protein. Optionally, a linker can be used to link the TAL-cleavage domain fusion protein and the translocation sequence. Any suitable linker can be used, e.g., a peptide linker.

The TAL-cleavage domain fusion protein can also be introduced into an animal cell, preferably a mammalian cell, via a liposomes and liposome derivatives such as immunoliposomes. The term “liposome” refers to vesicles comprised of one or more concentrically ordered lipid bilayers, which encapsulate an aqueous phase. The aqueous phase typically contains the compound to be delivered to the cell,

The liposome fuses with the plasma membrane, thereby releasing the drug into the cytosol. Alternatively, the liposome is phagocytosed or taken up by the cell in a transport vesicle. Once in the endosome or phagosome, the liposome either degrades or fuses with the membrane of the transport vesicle and releases its contents.

In current methods of drug delivery via liposomes, the liposome ultimately becomes permeable and releases the encapsulated compound (in this case, a TAL-cleavage domain fusion protein) at the target tissue or cell. For systemic or tissue specific delivery, this can be accomplished, for example, in a passive manner wherein the liposome bilayer degrades over time through the action of various agents in the body. Alternatively, active drug release involves using an agent to induce a permeability change in the liposome vesicle. Liposome membranes can be constructed so that they become destabilized when the environment becomes acidic near the liposome membrane (see, e.g., PNAS 84:7851 (1987); Biochemistry 28:908 (1989)). When liposomes are endocytosed by a target cell, for example, they become destabilized and release their contents. This destabilization is termed fusogenesis. Dioleoylphosphatidylethanolamine (DOPE) is the basis of many “fusogenic” systems.

The disclosed methods for targeted recombination can be used to replace any genomic sequence with a homologous, non-identical sequence. For example, a mutant genomic sequence can be replaced by its wild-type counterpart, thereby providing methods for treatment of e.g., genetic disease, inherited disorders, cancer, and autoimmune disease. In like fashion, one allele of a gene can be replaced by a different allele using the methods of targeted recombination disclosed herein. Exemplary genetic diseases include, but are not limited to, achondroplasia, achromatopsia, acid maltase deficiency, adenosine deaminase deficiency (OMIM No. 102700), adrenoleukodystrophy, aicardi syndrome, alpha-1 antitrypsin deficiency, alpha-thalassemia, androgen insensitivity syndrome, apert syndrome, arrhythmogenic right ventricular, dysplasia, ataxia telangictasia, barth syndrome, beta-thalassemia, blue rubber bleb nevus syndrome, canavan disease, chronic granulomatous diseases (CGD), cri du chat syndrome, cystic fibrosis, dercum's disease, ectodermal dysplasia, fanconi anemia, fibrodysplasia ossificans progressive, fragile X syndrome, galactosemis, Gaucher's disease, generalized gangliosidoses (e.g., GM1), hemochromatosis, the hemoglobin C mutation in the 6.sup.th codon of beta-globin (HbC), hemophilia, Huntington's disease, Hurler Syndrome, hypophosphatasia, Kinefleter syndrome, Krabbes Disease, Langer-Giedion Syndrome, leukocyte adhesion deficiency (LAD, OMIM No. 116920), leukodystrophy, long QT syndrome, Marfan syndrome, Moebius syndrome, mucopolysaccharidosis (MPS), nail patella syndrome, nephrogenic diabetes insipdius, neurofibromatosis, Neimann-Pick disease, osteogenesis imperfecta, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, X-linked lymphoproliferative syndrome (XLP, OMIM No. 308240).

Additional exemplary diseases that can be treated by targeted DNA cleavage and/or homologous recombination include acquired immunodeficiencies, lysosomal storage diseases (e.g., Gaucher's disease, GM1, Fabry disease and Tay-Sachs disease), mucopolysaccahidosis (e.g. Hunter's disease, Hurler's disease), hemoglobinopathies (e.g., sickle cell diseases, HbC, α-thalassemia, β-thalassemia) and hemophilias.

In certain cases, alteration of a genomic sequence in a pluripotent cell (e.g., a hematopoietic stem cell) is desired. Methods for mobilization, enrichment and culture of hematopoietic stem cells are known in the art. See for example, U.S. Pat. Nos. 5,061,620; 5,681,559; 6,335,195; 6,645,489 and 6,667,064. Treated stem cells can be returned to a patient for treatment of various diseases including, but not limited to, SCID and sickle-cell anemia.

In many of these cases, a region of interest comprises a mutation, and the donor polynucleotide comprises the corresponding wild-type sequence. Similarly, a wild-type genomic sequence can be replaced by a mutant sequence, if such is desirable. For example, overexpression of an oncogene can be reversed either by mutating the gene or by replacing its control sequences with sequences that support a lower, non-pathologic level of expression. As another example, the wild-type allele of the ApoAI gene can be replaced by the ApoAI Milano allele, to treat atherosclerosis. Indeed, any pathology dependent upon a particular genomic sequence, in any fashion, can be corrected or alleviated using the methods and compositions disclosed herein.

Targeted cleavage and targeted recombination can also be used to alter non-coding sequences (e.g., regulatory sequences such as promoters, enhancers, initiators, terminators, splice sites) to alter the levels of expression of a gene product. Such methods can be used, for example, for therapeutic purposes, functional genomics and/or target validation studies.

The following examples are intended to further illustrate and in no way limit the invention.

### Example 1

### The Cocrystal Structure of TAL Effector PthXo1 Bound to its DNA Target

TAL effector-DNA recognition is mediated by tandem, 33 to 35 residue repeats that specify nucleotides via unique repeat variable diresidues (RVDs). The structure of the PthXo1 TAL effector bound to its DNA target was determined using high-throughput computational structure prediction, validated by heavy-atom derivatization. Each repeat forms a left-handed, two-helix bundle linked by an RVD-containing loop. The repeats self-associate to form a right-handed superhelix that wraps around two turns of a largely unperturbed DNA duplex. The first RVD residue forms a stabilizing contact with the protein backbone, while the second makes a base-specific contact to the DNA sense strand. Two degenerate N-terminal repeats also interact with the DNA. Containing seven RVD types and two non-canonical associations, the structure illustrates the basis of TAL effector-DNA recognition.

TAL effectors are proteins injected into plant cells by pathogens in the bacterial genus Xanthomonas, that directly and specifically activate the expression of individual plant genes during infection. These trans-kingdom transcription factors bind to host gene promoter sequences specified by a central domain in each protein that contains a variable number of tandem, polymorphic, 33 to 35 amino acid repeats, followed by a truncated “half repeat.” Each of the repeats and the half repeat preferentially associates with one of the four nucleotides in a common DNA strand to define the TAL effector binding site. Disease pressure has selected for promoter variants and other host defense mechanisms to disarm TAL effector-wielding pathogens. The recombinogenic potential of the TAL effector repeats is postulated to be the basis for pathogen coevolution that has resulted in a broad diversity of these proteins in Xanthomonas populations. Other organisms, for example Ralstonia solanacearum, possess TAL effector-like proteins that may function similarly to Xanthomonas TAL effectors.

The nucleotide specificity of individual TAL effector repeats is encoded in a polymorphic pair of adjacent residues (located at positions 12 and 13) called the repeat-variable diresidue (RVD). More than 20 unique RVD sequences have been observed in TAL effectors, but just seven account for nearly 90% of the total repeats contained in these proteins. Five of the seven, HD, NG, NI, NN, and NS, have been computationally and experimentally shown to respectively specify C, T, A, G/A, or any base. The remaining two, ‘N’ (missing the second residue) and HG, have been predicted (based on association frequencies) to respectively specify C/T and T. These simple relationships make it possible to derive the target specificity for existing TAL effectors. It is also straightforward to engineer novel TAL effectors containing custom assortments of repeats that bind DNA sequences of choice. Consequently, TAL effectors have received much attention as DNA targeting tools. In particular, they have been customized for targeted gene activation, both in plants and in animal cells, and they have been used in combination with a tethered endonuclease domain derived from the FokI restriction enzyme to generate targeted DNA double strand breaks for genome engineering in various organisms.

TAL effector-DNA specificity is not absolute. Alignments of the repeat arrays of most naturally occurring TAL effectors with their naturally occurring cognate binding sites reveal apparent mismatches. Experimental evidence to date supports the conclusion that the relative frequencies with which different RVDs associate with different nucleotides reflect the relative affinities that collectively confer specificity of the overall protein-DNA interaction. However, contributions of individual RVD-nucleotide associations to overall affinity have not been measured biochemically.

TAL effectors display a highly modular architecture, in which the repeat region is flanked by an N terminal region containing sequences that direct the protein through the bacterial type III secretion pathway and a C-terminal region that contains nuclear localization signals, an acidic activation domain, and presumably additional sites for interaction with the plant transcriptional machinery. Failure of a TAL effector repeat region by itself to target a fused endonuclease for DNA cleavage suggested that additional portions of the protein outside the repeat region are required for DNA binding, but the minimal DNA binding domain has not yet been precisely defined. Functional binding sites observed in nature are uniformly preceded by a T, which in at least one case was shown to be required for gene activation. As well, the amino acid sequence immediately preceding the TAL effector repeat region bears some similarity to the repeat consensus. It has therefore been suggested that this part of the protein, termed the ‘0^{th}’ repeat, may participate in DNA binding by forming a cryptic repeat structure that interacts with and specifies the T, though the sequence does not display an RVD found in the canonical repeats.

A recent NMR structural study of a polypeptide corresponding to 1.5 repeats of TAL effector PthA, and an accompanying SAXS study of the entire protein, indicated that an isolated TAL effector repeat is largely helical and may display similarity to a tetratricopeptide (TPR) fold, and that the full-length TAL protein displays an elongated shape that undergoes considerable compaction upon DNA binding. However, it is unclear to what extent the structure of TAL effector repeats that are found in the context of the entire protein might differ from an isolated repeat, and the likely manner in which individual repeats are associated with contiguous DNA base pairs in an effector binding site remains unresolved.

Here, we describe the three dimensional structure of the core DNA binding region of TAL effector PthXo1 of Xanthomonas oryzae, spanning TAL repeats 1 to 22 and 70 additional N-terminal residues, in complex with the DNA sequence, including the 5′ thymine, corresponding to the 25 base pair binding site for the PthXo1 TAL effector found in the promoter of the rice Os8N3 gene. The structure was determined using high-throughput computational strategy consisting of large-scale de novo structure predictions of the bound TAL effector/DNA complex and molecular replacement (MR) analyses, and subsequent validation using heavy atom anomalous difference signals and model-free features in electron density maps. The structure demonstrates that the repeats form a tightly packed, right-handed superhelix that intimately tracks the major groove around two full turns of a largely unperturbed B-DNA. Individual repeats each form a left-handed, two-helix bundle that projects a connecting loop containing the RVD deep into the DNA major groove. The first residue of each RVD forms a stabilizing contact with the protein backbone, while the second makes a base-specific contact to the DNA sense strand. Residues 219 to 280 form two additional degenerate repeat motifs (including the previously postulated 0th repeat) that make additional contacts to the 5′ thymine at the beginning of the DNA target sequence. The structure contains six of the most common repeat types in association with their preferred nucleotides, as well as two in association with a mismatched nucleotide. Most associations occur multiple times in the complex, and reveal a striking degree of conformational homogeneity across repeats. Together the structural observations explain TAL effector-DNA recognition and provide a platform for future prediction of mismatch consequences as well as the specificities of rare and novel RVDs.

### Results and Discussion

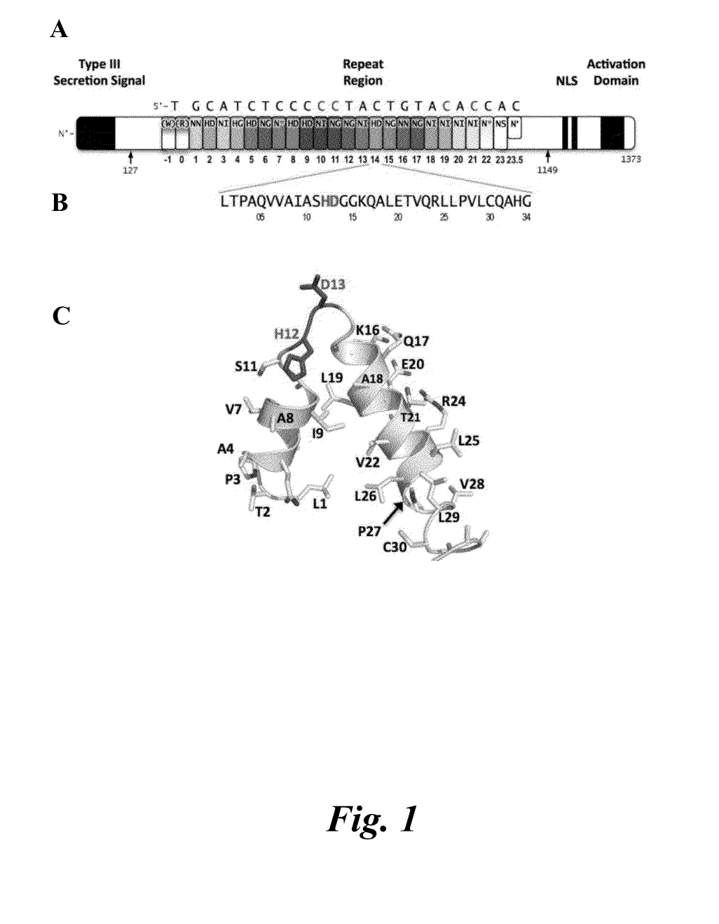

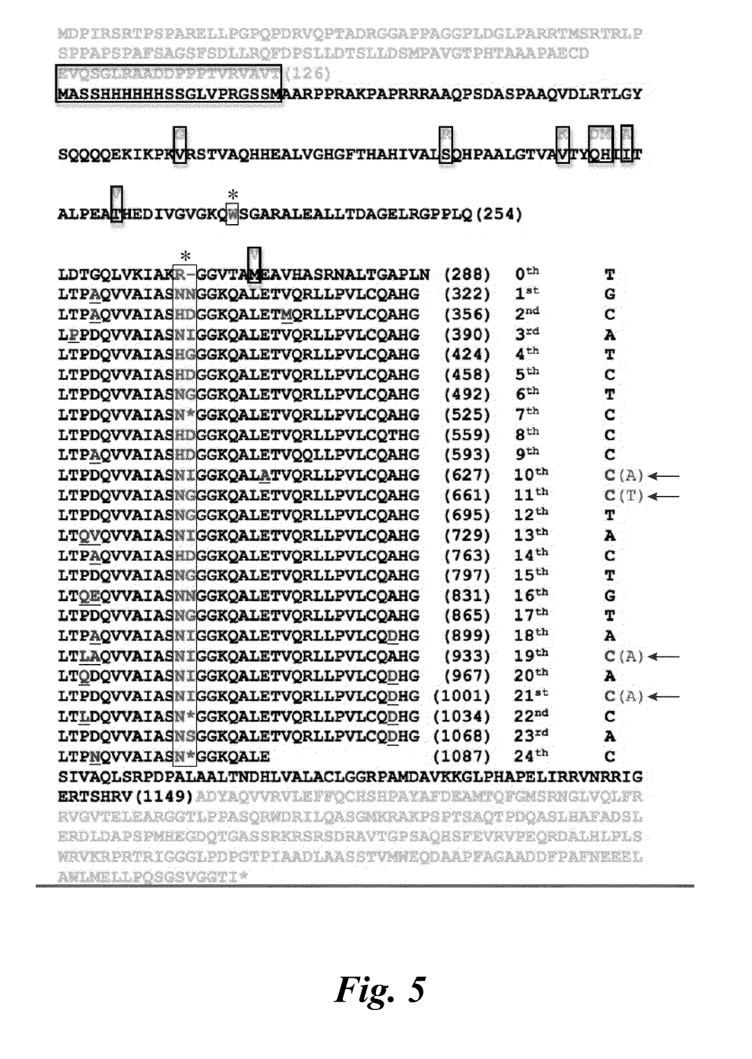

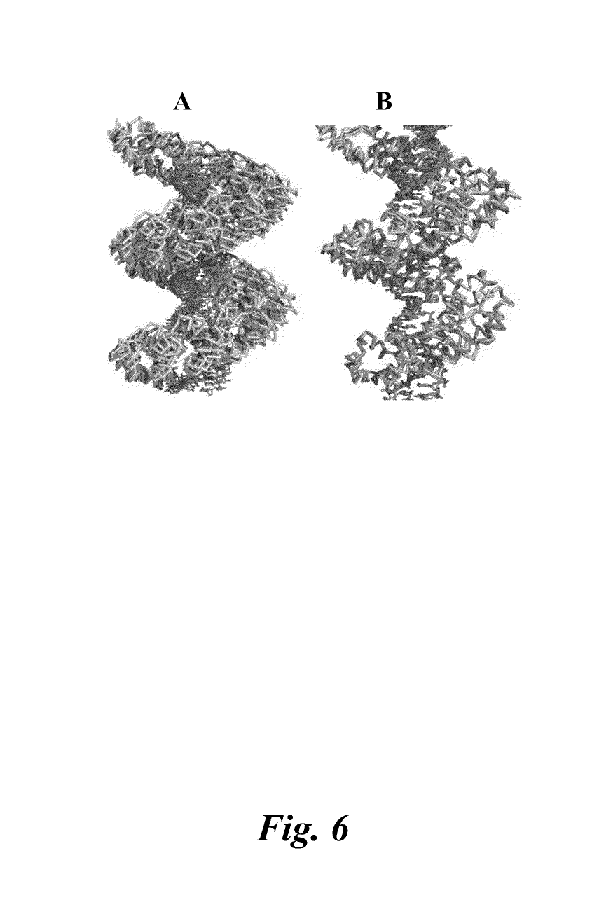

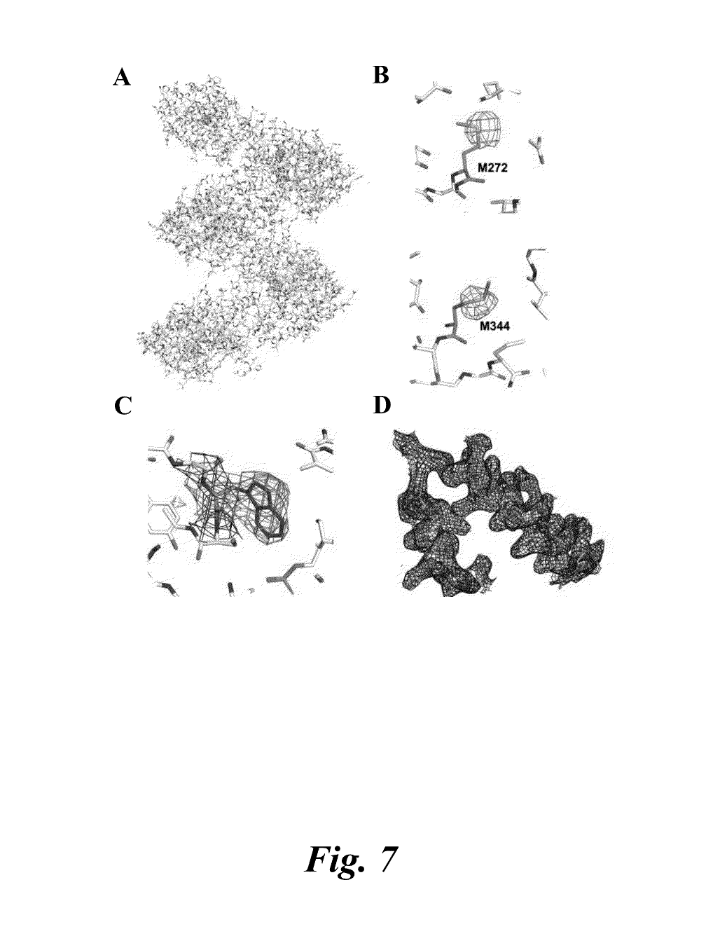

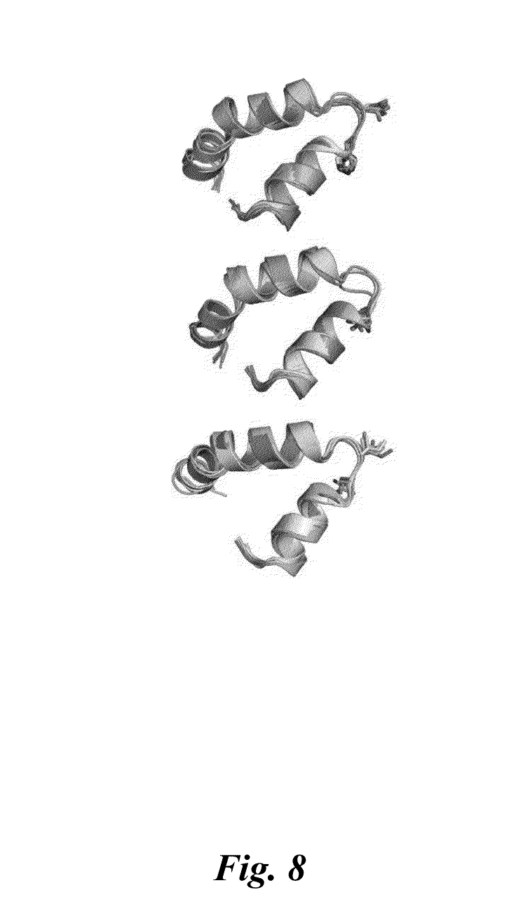

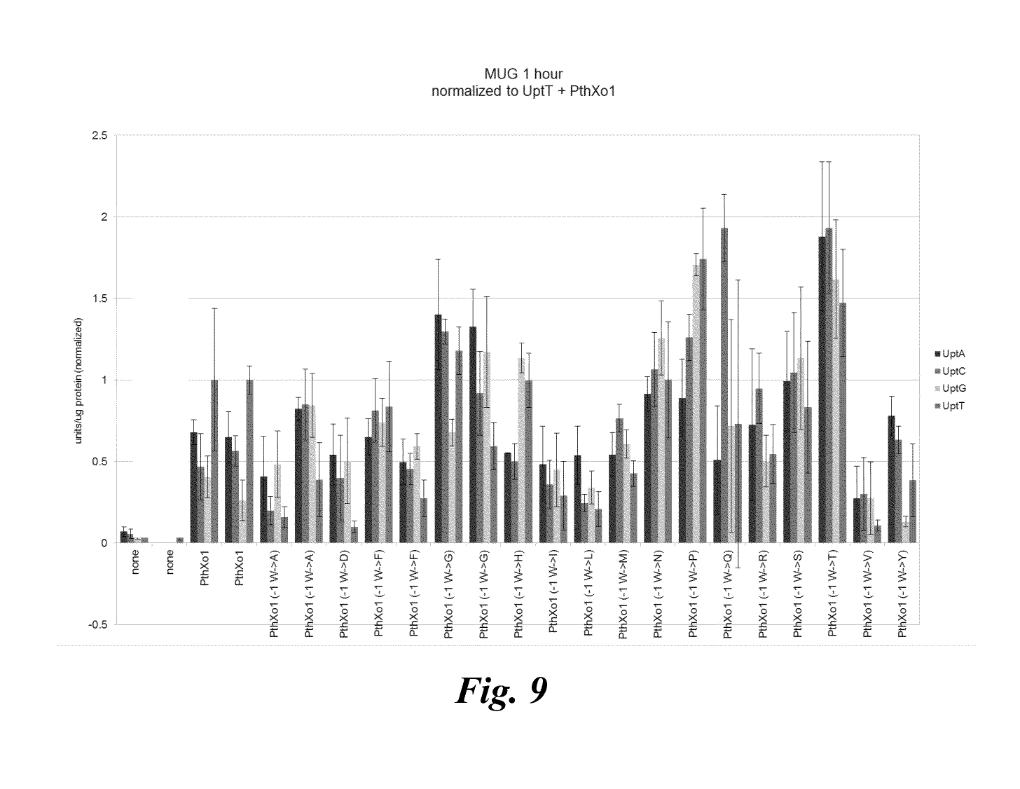

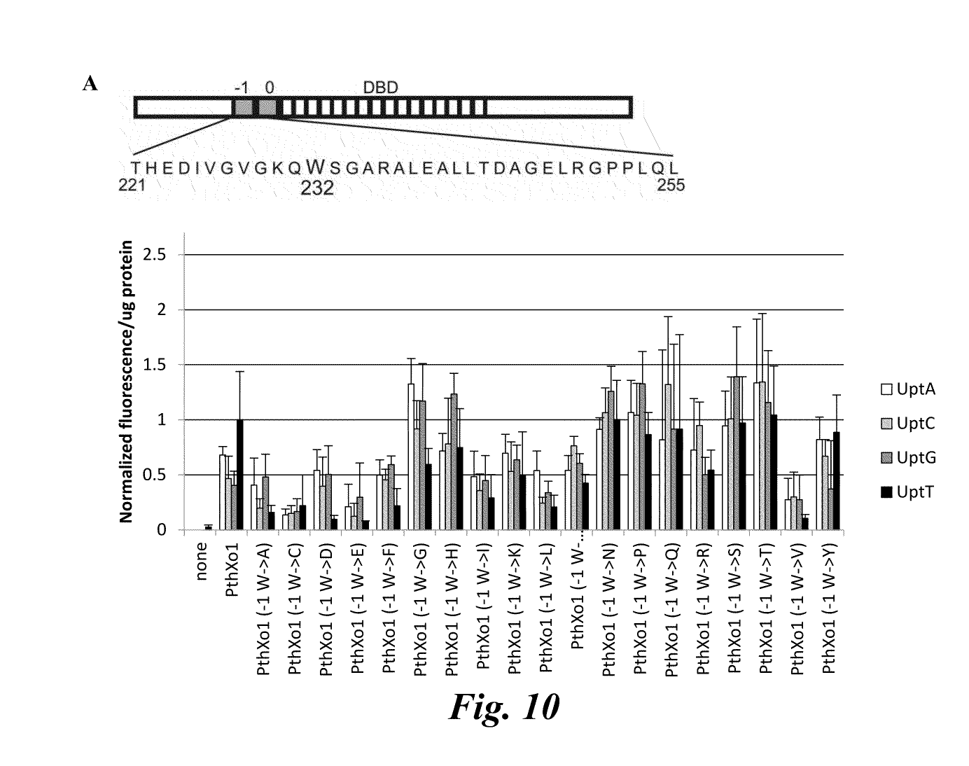

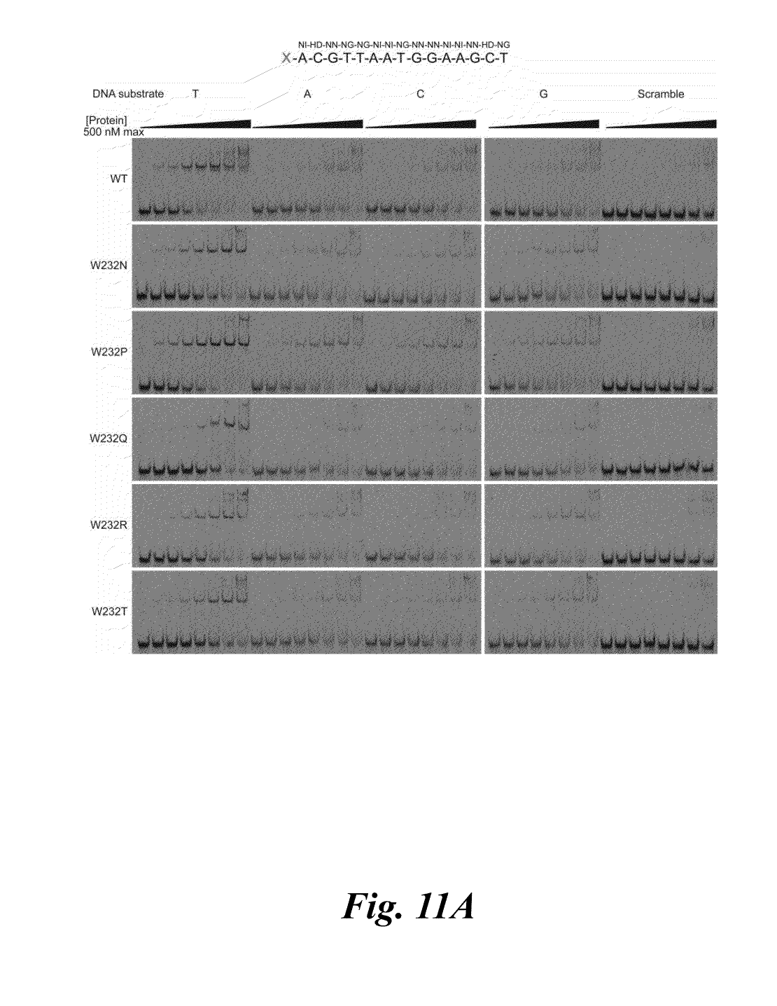

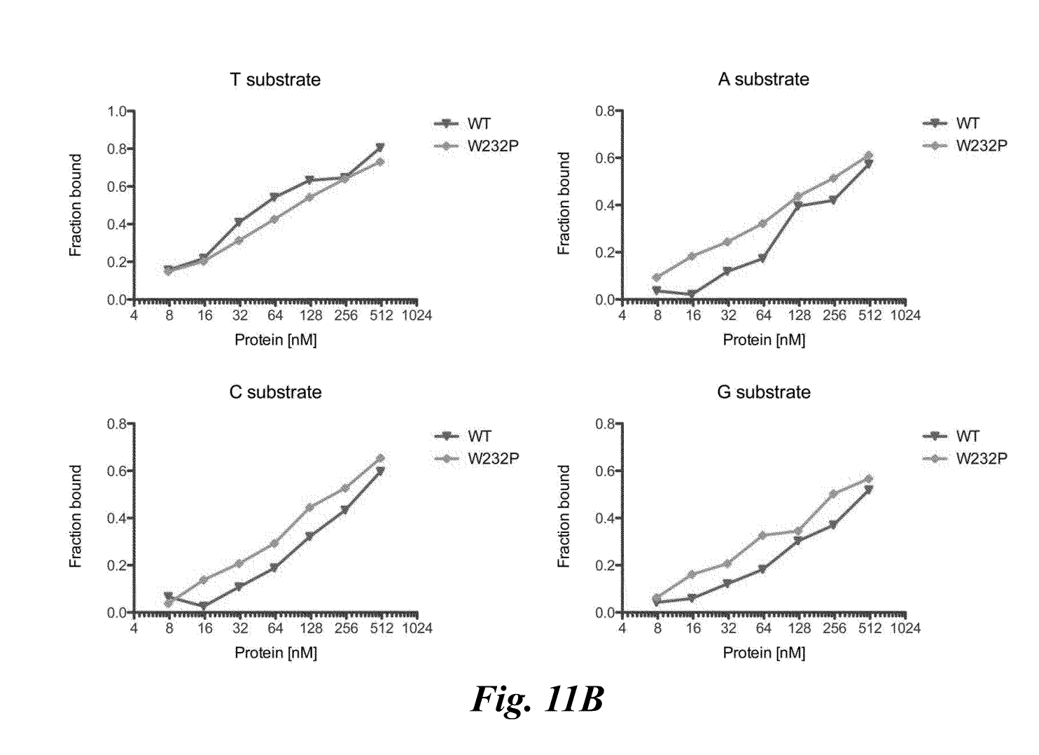

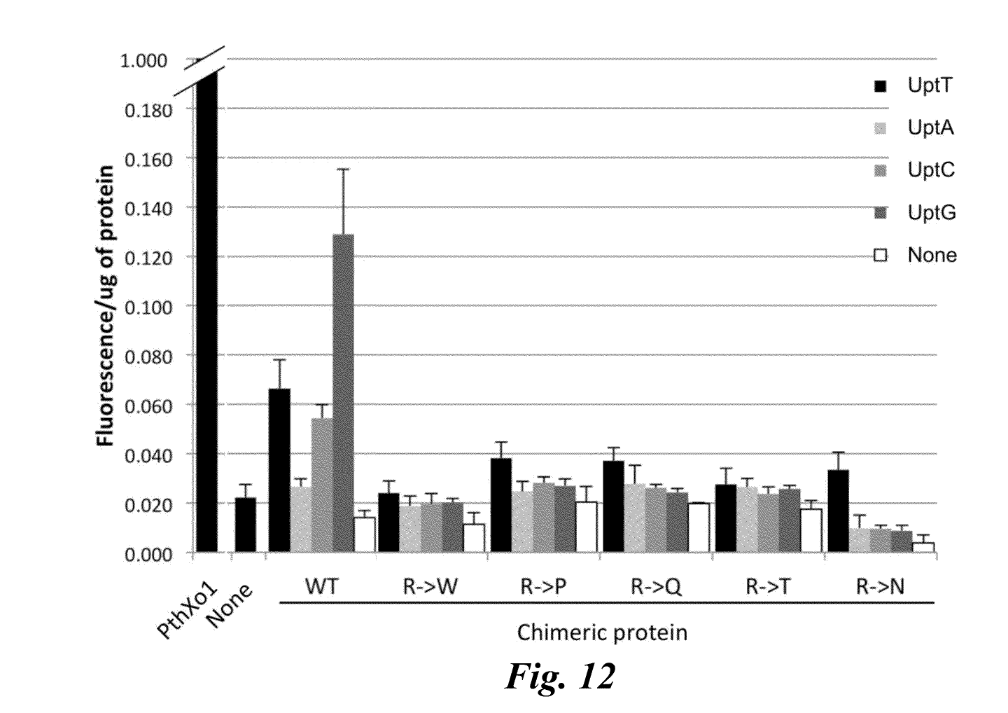

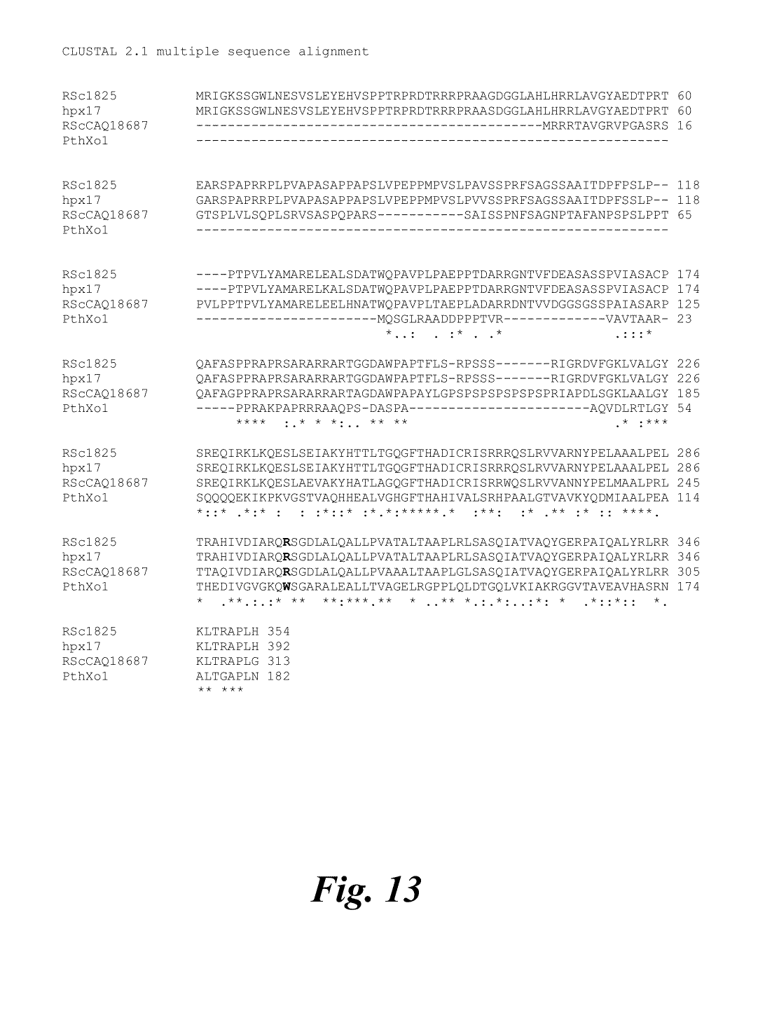

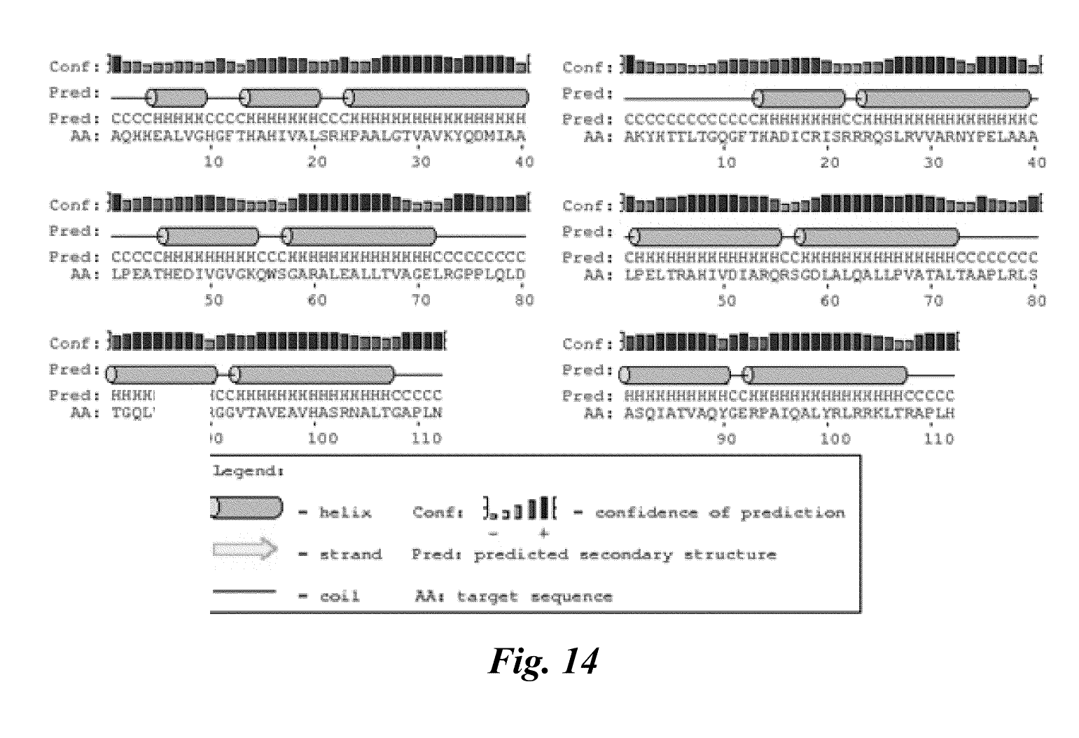

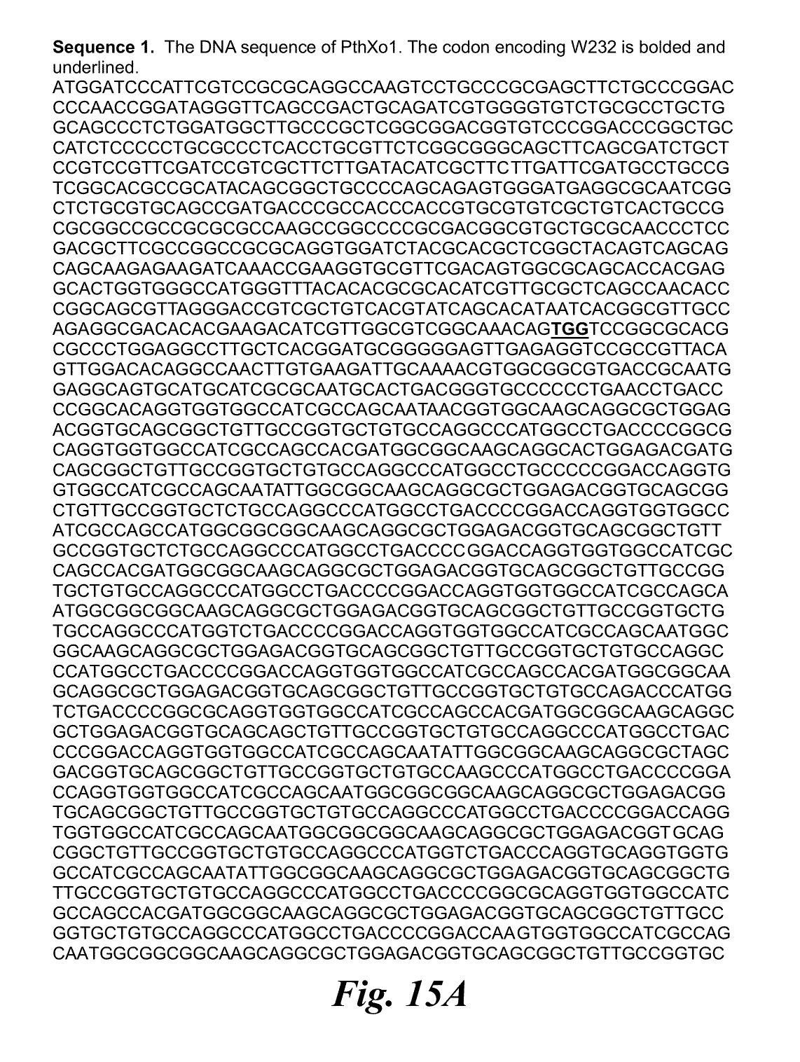

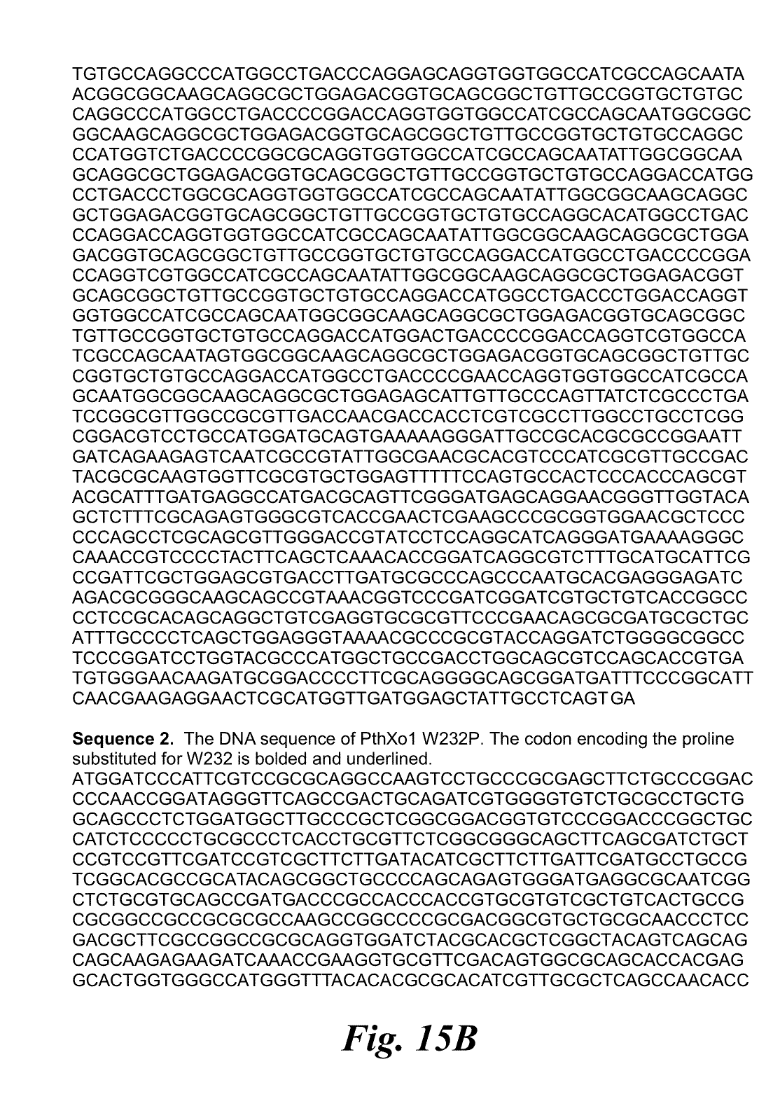

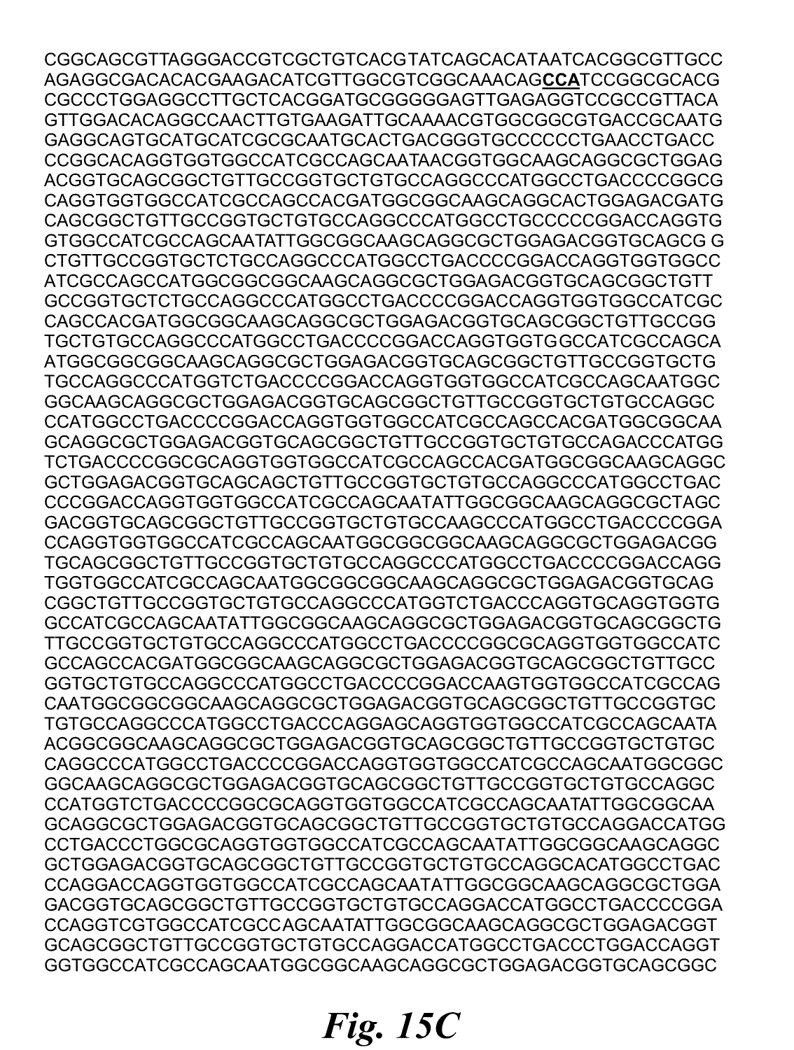

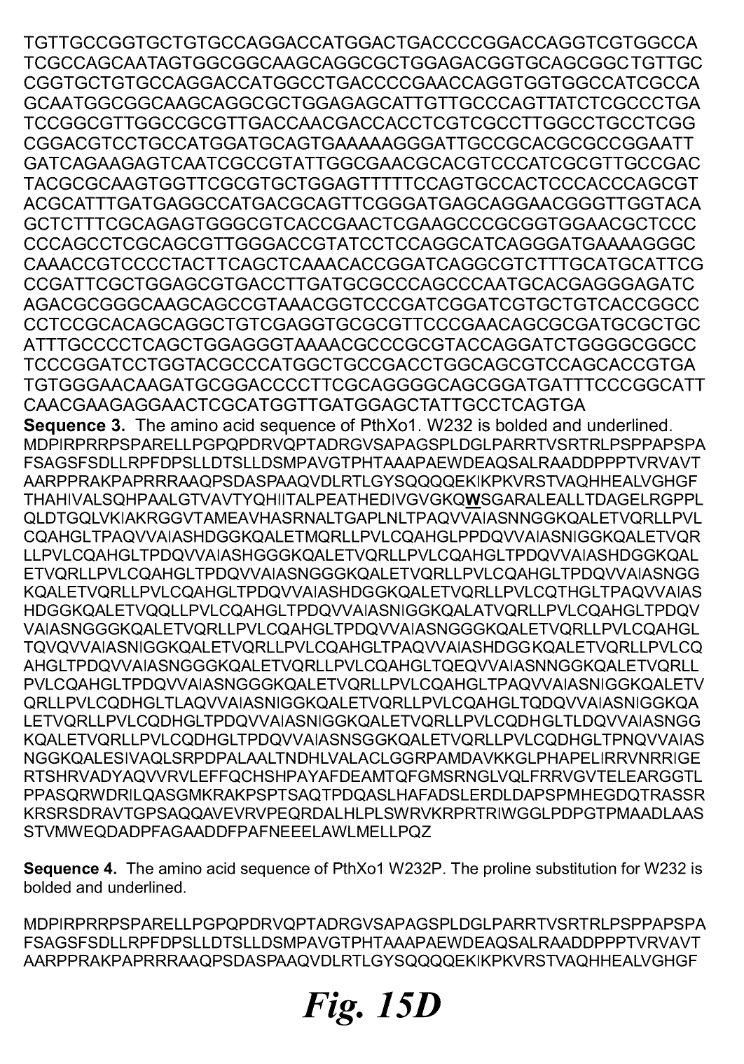

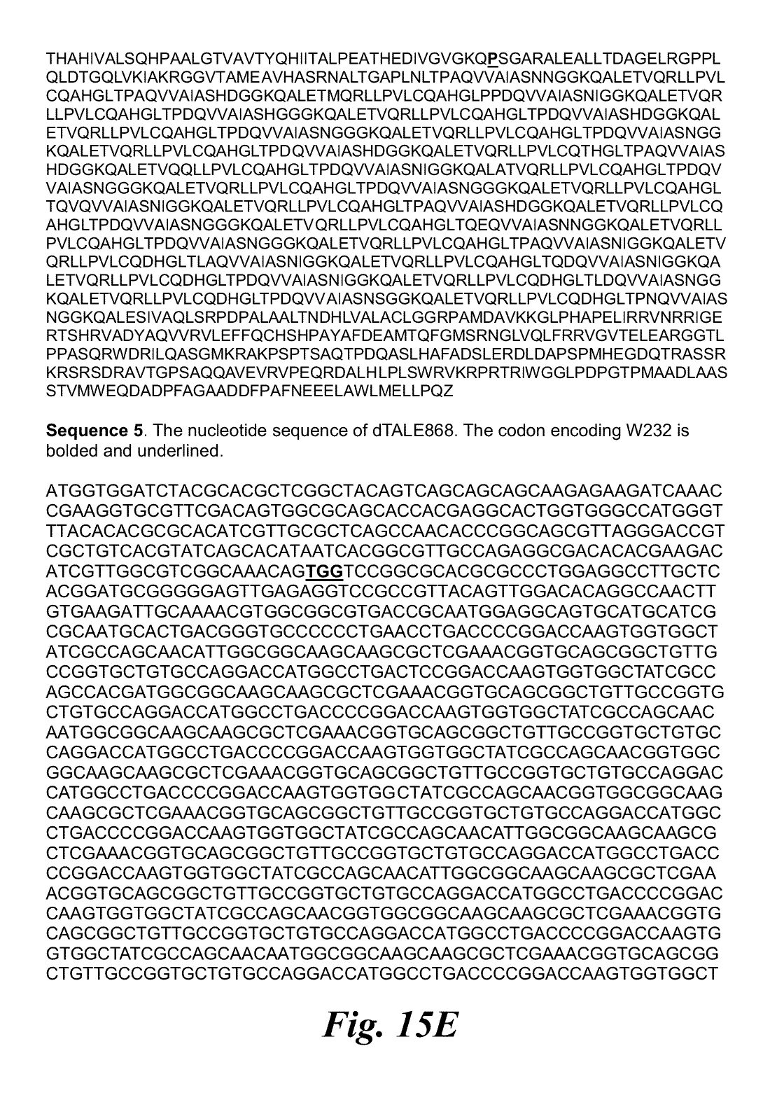

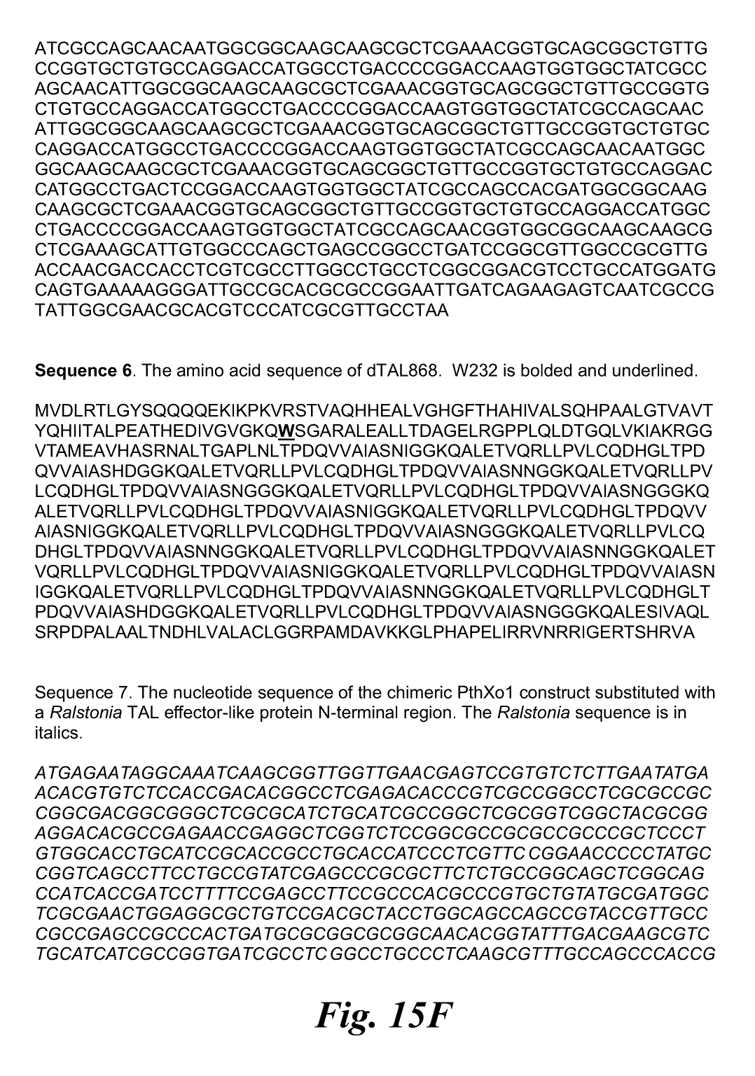

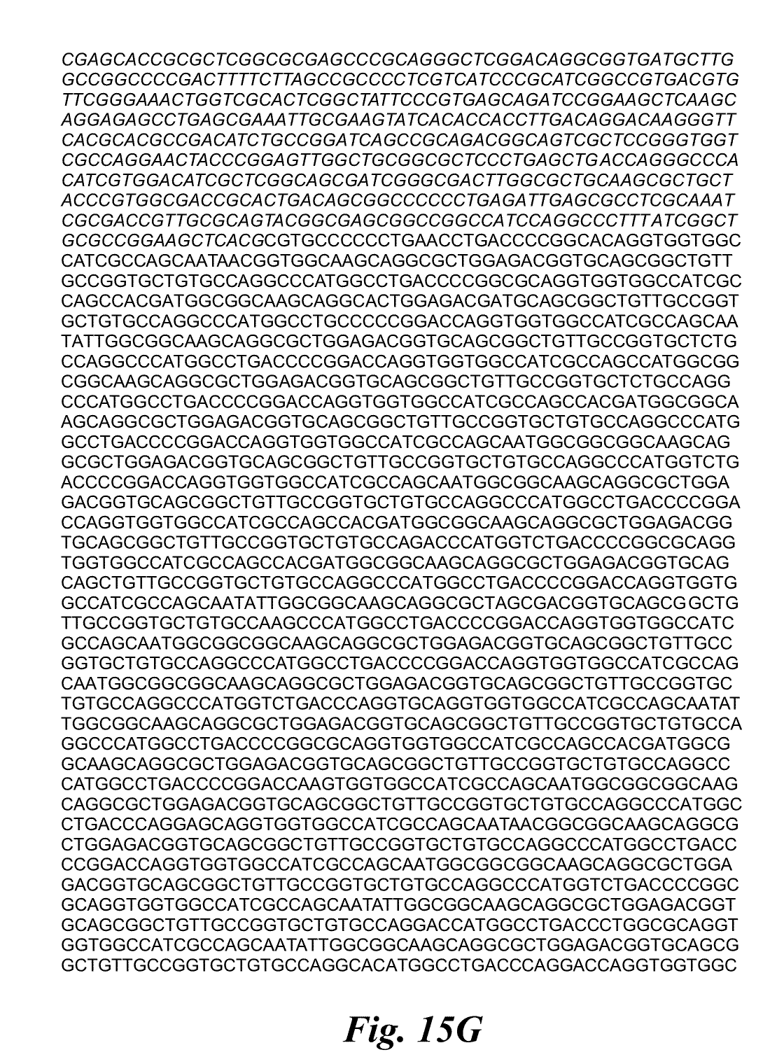

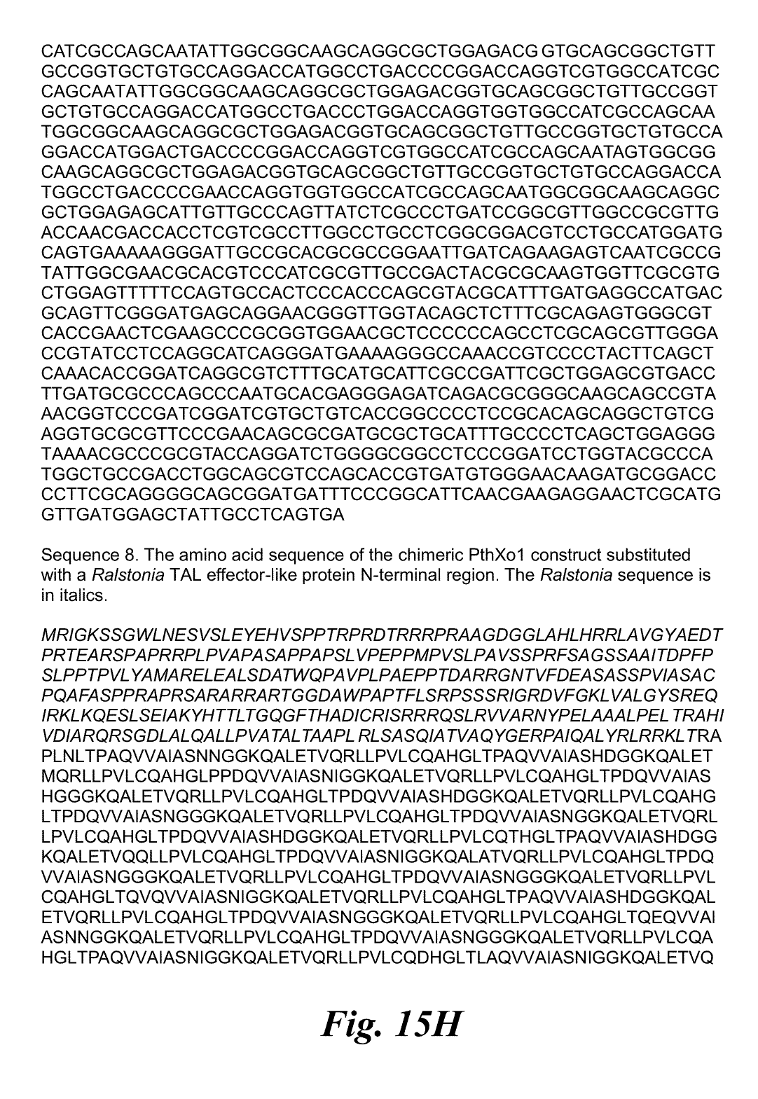

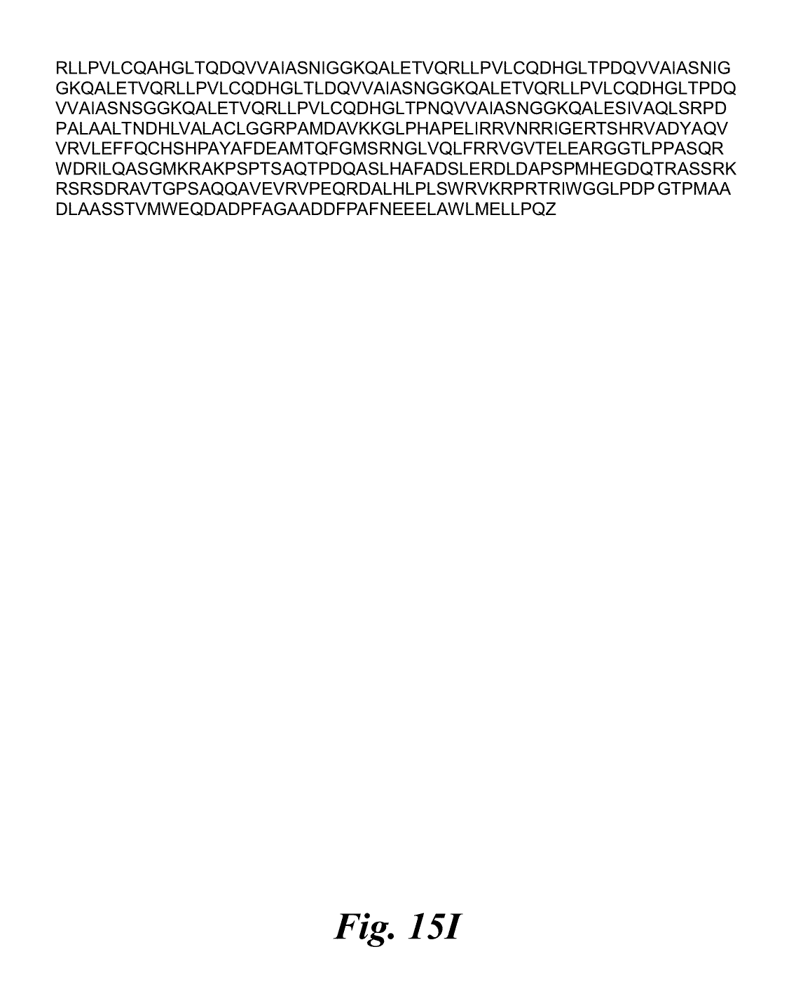

For structure determination, a protein construct corresponding to residues 127 to 1149 of the PthXo1 TAL effector (FIG. 1 and FIG. 5) was crystallized bound to a 36 base pair DNA duplex containing the effector binding site and flanking sequences ending in short 3′ overhangs. As described in Methods, the structure was determined using a novel high throughput computational approach in which structural models were iteratively used in molecular replacement runs. The best model was subsequently validated using a variety of features of electron density, such as anomalous difference peaks derived independently from selenomethionine-containing crystals and unbiased model-free features of density (FIG. 6). The final structure was refined to 3.0 Å resolution to values for R_{work}/R_{free} of 0.24/0.28 and displays excellent geometry.

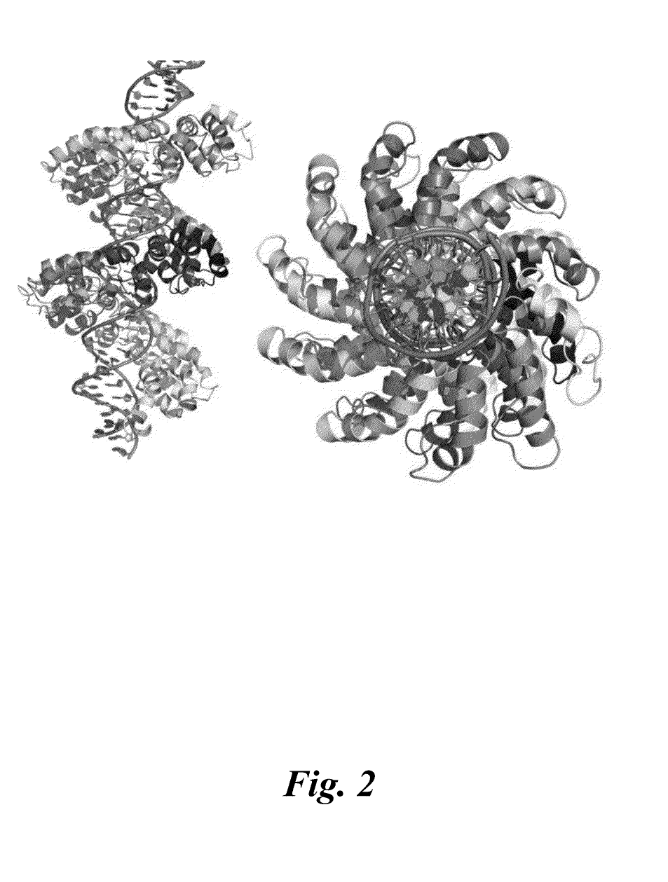

The structure consists of a long, relatively unperturbed B-form DNA duplex, within which the 25 consecutive bases of the DNA target site are intimately engaged in the major groove by a right-handed superhelical arrangement of TAL effector repeats (FIG. 2). The overall dimensions of the protein-DNA complex are approximately 60 Å×60 Å×90 Å. The quality of the electron density is excellent from repeat 1 through the end of repeat 21, and then becomes less well defined, appearing to indicate that the protein-DNA contacts at the extreme 3′ end of the target site are less well ordered. Crystallographic packing was facilitated by base pairing of the overhangs of the DNA duplex.

Each 34 amino acid repeat and the two 33 amino acid repeats (7 and 22) in the DNA-bound PthXo1 structure displays a highly similar fold corresponding to a two-helix bundle (FIG. 1). The helices span positions 3 to 11 and 14 to 33, locating the RVD in a tight loop between them. The second helix in each repeat is consistently kinked near a proline located at position 27. The sequential packing of consecutive helices within and between individual repeats is left-handed, in contrast to the right-handed packing of helices found in TPR proteins. Sequence-specific contacts to the target site are made exclusively by the second residue (position 13) in each RVD, to corresponding DNA bases located on the sense strand of the effector binding site. In contrast, the His or Asn side chains found at the first residue of the RVDs (position 12) contact the backbone carbonyl oxygen of position 8 in each repeat, thus forming a structural interaction that appears to constrain the conformation and position of the RVD-containing loop. In addition to the base-specific contacts made by the residues at position 13 in each RVD, additional nonspecific contacts are made to the DNA backbone by a lysine and glutamine found at positions 16 and 17. The average root-mean-square-deviation (rmsd) between any two individual repeats in the PthXo1 structure is approximately 0.8 Å for all atoms. With the exception of the 33 amino acid repeats, which have a shortened RVD loop discussed in detail below, the largest structural divergence between repeats generally corresponds to the RVD itself and in particular to the DNA-contacting 13th residue.

Binding of TAL effectors to DNA has been proposed to involve large-scale conformational changes and compaction of the protein in order to wind around the target site. This hypothesis is consistent with the contacts observed within the core of each repeat as well as those that are observed in the interfaces between sequential repeats. The core of each repeat is largely composed of small hydrophobic residues, located at positions 1, 6 and 8 in the first helix and positions 19, 22 and 26 in the second helix. In contrast, the interface between the individual repeats contains a more diverse set of both hydrophobic and hydrophilic residues and includes several repeating sets of partnered side-chains. For example, the glutamine side chain of position 5 in a given repeat is usually positioned within contact distance to the aspartate side chain of residue 4 in the next repeat. Similarly, glutamine 17 of most repeats contacts lysine 16 of its following neighbor, and residues 21 and 20 of sequential repeats form a threonine-glutamate pair.

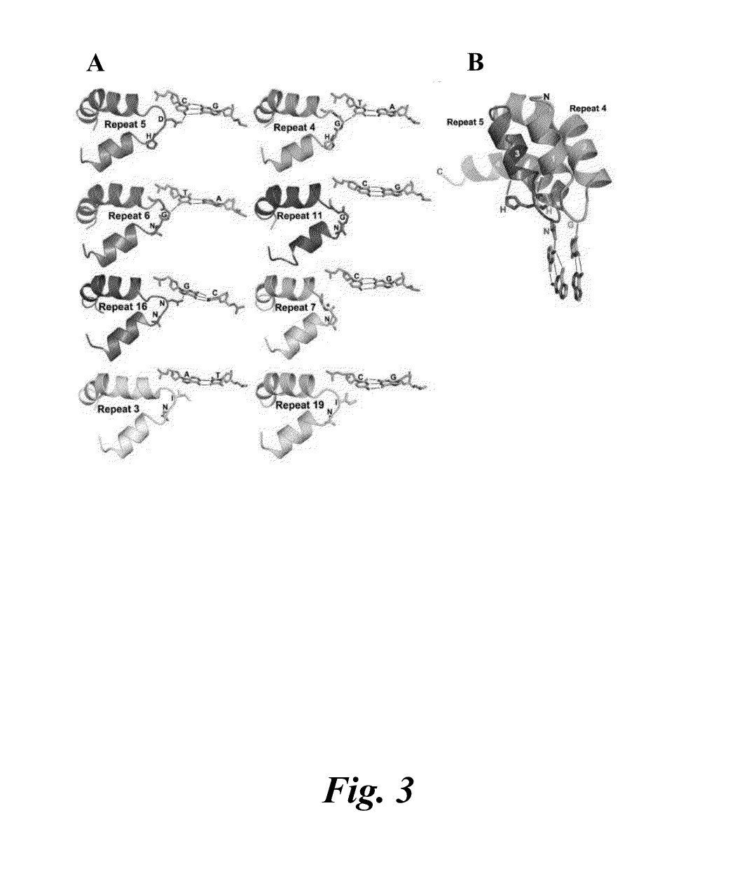

The PthXo1-DNA structure displays five HD-containing repeats all aligned to cytosines, four ‘NG’ repeats and one ‘HG’ repeat aligned to thymines and one more ‘NG’ repeat aligned to cytosine, seven ‘NI’ repeats aligned with four adenosines and three cytosines, two ‘NN’ repeats both opposite a guanosine, and two ‘N’ repeats paired with cytosines. The contacts observed in the structure (FIG. 3) correlate well with the specificity and fidelity of individual repeats (or lack thereof) that has been described via bioinformatic and genetic analyses, particularly for HD, NG, HG, NN and N*, and to a lesser extent for NI. The sole NS in PthXo1 and an additional N* are in the last full repeat and the half repeat respectively, which are disordered in the structure.

In the ‘HD’ RVDs, the aspartate residue makes van der Waals contacts with the edge of the corresponding cytosine base and a hydrogen bond to the cytosine N4 atom. Contacts between cytosine bases in protein-DNA complexes and charged acidic side chains, which exclude alternative base identities via physical and electrostatic clash have been observed previously in a wide variety of solved sequence-specific protein-DNA complexes.

In contrast, both the ‘NG’ and ‘HG’ containing repeats make a contact in which the backbone alpha carbon of the glycine residue forms a nonpolar van der Waals interaction with the methyl group of the opposing thymine base (average distance ˜3.3 Å). At the one position where an ‘NG’ RVD is aligned opposite a ‘less preferred,’ cytosine base, the backbone carbonyl and alpha-carbon of the same glycine residue displays a less favorable, far more distant contact (˜6 Å).

The second asparagine residue in the ‘NN’ RVDs is positioned to make a hydrogen bond with the N7 nitrogen of an opposing guanine base. Unlike those described above, this RVD appears to have roughly equal affinity for either of two bases, guanosine or adenine, based on association frequencies and TAL effector activity and binding assays on alternative targets. The availability of an N7 nitrogen in either purine ring appears to explain that observation.

PthXo1 contains two copies of the 33 residue repeat ‘N*’ (repeats 7 and 22). In the consensus repeat sequence, RVDs are followed by the sequence GGK. So although the ‘N*’ repeat was so designated to distinguish it from the ‘NG’ type, it is equivalent to an ‘NG’ repeat with one of the three neighboring glycine residues (positions 13, 14, and 15) missing. The crystal structure indicates that this deletion results in a truncated RVD loop that extends less deeply into the DNA major groove, with the glycine at position 13 located a considerable distance (over 6 Å) from the corresponding base on the sense strand (in contrast to the glycine at position 13 in the ‘NG’ repeat discussed above). Consistent with this observation, the ‘N*’ RVD displays relatively lacks apparent specificity, associating with cytosine or thymine with roughly equal frequency, and to some extent with adenosine and guanine as well. The presence of this RVD at repeat 22 may reduce the overall affinity of interaction at that end of the PthXo1-DNA complex, contributing to the apparent disorder in that region.

Finally, NI, which is the second most common RVD overall, accounting for roughly 20% of all TAL effector repeats, occurs seven times in PthXo1. Despite its ubiquitous distribution in TAL effectors, it displays an unusual contact pattern to adenosine or cytosine bases. The aliphatic side chain of the isoleucine residue is observed to make non-polar van der Waals contacts to C8 (and N7) of the adenine purine ring, or to C5 of the cytosine pyrimidine ring. These contacts would appear to necessitate desolvation of at least one polar atom in the adenosine ring, without the formation of a compensating hydrogen bond to the protein. This contact therefore might reasonably be expected to represent a reduced affinity interaction. The reason for the apparently strong overall preferential association of this RVD with adenosine in the majority of TAL effector-target alignments therefore is not immediately clear from the structure. The presence of four consecutive ‘NI’ RVDs in the C-terminal end of the PthXo1 structure (at repeats 18 through 21) may also contribute to the relative disorder apparent at that end.

In addition to illustrating the structural basis for several RVD-nucleotide associations, the PthXo1 structure reveals, N-terminal to the canonical repeats, not one but two degenerate repeat folds that appear to cooperate to specify the thymine that is consistently found immediately preceding the RVD-specified sequence of TAL effector targets. Residues 221 to 288 form two partially folded regions that loosely recapitulate the topology of their neighboring repeats. We have designated these as the 0th and −1st repeats. Residues 221 to 239 and residues 256 to 273 each form a helix and an adjoining loop that resembles helix 1 and the RVD loop in the canonical repeats; the remaining residues in each region are disordered. The ordered portions of those two N-terminal regions converge near the 5′ thymine base, with the indole ring of tryptophan 212 (in the −1st repeat) making a van der Waals contact with the methyl group of that base. That tryptophan residue, as well as the surrounding residues, are highly conserved across available intact TAL effector sequences, providing an explanation for the ubiquity of the thymine in TAL effector binding sites. Interestingly, some custom TAL effectors designed to target sequences that happened to be preceded by a cytosine rather than a thymine did so efficiently. Though less favorable, the packing of tryptophan 212 would be expected to accommodate this substitution, but not substitutions of a purine, which so far have not been observed in functional targets.

The protein-DNA complex studied leaves some questions unanswered, such as the structure of the N and C-terminal portions of TAL effectors required for translocation and interaction with host transcriptional machinery. And, because of the apparent disorder at either end, it does not yet precisely define the minimal TAL effector DNA binding domain. At the same time, the structure provides insight into some questions that it does not answer directly, for example, the nature of the 35 amino acid repeat sometimes found in functional TAL effectors. Sequence alignments (not shown) of the 35 residue consensus with that of the 34 residue repeats from the structure indicates that the additional residue (a proline) at position 33 would be found within a relatively disordered turn region that connects the helices of one repeat to the next. It can be predicted then that the 35 and 34 residue repeats are structurally similar and functionally indistinguishable. Likewise, although the sole ‘NS’ RVD is in an apparently disordered part of the PthXo1-DNA complex, the overall homogeneity of the repeat structures and the consistent role of the first RVD residue in stabilizing the RVD loop to facilitate contacts of the second RVD residue with the DNA base should make it possible to computationally model the potential nucleotide interactions of NS, as well as those of rare or novel RVDs.

### Methods

### Cloning of the PthXo1 Construct

Plasmid pEH1-pthXo1 carrying the pthXo1 gene from Xanthomonas oryzae pv. oryzae strain PXO99A was digested with SphI and the 2898 bp fragment containing the repeat region was purified and cloned into pCS466 cut with the same enzyme, producing pAH103. pCS466 is a derivative of Gateway® entry vector pCR8/GW/TOPO (Invitrogen) that carries the tal1c gene from Xanthomonas oryzae pv oryzicola missing the SphI fragment that comprises its repeat region. For protein expression, plasmid pET15-HE was digested with NcoI and NotI and ligated with an adaptor to introduce a new NotI restriction site that would allow introduction of the NotI/BsrG1 fragment of pthXo1 from pAH103 in-frame with the N-terminal 6-histidine affinity tag. That subcloning step resulted in removal of the first 126 codons of the TAL effector gene. Then, coding sequence for 224 amino acids from the PthXo1 C-terminus was removed by digestion with BmgBI and BsrGI and ligation to an adaptor to introduce a stop codon at position 63 relative to the end of the repeat region, corresponding to position 1150 of the full length PthXo1 protein, yielding the final expression vector pAC-EXP31c. Plasmids were maintained in E. coli XL10 gold cells (Invitrogen).

### Expression and Purification of the PthXo1 Construct

The PthXo1 construct (FIG. 5) in pAC-EXP31c was expressed in E. coli strain BL21 (DE3) pLysS (RIL). After transformation and growth on LB plates supplemented with ampicillin, approximately colonies were simultaneously harvested and used to inoculate 100 mL of LB broth augmented with 1 mM magnesium sulfate and ampicillin. After two hours of shaking at 37° C., the entire starter culture was then used to inoculate one liter of the same culture media, which was grown to an OD600 of approximately 1.2 to 1.4. IPTG was added to a final concentration of 0.4 mM and the cultures were shaken overnight at 16° C. Cells were harvested by centrifugation and resuspended in 100 mL lysis buffer (0.1 M Tris-Cl pH 7.5, 0.2 M NaCl, 10% glycerol and 0.1 g of T4 lysozyme). The cells were lysed by sonication, and after a second round of centrifugation the lysate was clarified by filtration and loaded directly onto a heparin affinity column. After washing with several column volumes of buffer A (identical to the lysis buffer but without lysozyme), a 100 mL salt gradient (ranging from 0.2 M NaCl to 1.0 M NaCl in the same buffer) was applied at a flow rate of 3 mL per minute, resulting in elution of the PthXo1 construct at approximately 0.5 M NaCl. The protein was diluted with four volumes of buffer A (reducing the NaCl concentration to approximately 0.22 M NaCl) and then applied to a Hi Trap SP-XL cation exchange column (GE BioSciences). The PthXo1 construct was observed to flow directly through the second column, resulting in significant further purification (to approximately 95% of total visible protein as estimated by SDS-Page analysis). The protein was concentrated to approximately 3 mg/mL (24 micromolar) concentration using a Centricon filter apparatus with a 50,000 dalton molecular weight cut-off and then used immediately in crystallization experiments.

### Crystallization and X-Ray Data Collection

The purified PthXo1 construct described above was screened for crystal growth in the presence of a panel of DNA constructs, all containing the previously described wild-type 25 base pair binding site for the TAL effector. The DNA constructs used in the screening experiments were designed to sample a range of duplex lengths (ranging from 32 to 41 base pairs) and a variety of unique structures at both ends (including 5′ or 3′ overhangs of various lengths and blunt ends) (Table 2). The DNA was present in excess relative to protein (usually at molar ratios of 1.5 DNA to 1.0 protein) in these experiments. All initial screening was performed using several commercially available sparse matrix crystallization kits with a TTP LabTech ‘Mosquito’ crystallization robot, where 50 nanoliter volume sitting protein drops were mixed with an equivalent volume of reservoir solution and then equilibrated over 100 microliter reservoirs. All screening was performed at room temperature and any promising hits were then scaled up into hanging drop geometries under the same conditions, with 1.5 microliter hanging protein drops (plus an equal volume of reservoir contents) equilibrated against 500 microliter reservoirs.

The highest quality crystals were grown in the presence of DNA constructs containing a 38 basepair duplex (that included the wild-type TAL effector recognition site) flanked by mutually cohesive, 2-base 3′ overhangs. The sequences of the crystallization oligonucleotides were (the bases corresponding to the effector recognition site are indicated by bold underlined font):

(SEQ ID NO: 3)
5′-TAGATATGCATCTCCCCCTACTGTACACCACCAAAAGT-3′
(top strand)
(SEQ ID NO: 4)
3′-CAATCTATACGTAGAGGGGGATGACATGTGGTGGTTTT 5′.
(complementary bottom strand)

The reservoir condition for these crystals contained 0.1 M MES pH 6, 0.25 M sodium acetate and 14% PEG400. The crystals grew in space group P212121 (average dimensions a=95.6 Å, b=248.5 Å, c=54.6 Å) and contain approximately 40% solvent (corresponding to a calculated value for Matthew's specific volume (VM) of approximately 2.5 Å3/dalton).

The crystals were transferred to artificial mother liquor containing elevated concentrations of PEG400 (typically 30% v/v), harvested individually in fiber loops and cryoprotected by immersion into liquid nitrogen. The crystals generally diffracted to approximately 4.5 Å resolution on a home X-ray source (a Rigaku HF-007 rotating anode generator and an RAXIS-IV++ phosphor imaging plate area detector) and to as high as 3 Å resolution at a synchrotron X-ray source. During the course of crystallization and phasing work, we observed that the addition of 10 mM potassium perrhenate often improved diffraction characteristics of the crystals. The estimated crystal mosaicity was typically 0.7 to 1.0°.

X-ray data (Table 1) used for the structure determination collected at the Advanced Light Source (ALS) synchrotron facility (Berkeley, Calif.) at beamline 5.0.2 using a CCD area detector. An additional low resolution SeMet dataset that was used as a valuable source of model and phase validation was collected at the Advanced Photon Source (APS) at beamline 21-ID-F. Typical data collection was performed at wavelengths ranging from 0.91 to 1.37 Å (usually corresponding to X-ray absorption edges for various heavy atom scatterers that were incorporated into the protein or DNA, or that were added via heavy-atom soaks and cocrystallization experiments). Data from the ALS was collected on an ADSC CCD area detector with a crystal-to-detector distance of 350 mm and exposure times of 1 second per 0.5 degree crystal rotation. For all data sets, a full 360° of exposures was collected to maximize data redundancy. All data was processed using the HKL2000 software package.

TABLE 1
Crystallographic data and refinement statistics

| | | |
|---|---|---|
| Dataset | WT | SeMet |

DATA STATISTICS

| X-ray source | ALS 5.0.2 | | APS 21-ID-F | |
|---|---|---|---|---|
| Wavelength (Å) | 1.177 | | 1.378 | |
| Space group | P2₁2₁2₁ | | P2₁2₁2₁ | |
| Unit Cell (Å) | a = 95.6 | | a = 100.7 | |
| | b = 248.5 | | b = 247.8 | |
| | c = 54.6 | | c = 54.2 | |
| Resolution (Å)^{a} | 50-3.0 | (3.11-3.0) | 50-4.0 | (4.14-4.0) |
| R_{merge} (%) | 0.121 | (0.431) | 0.087 | (0.139) |
| I/σ (I) | 9.3 | (3.5) | 10.3 | (3.7) |
| Redundancy | 5.6 | (4.9) | 4.9 | (5.0) |
| Completeness (%) | 96.6 | (90.4) | 95.8 | (97.7) |

| Mosaicity (°) | 0.8 | 0.8 |
|---|---|---|
| Unique Reflections | 25841 | 11591 |
| REFINEMENT | | |
| R_{work} | 0.264 | |
| R_{free} | 0.294 | |
| Protein Atoms | 6086 | |
| DNA Atoms | 1552 | |
| Heteroatoms (waters) | 216 | |
| Rmsd bond lengths (Å) | 0.021 | |
| Rmsd bond angles (°) | 2.4 | |
| Average B factor (Å²) | 85.1 | |
| Ramachandran (% core, | 73.6%, 26.4%, | |
| allowed, generous, | 0%, 0% | |
| disallowed) | | |

### Computational Fold and Structure Prediction, Modeling and High-Throughput Molecular Replacement

Overview:

To generate starting models for crystallographic refinement, we developed a hybrid molecular modeling strategy that combined de novo structure prediction together with iterative structure refinement guided by Phaser molecular replacement (MR) searches. De novo modeling was first used to construct a large pool of candidate models. A global Phaser MR search was then performed for each model, allowing either of the two alternative space groups (P212121 and P21212) compatible with the initial experimental data. The top-scoring models in this first round of MR searches were iteratively refined by alternating between a fragment-based conformational resampling protocol (implemented in the Rosetta software package) and local-refinement Phaser MR searches. During the de novo modeling and in the early stages of refinement, perfect structural symmetry was enforced across all 23 repeats and in the conformation of the DNA duplex (i.e., the base-step transforms between successive base pairs were identical throughout the duplex). This symmetry constraint was relaxed in the later stages of refinement.

De Novo Modeling:

We developed a symmetrical structure prediction protocol tailored to the TAL system, which was used to assess the steric feasibility of a 1-1 mapping between TAL repeats and B-form DNA basepairs and to generate an initial de novo model of TAL-DNA interactions for several RVD types. In this approach, the two key constraints of structural symmetry and RVD-DNA contact are used to focus sampling on regions of conformational space that are compatible with the current model of TAL effector function. Fully symmetrical protein-DNA models are constructed by anchoring each successive 34 amino acid repeat to its cognate base pair by a rigid-body linkage whose geometry is sampled from a library of amino-acid-base contacts seen in protein-DNA complexes. The protein anchor point for this linkage is randomly selected from the two possible RVD positions (repeat residues 12 and 13) at the start of each independent simulation, and the amino acid identity of the RVD-nucleic acid pair being modeled (for example, NI-A:T or HD-C:G) then determines the contact library used for sampling that linkage. Symmetry of these rigid-body protein-DNA linkages is maintained throughout the simulation by making identical updates to all linkages during the Monte Carlo moves that sample the protein-DNA docking mode. The backbone conformation of the repeat units is simultaneously sampled by means of symmetrical fragment-replacement moves in which a set of consecutive backbone torsion angles taken from a protein of known structure and compatible local sequence is inserted in symmetrically related windows throughout the current model (for example, a 3 residue fragment might be inserted at residues 15-17, 49-51, 83-85, etc). To ensure symmetry of structural context for each protein repeat, simulations were conducted with symmetrical DNA duplexes in which the base-step geometry was identical throughout, ensuring that interface contacts between successive repeats would be identical as well (since symmetry of the protein-DNA linkages is enforced).

Iterative Model Refinement:

The de novo modeling protocol was used to generate a diverse pool of 20,000 fully symmetric, 23-repeat models (4,000 models for each of the RVD-nucleic-acid pairs HD-C, NI-A, NG-T, NN-G, NN-A). In each model, the same RVD-nucleic acid pair was present in each repeat, allowing the enforcement of perfect conformational symmetry during de novo sampling. The sidechains of these models were trimmed back to the C-beta atom, the RVD-containing loops (repeat residues 11-15) were deleted, and global Phaser MR searches (Phaser keyword mode “MR_AUTO”) were performed for all of the models. The Phaser scores for this initial round of de novo models were encouraging (LLG scores in the range of 50-110; TFZ scores reaching 10.0), however there was considerable diversity in the conformations and unit-cell placements of the top models (FIG. 6, panel A). The 60 top scoring models from this initial pool were then refined by multiple rounds of fragment-based rebuilding of the protein backbone conformation, resampling of the protein-DNA binding mode, and perturbation of the DNA internal conformation followed by local MR refinement searches (Phaser keyword mode “MR_RNP”). In the first few rounds, total symmetry of the protein repeats and the DNA internal conformation was enforced, thereby reducing the space of possible conformations and enhancing sampling efficiency. Once this process appeared to converge, the symmetry constraint was relaxed and the amino acid sequence of the 23 complete repeats of Pthxo1 was threaded onto the models, with the RVD loops in repeats 7 and 22 rebuilt in order to accommodate the 1-residue ‘N*’ deletions. We began to include sidechains during MR searches, and MR searches were followed up by short crystallographic refinement runs with the Phenix structure refinement program. During symmetric refinement we had seen high-scoring models with several alternative placements in the unit cell, corresponding to shifts of 1-2 repeats in either direction. Once the N* repeat loops were built and the Pthxo1 sequence threaded onto the models, we began to see convergence on one unique repeat register in the unit cell, corresponding to a single placement of the N* loops within the otherwise highly symmetric repeat structure. Progress in these later rounds of fragment-based refinement was monitored by the “xray_target” score for the work reflections as reported by phenix.refine.

FIG. 6, panel B shows conformational diversity present in several representative models from later rounds of refinement; this level of diversity can be contrasted with that present in the initial population of de novo models (FIG. 6, panel A).

### Crystallographic Modeling, Phase Validation and Structural Refinement

Protein models (with DNA removed) from the computational fold prediction and molecular replacement runs described above were edited to remove DNA atoms and used in a second round of molecular replacement. The top scoring solutions were then subjected to a round of simulated annealing refinement and difference maps were calculated. The best MR solution yielded clear density for the bound DNA duplex, allowing both strands and the corresponding bases to be built. The resulting model was then placed into a separate round of MR using a data set collected from crystals grown with selenomethionyl-derivatized protein. Analysis of anomalous difference Fourier maps using that data produced peaks corresponding to the predicted positions of Met 271 (in the ‘0’ repeat) and Met 344 (in repeat 2) (FIG. 7, panels A and B).

The protein structure was built from the ‘inside out’, by systematically deleting each repeat region, performing rounds of simulated annealing refinement, then calculating unbiased omit maps and rebuilding the repeat region. During the process of building, a number of features of density provided additional validation of the structure (FIG. 7), including the presence of unambiguous density corresponding to the indole side chain of Trp 232 (the only tryptophan in the protein sequence) and very clear difference density corresponding to abbreviated RVD loops for the ‘N*’ containing repeats (7 and 22).

Final stages of refinement and model building were carried out using programs COOT and REFMAC. The geometric quality of the final model was validated using PROCHECK and WHATIF, as well as the validation server tools provided by the RCSB Protein Data Base.

TABLE 2
DNA constructs used during crystallization trials.

| Construct | Sequence |
|---|---|
| Pthxo1-1 (SEQ ID NO: 5) | GTTAGATATGCATCT CCCCC TACTGTACACCACCAAAAGT |
| | CAATCTATACGTAGAGGGGGATGACATGTGGTGGTTTTCA |
| Pthxo1-2 (SEQ ID NO: 6) | GTTAGATATGCATCTCCCCCTACTGTACACCACCAAAAGTG |
| | CAATCTATACGTAGAGGGGGATGACATGTGGTGGTTTTCAC |
| Pthxo1-3 (SEQ ID NO: 7) | GATATGCATCT CCCCC TACTGTACACCACCAA |
| | CTATACGTAGAGGGGGATGACATGTGGTGGTT |
| Pthxo1-4 (SEQ ID NO: 8) | GATATGCATCT CCCCC TACTGTACACCACCAAA |
| | CTATACGTAGAGGGGGATGACATGTGGTGGTTT |
| Pthxo1-5 (SEQ ID NO: 9) | AGATATGCATCTCCCCC TACTGTACACCACCAA |
| | TCTATACGTAGAGGGGGATGACATGTGGTGGTT |
| Pthxo1-6 (SEQ ID NO: 10) | AGATATGCATCT CCCCC TACTGTACACCACCAAA |
| | TCTATACGTAGAGGGGGATGACATGTGGTGGTTT |
| Pthxo1-7 (SEQ ID NO: 11) | AGATATGCATCT CCCCC TACTGTACACCACCAAAA |
| | TCTATACGTAGAGGGGGATGACATGTGGTGGTTTT |
| Pthxo1-8 (SEQ ID NO: 12) | TAGATATGCATCT CCCCC TACTGTACACCACCAAAA |
| | ATCTATACGTAGAGGGGGATGACATGTGGTGGTTTT |
| Pthxo1-9 (SEQ ID NO: 13) | TAGATATGCATCT CCCCC TACTGTACACCACCAAAAG |
| | ATCTATACGTAGAGGGGGATGACATGTGGTGGTTTTC |
| Pthxo1-10 (SEQ ID NO: 14) | TTAGATATGCATCT CCCCC TACTGTACACCACCAAAAG |
| | AATCTATACGTAGAGGGGGATGACATGTGGTGGTTTTC |
| Pthxo1-11 (SEQ ID NO: 15) | TTAGATATGCATCT CCCCC TACTGTACACCACCAAAAGT |
| | AATCTATACGTAGAGGGGGATGACATGTGGTGGTTTTCA |
| Pthxo1-12 (SEQ ID NO: 16) | TAGATATGCATCT CCCCC TACTGTACACCACCAAAA |
| | ATCTATACGTAGAGGGGGATGACATGTGGTGGTT |
| Pthxo1-13 (SEQ ID NO: 17) | TAGATATGCATCT CCCCC TACTGTACACCACCAAAA |
| | ATCTATACGTAGAGGGGGATGACATGTGGTGGTTT |
| Pthxo1-14 (SEQ ID NO: 18) | TAGATATGCATCT CCCCC TACTGTACACCACCAAAA |
| | AATCTATACGTAGAGGGGGATGACATGTGGTGGTTT |
| Pthxo1-15 (SEQ ID NO: 19) | TAGATATGCATCT CCCCC TACTGTACACCACCAAAA |
| | CAATCTATACGTAGAGGGGGATGACATGTGGTGGTT |
| Pthxo1-16 (SEQ ID NO: 20) | GTTAGATATGCATCT CCCCCTACTGTACACCACCAAAAGT |
| | CAATCTATACGTAGAGGGGGATGACATGTGGTGGTTTT |
| Pthxo1-17 (SEQ ID NO: 21) | GTTAGATATGCATCT CCCCCTACTGTACACCACCAAAAGT |
| | CAATCTATACGTAGAGGGGGATGACATGTGGTGGTTTTC |
| Pthxo1-18 (SEQ ID NO: 22) | TTAGATATGCATCT CCCCC TACTGTACACCACCAAAAGT |
| | CAATCTATACGTAGAGGGGGATGACATGTGGTGGTTTTC |
| Pthxo1-19* (SEQ ID NO: 23) | TAGATATGCATCT CCCCC TACTGTACACCACCAAAAGT |
| | CAATCTATACGTAGAGGGGGATGACATGTGGTGGTTTT |
| Pthxo1-20 (SEQ ID NO: 24) | GTTAGATATGCATCT CCCCC TACTGTACACCACCAAAAG |
| | CAATCTATACGTAGAGGGGGATGACATGTGGTGGTTTT |
| Pthxo1-21 (SEQ ID NO: 25) | GTTAGATATGCATCT CCCCC TACTGTACACCACCAAAAG |
| | CAATCTATACGTAGAGGGGGATGACATGTGGTGGTTTTC |
| Pthxo1-22 (SEQ ID NO: 26) | TTAGATATGCATCT CCCCC TACTGTACACCACCAAAAG |
| | CAATCTATACGTAGAGGGGGATGACATGTGGTGGTTTTC |
| Pthxo1-23 (SEQ ID NO: 27) | TAGATATGCATCT CCCCC TACTGTACACCACCAAAAG |
| | CAATCTATACGTAGAGGGGGATGACATGTGGTGGTTTT |
| Pthxo1-24 (SEQ ID NO: 28) | GATATGCATCT CCCCC TACTGTACACCACCAAAA |
| | ATCTATACGTAGAGGGGGATGACATGTGGTGGTT |
| Pthxo1-25 (SEQ ID NO: 29) | AGATATGCATCT CCCCC TACTGTACACCACCAAAA |
| | ATCTATACGTAGAGGGGGATGACATGTGGTGGTT |
| Pthxo1-26 (SEQ ID NO: 30) | AGATATGCATCT CCCCC TACTGTACACCACCAAAA |
| | ATCTATACGTAGAGGGGGATGACATGTGGTGGTTT |
| Pthxo1-27 (SEQ ID NO: 31) | TAGATATGCATCT CCCCC TACTGTACACCACCAAAAT |
| | AATCTATACGTAGAGGGGGATGACATGTGGTGGTTTT |
| Pthxo1-28 (SEQ ID NO: 32) | TAGATATGCATCT CCCCC TACTGTACACCACCAAAAGGT |
| | CCAATCTATACGTAGAGGGGGATGACATGTGGTGGTTTT |
| Pthxo1-29 (SEQ ID NO: 33) | AGATATGCATCT CCCCC TACTGTACACCACCAAAGTT |
| | CAATCTATACGTAGAGGGGGATGACATGTGGTGGTTT |
| Pthxo1-30 (SEQ ID NO: 34) | AGATATGCATCT CCCCC TACTGTACACCACCAAAAGTT |
| | CAATCTATACGTAGAGGGGGATGACATGTGGTGGTTTT |
| Pthxo1-31 (SEQ ID NO: 35) | TAGATATGCATCT CCCCC TACTGTACACCACCAAAAGTG |
| | CACATCTATACGTAGAGGGGGATGACATGTGGTGGTTTT |
| Pthxo1-32 (SEQ ID NO:) 36 | TAGATATGCATCT CCCCC TACTGTACACCACCAAAAGGT |
| | CAATCTATACGTAGAGGGGGATGACATGTGGTGGTTTTC |

The starred construct (Pthxo1-19) gave the best crystals, used in this study.
The target site is indicated (underline); the variable ends are in bold.
Subsequent attempts to optimize the ends and overhangs did not improved overall diffraction quality.

### Example 2

### Engineered TAL Effector Proteins with Enhanced DNA Targeting Capacity

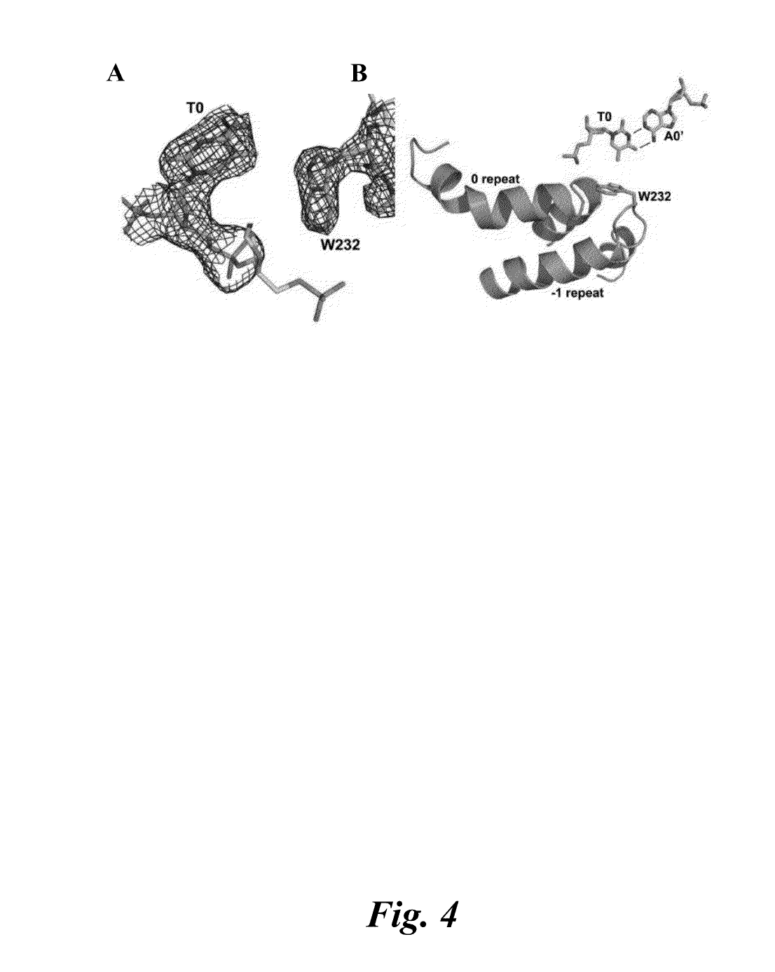

Functional TAL effector binding sites in nature are uniformly initiated by a 5′ thymine that precedes the nucleotide sequence specified by the TAL effector repeats. The amino acid sequence that immediately precedes the repeat region bears some similarity to the consensus amino acid sequence of the repeats. This similarity suggested that this sequence may form a cryptic “0^{th}” repeat that specifies the thymine. In Example 1 we determined the 3D structure of TAL effector PthXo1 bound to its target DNA. The structure revealed N-terminal to the canonical repeats, not one but two degenerate repeat folds that appear to cooperate to specify the thymine that is consistently found immediately preceding the RVD-specified sequence of TAL effector targets (FIG. 4). Residues 221 to 288 form two partially folded regions that loosely recapitulate the topology of their neighboring repeats. We have designated these as the 0^{th} and −1^{st} repeats. Residues 221 to 239 and residues 256 to 273 each form a helix and an adjoining loop that resembles helix 1 and the RVD loop in the canonical repeats; the remaining residues in each region are disordered. The ordered portions of those two N-terminal regions converge near the 5′ thymine base, with the indole ring of tryptophan 232 (in the −1^{st} repeat) making a van der Waals contact with the methyl group of that base. That tryptophan, as well as the surrounding residues, is highly conserved across available, intact TAL effector sequences, providing an explanation for the ubiquity of the thymine in TAL effector binding sites. Interestingly, some custom TAL effectors designed to target sequences that happened to be preceded by a cytosine rather than a thymine did so efficiently. Though less favorable, the packing of tryptophan 232 would be expected to accommodate this substitution, but not substitutions of a purine, which so far have not been observed in functional targets.

The current invention consists of amino acid modifications to the −1^{st} and 0^{th} repeats that allow efficient binding to sites initiated by any of the four nucleosides, either of 2 nucleosides, or one of the four nucleosides specifically. The modifications and their predicted novel specificities are summarized in Table 3.

TABLE 3
−1 and 0^{th} repeat mutations to alter specificity for T at −1.

| −1 repeat | 0^{th} repeat | Intended |
|---|---|---|
| mutation(s) | mutation(s) | Specificity |
| none | KR*GG−> SNGGG | T |
| none | KR*GG−> SHDGG | C |
| none | KR*GG−> SNIGG | A |
| none | KR*GG−> SNNGG | G/A |
| none | KR*GG−> KNGGG | T |
| none | KR*GG−> KHDGG | C |
| none | KR*GG−> KNIGG | A |
| none | KR*GG−> KNNGG | G/A |
| none | KRGG−> NGGG | T |
| none | KRGG−> HDGG | C |
| none | KRGG−> NIGG | A |
| none | KRGG−> NNGG | G/A |
| none | KRGG−> KNGG | T |
| none | KRGG−> KHDG | C |
| none | KRGG−> KNIG | A |
| none | KRGG−> KNNG | G/A |
| QWS−>QAS | None | A/C/G/T* |
| QWS−>QAS | KR*GG−> SNGGG | T |
| QWS−>QAS | KR*GG−> SHDGG | C |
| QWS−>QAS | KR*GG−> SNIGG | A |
| QWS−>QAS | KR*GG−> SNNGG | G/A |
| QWS−>QAS | KR*GG−> KNGGG | T |
| QWS−>QAS | KR*GG−> KHDGG | C |
| QWS−>QAS | KR*GG−> KNIGG | A |
| QWS−>QAS | KR*GG−> KNNGG | G/A |
| QWS−>QAS | KRGG−> NGGG | T |
| QWS−>QAS | KRGG−> HDGG | C |
| QWS−>QAS | KRGG−> NIGG | A |
| QWS−>QAS | KRGG−> NNGG | G/A |
| QWS−>QAS | KRGG−> KNGG | T |
| QWS−>QAS | KRGG−> KHDG | C |
| QWS−>QAS | KRGG−> KNIG | A |
| QWS−>QAS | KRGG−> KNNG | G/A |
| QW*S−>Q NGS | None | T |
| QW*S−>Q HDS | None | C |
| QW*S−>Q NIS | None | A |
| QW*S−>Q NNS | None | G/A |
| QWS−> NGS | None | T |
| QWS−> HDS | None | C |
| QWS−> NIS | None | A |
| QWS−> NNS | None | G/A |
| QWS−> QNG | None | T |
| QWS−> QHD | None | C |
| QWS−> QNI | None | A |
| QWS−> QNN | None | G/A |
| QWS−>QCS | None | |
| QWS−>QDS | None | |
| QWS−>QES | None | |
| QWS−>QFS | None | |
| QWS−>QGS | None | |
| QWS−>QHS | None | |
| QWS−>QIS | None | |
| QWS−>QKS | None | |
| QWS−>QLS | None | |
| QWS−>QMS | None | |
| QWS−>QNS | None | |
| QWS−>QPS | None | |
| QWS−>QQS | None | |
| QWS−>QRS | None | |
| QWS−>QSS | None | |
| QWS−>QTS | None | |
| QWS−>QVS | None | |
| QWS−>QYS | None | |

Residues functioning as candidate RVDs are bolded.
*indicates that an amino acid will be inserted at this position.

### Example 3

### Introduction

Functional TAL effector binding sites observed in nature to date are uniformly initiated by a 5′ thymine (T), or in one case by a 5′ cytosine (C), that precedes the nucleotide sequence specified by the TAL effector repeats (referred to as position 0). This thymine has been demonstrated to be required for full TAL effector activity and is presumed to be important for full affinity binding of the effector to the DNA. The requirement for the T represents a significant constraint on the design of custom TAL effector-based constructs for DNA targeting. The recently determined structure of TAL effector PthXo1 bound to its target DNA revealed the presence of two cryptic repeats (called the −1^{st} repeat and the 0^{th} repeat) located N-terminal to the canonical repeats. These cryptic repeats appear to cooperate to specify the 0 position T (FIG. 4). In particular, tryptophan 232 in the −1^{st} repeat packs the space between the effector and the DNA in a way that makes the presence of the T energetically favorable through specific van der Waals contacts with the tryptophan. Tryptophan 232 and the surrounding residues are highly conserved across available TAL effector sequences and may provide an explanation for the ubiquity of the position 0 thymine preceding TAL effector binding sites.

The previous example described a conceived invention consisting of amino acid modifications to the −1^{st} and 0^{th} repeats allowing efficient binding to sites initiated by any of the four nucleosides, either of 2 nucleosides, or one of the 4 nucleosides specifically. Here, we show that specific amino acid substitutions for tryptophan 232 in the −1^{st} repeat of TAL effector PthXo1 alter the specificity of the TAL effector for the nucleotide at the 0 position, and alter the activity level of the TAL effector, which likely reflects a change in affinity. In particular, we have now generated substitutions that completely relax specificity, substitutions that relax specificity and double activity, and substitutions that change specificity and increase activity, among others.

### Methods:

We generated a full length TAL effector construct containing the PthXo1 repeat sequence. Site directed mutagenesis was used to substitute all 19 other amino acids individually for tryptophan 232. The modified PthXo1 TAL effectors were cloned into binary expression vector pGWB5. The naturally occurring target of PthXo1 (UptPthXo1) was cloned into binary GUS reporter vector pGWB3. The thymine at position 0 was modified to adenine, cytosine, or guanine using site directed mutagenesis. PthXo1 constructs and each of the four target GUS reporter constructs were transformed into Agrobacterium and co-inoculated into the leaves 6-8 week old Nicotiana benthamiana plants. After 48 hours, GUS activity was measured using a fluorometric assay. Each experiment included a minimum of 4 replicates.

### Results and Discussion.

GUS activity for 17 TAL effector constructs on each of the four targets is shown in FIG. 9. Several −1^{st} repeat substitutions for Tryptophan 232 show altered specificities at position 0 as well as increases in activity relative to wild type PthXo1 on the target preceded by thymine. Substitutions that we predict will be particularly useful include glutamine (Q), which increases activity and alters specificity so that a cytosine is preferred, threonine (T), which increases activity and eliminates all specificity), proline (P), which increases activity and partially relaxes specificity to allow guanine or thymine at position 0, and asparagine (N) which retains wild-type activity and eliminates all specificity at the 0 position.

Our results demonstrate that substituting the −1^{st} repeat 232 tryptophan with other amino acids can alter the requirement for thymine at position 0 and increase TAL effector binding activity. The results are novel because they are the first to demonstrate that the −1^{st} repeat of TAL effectors can be engineered to increase the activity of these proteins and to change their requirement for an initiating thymine at their DNA binding site. They are useful because eliminating the requirement for thymine at position 0 broadens the targeting range of custom TAL effectors to virtually any sequence of nucleotides in DNA, enhancing the utility of TAL effectors as tools for custom gene regulation, genome engineering, and other DNA targeting applications. Several of the substitutions also show significant increases in activity, which will be beneficial to all of the applications listed. These results will be useful commercially, as they represent a significant improvement to current TAL-effector base d protein tools.

### Example 4

### Amino Acid Substitutions for Tryptophan 232 in TAL Effectors Relax the Requirement for Thymine at the 0th Position of the Binding Site

TAL effectors are transcription factors translocated from plant pathogenic Xanthomonas spp. into plant cells during infection. Target sequence specificity is defined by polymorphic repeats in a central domain of the proteins: each repeat independently specifies a single nucleotide in the binding site. Due to this modularity, the repeat region can be customized to recognize DNA sequences of choice, so TAL effectors have increasingly been used as DNA targeting domains in engineered proteins for genome editing and targeted gene regulation. TAL effectors can be easily targeted to almost any sequence of interest; however, the binding site must be directly preceded by a thymine at the 0th position for maximal activity. Structural information suggests that this is a binding requirement encoded by a tryptophan at position 232 (W232), which resides in a cryptic repeat immediately N-terminal to the repeat region of the protein. We generated TAL effectors with single amino acid substitutions for W232 and tested them in a transcriptional activation assay and a DNA binding assay. We identified several substitutions that relax the requirement for thymine in the activity assay and one, a proline substitution, W232P, that relaxed specificity and increased affinity in the DNA binding assay. Additionally, we characterized the N-terminal portion of Ralstonia TAL effector-like proteins by substituting one into a Xanthomonas TAL effector. In this context, the Ralstonia N-terminus specified a guanine at the 0th position of the target and reduced overall activity in the transcriptional activation assay. The amino acid in these proteins at the position corresponding to 232 is an arginine. Substitution of tryptophan or any of several other tested amino acids at this position destroyed activity of the chimeric protein, indicating structural and functional differences of the Ralstonia N terminus from the Xanthomonas one. Our results demonstrate that manipulation of the N-terminal amino acid sequences of TAL effectors can relax or alter their requirement for a thymine preceding their repeat-specified target site.

Transcription activator-like (TAL) effectors from the plant-pathogenic bacterial genus Xanthomonas have generated considerable interest as tools for site-specific DNA modifications. In nature, TAL effectors function as transcription factors. They are secreted from the pathogen into the host plant cell via the type three secretion system (T3SS). Once inside the cell, they localize to the nucleus where they bind to effector-specific DNA sequences called UPT boxes (for “upregulated by TAL”) and activate genes necessary for bacterial multiplication and survival. Each TAL effector's specific binding site is determined by a central repeat region (CRR), which is composed of a variable number of 33-35 amino acid tandem repeats. Repeats are nearly identical, with variation centered at amino acids 12 and 13 (termed the repeat-variable diresidue, or RVD). The sequence of RVDs corresponds directly to the DNA binding sites, with each repeat/RVD specifying a single binding site nucleotide. The discovery of this TAL-effector nucleotide binding code has made it possible to engineer custom TAL effectors with novel binding specificities. TAL effectors with customized binding domains have been successfully used to activate specific genes, using either the native TAL activation domain at the C-terminus of the TAL effector, or replacing it with the VP16 herpes simplex virus activation domain or its tetrameric derivative VP645-8. Targeted gene repression using TAL effector-based repressor fusions has also been demonstrated. TAL effector fusions to the FokI endonuclease (TAL effector nucleases or TALENs) have been used to create targeted double strand breaks (DSBs) in DNA. DSBs are subsequently repaired by one of two pathways. Non-homologous end joining (NHEJ) frequently results in insertions or deletions, and can be used to disrupt a gene. Homologous recombination (HR) inserts a DNA template of similar sequence into the break; this can be used to edit a portion of the gene. TALEN-mediated NHEJ and HR have been demonstrated in a wide variety of cell types and organisms. Custom TALENs are easier to design and target than other sequence-specific nucleases (such as zinc finger nucleases) that have been used for similar genome editing.

Thus far, TAL effector repeats appear to behave modularly, with no neighbor or context effects reported. However, the design and targeting of custom TAL effectors is constrained by a requirement for the TAL effector binding site to be directly preceded by a thymine (T) at the 5′ end (known as the 0th position). This 0th position thymine is almost uniformly conserved in all known TAL effector binding sites in nature. The only known exception is the target of TalC from Xanthomonas strain BAI3, whose target is preceded by a 5′ cytosine (C). Replacing the 0th position T with another base has been shown to dramatically reduce or eliminate TAL effector-driven activation of reporter genes and DNA binding. Custom TAL effectors have typically been designed to target sites preceded by a 0th position T. Although functional TALENs targeting sites preceded by other nucleotides have been reported, direct comparisons of activity on targets preceded by a thymine were not.

Thus, the requirement for a T preceding the TAL effector binding site appears to represent the most significant constraint on the design of custom TAL effectors and TAL effector-based proteins. Recently, the crystal structures of TAL effectors PthXo1 from Xanthomonas oryzae pv. oryzae and custom TAL effector dHAX3 bound to their respective DNA targets revealed the structural basis for TAL effector-DNA binding. Additionally, the PthXo1 structure revealed two cryptic repeats (called the 0th and −1st repeats) located immediately N-terminal to the canonical repeats encoding the binding site. Residue 232 of the PthXo1, a tryptophan (W) located in the −1st repeat, was shown to be in close proximity to the 0th position thymine preceding the binding site and to specific make van der Waals contacts with the thymine that are predicted to make that base the most energetically favorable at that position. Tryptophan 232 and the surrounding residues are absolutely conserved in known Xanthomonas TAL effector sequences. Therefore, it is probable that tryptophan 232 plays a significant role in specifying the 0th position thymine. Here using both transcriptional activation and DNA binding assays, we show that tryptophan 232 specifies the thymine at position 0 of the target. In the transcriptional activation assay, using PthXo1 variants with all 19 possible amino acids substitutions at position 232 of the protein, we identified several substitutions that either changed or relaxed specificity for the thymine at position 0 of the target while maintaining activity comparable to the wild type protein, and some that increased activity. Using a custom TAL effector that expreewd and purified better than PthXo1, we tested a subset of these substitutions in a DNA binding assay and found one, W232P, that relaxed specificity for thymine entirely, allowing any base, with a slight preference for guanine, and no appreciable decrease in affinity relative to the wild type on the target with thymine. Additionally, we characterized the N-terminal portion of Ralstonia TAL effector-like proteins by substituting one into PthXo1. In this context, the Ralstonia N-terminus specified a guanine at the 0th position of the target and showed reduced overall activity in the transcriptional activation assay relative to the native PthXo1. The amino acid at the position corresponding to 232 is an arginine. Substitution of tryptophan or any of several other tested amino acids at this position destroyed activity of the chimeric protein, indicating structural and functional differences of the Ralstonia N terminus from the Xanthomonas one. Our results demonstrate that manipulation of the N-terminal amino acid sequences of TAL effectors can relax or alter their requirement for a thymine preceding their repeat-specified target site while maintaining affinity and in some cases increasing activity. In addition to elucidating the function of the cryptic −1st repeat, these findings enable targeting custom TAL effectors and TAL effector based proteins such as TALENs to sequences preceded by any base, rather than only thymine.

### Results

### Single Amino Acid Substitutions for Tryptophan 232 Affect Activity of PthXo1 and its Requirement for Thymine at the 0th Position of its Target.

To test the effects of substitutions for the tryptophan at position 232 on TAL effector activity and base preference at the 0th position of the target, we used Agrobacterium-mediated transient expression of PthXo122 (Table 4) and variants with all 19 other amino acids in place of the tryptophan (FIG. 10A) to drive a codelivered GUS reporter under control of the Bs3 promoter 23 modified to contain the natural target of PthXo1 3 (designated as UptPthXo1, for Up-regulated by TAL effector PthXo1, shown in FIG. 13) preceded by thymine, adenine, cytosine, or guanine, in Nicotiana benthamiana leaves. The PthXo1 variants were expressed from constructs driven by the 35S promoter. GUS activity was measured after 48 hours. Results are shown in FIG. 10B.

TABLE 4
PthXo1 RVD sequence and target sequences. Shown are the RVD sequence of PthXo1,
the nucleotide sequence of its natural target from the promoter of rice gene
Os8N31, and a target optimized according to the TAL effector DNA binding code.
Mismatches (RVD nucleotide pairs that do not conform to the pairings NI-A,
HD-C, NN-G/A, NG-T) in the natural target are highlighted in grey.

| | 1 | 2 | 3 | 4 | 5 | 6 | 7 | 8 | 9 | 10 | 11 | 12 | 13 |
|---|---|---|---|---|---|---|---|---|---|---|---|---|---|
| PthXo1 | NN | HD | NI | HG | HD | NG | N* | HD | HD | NI | NG | NG | NI |
| Natural | G | C | A | T | C | T | C | C | C | [symbol] | [symbol] | T | A |
| Optimized | G | C | A | T | C | T | C | C | C | A | T | T | A |

| | 14 | 15 | 16 | 17 | 18 | 19 | 20 | 21 | 22 | 23 | 23.5 |
|---|---|---|---|---|---|---|---|---|---|---|---|
| PthXo1 | HD | NG | NN | NG | NI | NI | NI | NI | N* | NS | N* |
| Natural | C | T | G | T | A | [symbol] | A | [symbol] | C | A | C |
| Optimized | C | T | G | T | A | A | A | A | C | A | C |

PthXo1 on targets preceded by any base other than thymine showed reduced activity relative to that on the target preceded by thymine. Several amino acid substitutions reduced or eliminated activity on all target variants compared to the wild type PthXo1. A few substitutions enhanced activity and/or yielded greatest activity on a target preceded by a base other than thymine, representing altered specificity relative to unmodified PthXo1. Key substitutions included glutamine (W232Q), which increased activity and altered specificity so that a cytosine is preferred; threonine (W232T), which increased activity and eliminated all specificity to allow any of the four nucleotides at position 0; asparagine (N), which retained wild-type activity levels and eliminated all specificity at the 0 position; and proline (W232P), which relaxed specificity so that activity on each target variant was as good or better than activity of unmodified PthXo1 on the target preceded by thymine. W232P showed highest activity on the target preceded by guanine, and that activity was higher than the activity of PthXo1 on its target preceded by thymine. Substitution of arginine (W232R), which is the amino acid present at the analogous position in Ralstonia TAL effector-like proteins (see below), resulted in slightly reduced overall activity relative to unmodified PthXo1, and highest activity on the target preceded by cytosine.

### Selected Amino Acid Substitutions for Tryptophan 232 Alter DNA Binding Affinity of a Custom TAL Effector and its Base Preference at Position 0 of the Target

To test whether the changes in activity and base preference at the 0th position of the target observed in the transcriptional activation assay correlate with changes in target binding affinity, we carried out electrophoretic mobility shift assays. Expressed in E. coli and purified, recombinant PthXo1 proved to be unstable in these assays. We therefore generated a custom TAL effector, TAL868 with 15 RVDs, that was stable and suitable. TAL868 and variants with substitutions W232N, W232P, W232Q, W232R, and W232T were assayed over a range of concentrations for binding to double stranded DNA fragments containing the code-specified target preceded by thymine, adenine, cytosine, or guanine. A scrambled target was included as a negative control. Results are shown in FIG. 11. Consistent with the results of the PthXo1 activity assay, substitutions of any base for the thymine at position 0 of the target reduced the affinity of the unmodified TAL effector for the DNA. In contrast to the activity assay results with PthXo1, substitutions W232Q, W232R, and W232T in TAL868 caused a reduced apparent affinity on all target variants. W232N caused a reduction in affinity for the target preceded by T and little effect on the affinity of the protein for the other target variants. W232P however, in agreement with the transcriptional activity assay results, showed binding to the target preceded by thymine comparable to that of the unmodified effector, and binding to each target variant that was roughly equivalent to the binding on the target preceded by thymine. These results demonstrate that the W232P substitution, in different TAL effector proteins and in different assays, relaxes specificity for the thymine at position 0 of the target while maintaining affinity and maintaining or increasing activity.

Substitution of the TAL Effector N-Terminal Region with that of a Ralstonia TAL Effector-Like Protein (RTL) Changes Specificity for the 0th Position of the Target to Guanine

TAL effector-like proteins are found in the soil-borne plant pathogenic bacterium Ralstonia solanacearum. Like Xanthomonas TAL effectors, these Ralstonia TAL effector-like proteins (RTLs) possess a CRR, although the RTL repeats are uniformly 35 amino acids in length and distinct in amino acid sequence. RTLs are known to be secreted into host plants by the bacterium's T3SS1. It is not known whether they function as transcriptional activators.

An alignment of three available complete predicted RTL amino acid sequences, RSc1815 (CAD15517.1) from R. solanacearum strain GMI1000, CAQ18687.1 from strain MolK2, and hpx17 (AB178011.1) from strain RS108527, 29 to the amino acid sequence of PthXo1 showed a high degree of sequence similarity in the regions directly N terminal to the CRR. Tryptophan 232 is replaced by an arginine, but the residues directly on either side are conserved (FIG. 14). Secondary structure predictions revealed that for all of the RTLs, the N terminus is likely to form six helices, with four helices corresponding approximately to the two 2-helix bundles that make up 0th and −1st repeats of PthXo1. Tryptophan 232 (PthXo1) and the equivalent arginines (RTLs) are centered on short (three and two amino acids, respectively) loops between the helices that form the −1st repeat (See FIG. 4B).

Because sequence alignments and secondary structure predictions indicated similar structures for the N terminal regions of PthXo1 and the RTLs, we tested whether the RTL N-terminal region could function similarly to the TAL effector one to specify the 0th position of the target. Naturally occurring targets for RTLs are currently unknown, and many RTL repeat sequences contain RVDs with unknown specificity. Therefore, we tested the RTL's N-terminal function as part of a chimeric construct with the CRR and C-terminal portions of PthXo1 (FIG. 12). We called this protein RScPthXo1. In the transcriptional activation assay against the UptPthXo1 GUS reporter construct with thymine preceding the PthXo1 RVD specified target, and variants with either adenine, cytosine, or guanine in place of the thymine, RScPthXo1 showed activity that was greatly reduced relative to wild type PthXo1, and a change in preference for the 0th position base, with the highest activity being on the target preceded by guanine rather than thymine. Substitution of tryptophan for the arginine in the corresponding position of this protein was not sufficient to revert the activity level and specificity of this protein to that of PthXo1. In fact that substitution further reduced activity across all target variants. Substitutions of proline, glutamine, threonine, or asparagine for the arginine rendered the protein essentially inactive.

### Discussion

We have shown here that the cryptic −1st repeat of Xanthomonas TAL effectors accounts for the requirement for thymine directly preceding the RVD-specified TAL effector target site. We show that by altering the −1 repeat residue tryptophan 232, which we previously predicted to interact with and specify the 0th position thymine, the specificity for thymine at this position can be altered. The majority of substitutions reduced TAL effector activity. However, specific amino acid substitutions were identified that relaxed specificity for the 0th position base or altered the specificity to other nucleotides while maintaining or improving activity. Assays of the effects of a subset of these substitutions on TAL effector affinity for the target with thymine at position 0, as well as for variants with adenine, cytosine, or guanine at position 0, were also carried out, using a different TAL effector form that used in the activity assays. These experiments yielded results that partially conflicted with the activity assay results, with most substitutions reducing apparent affinity on all target variants. The conflicting results could have arisen due to differential effects in the different proteins, or, in contrast to expectation, activity may in fact not correlate with affinity, or some substitutions may affect activity and affinity independently, or some combination. Substitution of proline for the tryptophan was an exception however. It yielded results in the affinity assay consistent with those from the activity assay, and indicated that in different proteins in different experimental contexts, this substitution relaxes the requirement for thymine at the 0th position of the target while maintaining or improving activity and affinity.

We showed that in a chimeric context with the CRR and C-terminal region of a TAL effector, the N-terminal region of a TAL effector-like protein from Ralstonia solanacearum, which shows similarity to the TAL effector N-terminus in its sequence and predicted secondary structure, functions similarly to specify the 0th position, but causes a large reduction in activity and a shift in specificity for this base from thymine to guanine The reduced activity suggests that specific interactions may take place between the cryptic repeats and the repeats in the CRR of a native TAL effector that are poorly replicated in the chimera. Or, the activity may reflect that of native RTLs, which has not yet been reported. Interestingly, substitutions for the arginine in the RTL N terminal region that corresponds in location to tryptophan 232 in TAL effectors, with tryptophan or any of several other selected amino acids, drastically reduced activity, so that any reversion or change in specificity was not reliably detectable.

Our results demonstrate that manipulation of the N-terminal amino acid sequences of TAL effectors can relax or alter their requirement for a thymine preceding their repeat-specified target site. In particular, we identified at least one substitution, W232P that can be used in TAL effectors and TAL effector fusion proteins to enhance their targetability by removing the requirement for a thymine to precede the RVD-specified nucleotide sequence, while maintaining or improving activity and affinity. Further testing of other substitutions in different TAL effector proteins and different experimental contexts promises to reveal some that reliably alter specificity to other nucleotides to allow, in combination with an unmodified TAL effector, distinction of targets that differ only at the 0th position and limiting the potential for off-target binding. Further study also can be expected to clarify the relationship between activity and affinity and the contribution of position 232 to each of these.

### Methods

### GUS Reporter Assays.

To generate the PthXo1 TAL effector plasmid for GUS assays (called pAH236), the SphI fragment containing the repeat region of TAL effector PthXo1 (clone obtained from Bing Yang, Iowa State University) was cloned into the SphI site of plasmid pCS466 31. pCS466 is derived from the Gateway entry vector pCR8-GW (Invitrogen). It contains a truncated form of the Xoc BLS256 tal1c gene, from which the SphI fragment containing the DNA binding domain has been removed. This PthXo1 gene was then recombined into Gateway binary vector pGWB532. The recombination was done using Gateway LR Clonase (Invitrogen) according to the manufacturer's instructions. To make single amino acid substitutions for W232, the surrounding region of pAH236 was amplified via PCR using a forward primer crossing the Not1 site and a reverse primer crossing the StuI site and introducing the mutation and a silent Xho1 site to facilitate screening. The same forward primer was used for all substitutions. Primers are listed in Table 5. PCR products were digested with NotI (New England Biolabs, Inc.) and StuI (New England Biolabs, Inc.) and cloned into the NotI/StuI site of pAH236 using standard techniques. Expression of the TAL effectors is driven by the 35S promoter.

TABLE 5
Primers used for pAH236
tryptophan 232 substitutions.

| Muta- | Direc- | Oli- | |
|---|---|---|---|
| tion | tion | go | Sequence |
| all | Fwd. | 885 | 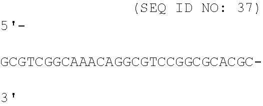 |
| W -> A | Rev. | 886 | 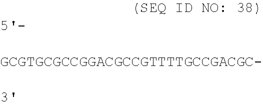 |
| W -> F | Rev. | 919 | 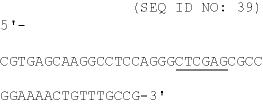 |
| W -> L | Rev. | 920 | 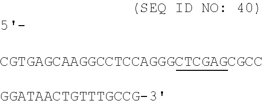 |
| W -> I | Rev. | 921 | 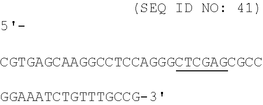 |
| W -> M | Rev. | 922 | 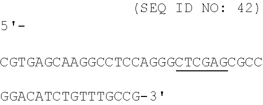 |
| W -> V | Rev. | 923 | 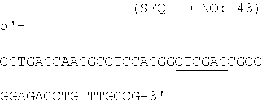 |
| W -> S | Rev. | 924 | 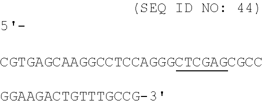 |
| W -> P | Rev. | 925 | 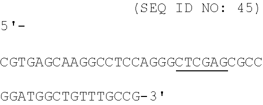 |
| W -> T | Rev. | 926 | 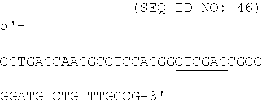 |
| W -> Y | Rev. | 927 | 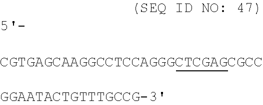 |
| W -> H | Rev. | 928 | 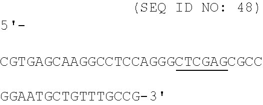 |
| W -> Q | Rev. | 929 | 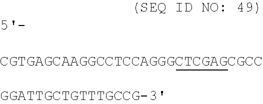 |
| W -> N | Rev. | 930 | 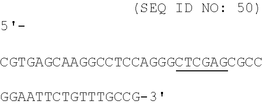 |
| W -> K | Rev. | 931 | 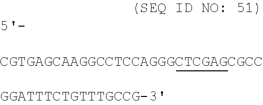 |
| W -> D | Rev. | 932 | 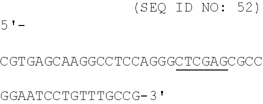 |
| W -> E | Rev. | 933 | 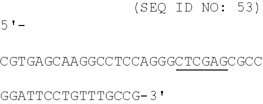 |
| W -> C | Rev. | 934 | 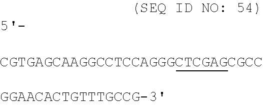 |
| W -> R | Rev. | 935 | 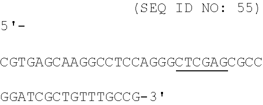 |
| W -> G | Rev. | 936 | 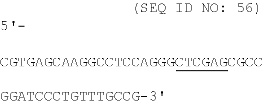 |

Codons introducing W232 substitutions are highlighted in grey.
The silent Xho1 site is underlined.

For PthXo1 GUS reporter constructs, the 343 base pair 5′ region of the Bs3 promoter was PCR amplified as previously described 16. The PCR product was cloned into the Gateway vector pCR8 (Invitrogen) and site directed mutagenesis was used to introduce an Asc1 site upstream of the naturally occurring AvrBs3 binding site. This modified promoter was recombined using LR Clonase II (Invitrogen) into the Gateway GUS reporter vector pGWB5 upstream of the GUS gene. Single stranded DNA oligos containing the UptPthXo1 preceded by A, C, G, or T and AscI-compatible sticky ends were annealed and cloned into the AscI site to create UptA, UptC, UptG, and UptT, respectively. Oligos used are summarized in Table 6.

TABLE 6
Annealed single-stranded DNA oligos for GUS assay reporter constructs.

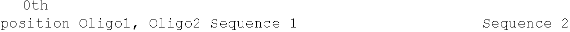

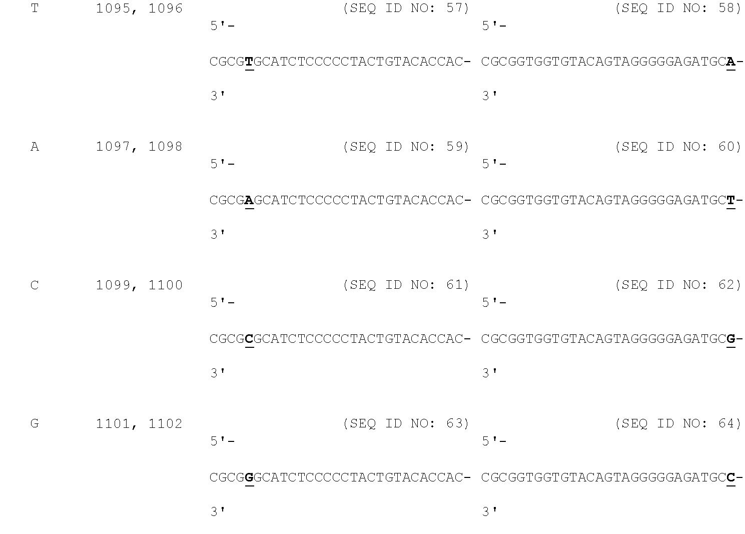

The 0^{th} position immediately preceding the binding site is in underlined.
The PthXo1 binding site is highlighted in grey.

To construct our chimeric TAL effector RScPthXo1 the Ralstonia TAL effector-like N-terminal sequence was PCR-amplified with Phusion DNA polymerase (Thermo Fisher Scientific, Waltham, Mass., USA) from GMI1000 genomic DNA with forward primer P1178 (5′-TTGCATGTAAATAGGAGGTGCACCATGAGAATAGGCAAATCAAG-3; which added sequence upstream of the start codon to match that of the Xanthomonas TAL effector expression constructs (SEQ ID NO:65)) and P1179 (5′-GAGACTCGTCTCGGCACGCGTGAGCTTCC-3; which added a downstream Esp3I restriction site for cloning (SEQ ID NO:66)). This amplicon was tailed with 3′ adenine residues using Taq Polymerase (Thermo Fisher Scientific, Waltham, Mass., USA) and cloned into the pCR8/GW/TOPO TA vector (Life Technologies, Grand Island, N.Y., USA). The resulting plasmid was digested with Esp3I and EcoRV restriction enzymes and ligated to the BanI/EcoRV restriction enzyme fragment of pAH103 to produce the full-length, Ralstonia/Xanthomonas chimeric TAL effector in the Gateway entry vector (pAH410). Each −1 repeat amino acid substation variant of the chimeric effector was generated by PCR-amplification with Phusion DNA polymerase of a portion of the Ralstonia N-terminus using forward primer P1178 and each of five reverse primers that generated five different amino acid substitutions at the −1 repeat (tryptophan (SEQ ID NO:67): 5′-CGGGTAGCAGCGCTTGCAGCGCCAGGTCACCCGACCACTGC-3; threonine (SEQ ID NO:68): 5′-CGGGTAGCAGCGCTTGCAGCGCCAGGTCACCCGATGTCTGC-3′; glutamine (SEQ ID NO:69): 5′-CGGGTAGCAGCGCTTGCAGCGCCAGGTCACCCGATTGCTGC-3; proline (SEQ ID NO:70): 5′-CGGGTAGCAGCGCTTGCAGCGCCAGGTCACCCGATGGCTGC-3; asparagine (SEQ ID NO:71): 5′-CGGGTAGCAGCGCTTGCAGCGCCAGGTCACCCGAATTCTGC-3′). Each amplicon was digested with AfeI and BsaI restriction enzymes and cloned into the AfeI/BsaI restriction enzyme digested pAH410. The chimeric effector and amino acid substitution variants were transferred to the binary expression vector pGWB5 via the Gateway LR II Clonase enzyme kit (Life Technologies).

For the GUS assays, TAL effector and reporter constructs were transformed into A. tumefaciens GV3101. Cells were grown overnight and diluted to OD600=0.8. Cells carrying a TAL effector construct were mixed 1:1 with cells carrying a GUS reporter construct. Mixed cells were infiltrated using a needle-less syringe onto the leaves of 6-8 week old Nicotiana benthamiana plants. Plants were grown in a growth room under standard conditions. After 48 hours, infiltrated leaf discs were ground in 300 μL GUS extraction buffer (50 mM sodium phosphate buffer (pH 7), 10 mM EDTA, 10 mM β-mercaptoethanol, 0.1% Triton X-100, 0.1% SDS) to extract the proteins. Following centrifugation, 100 μL supernatant was mixed with 90 μL assay buffer (GUS extraction buffer with 10 mM 4-methyl-umbelliferyl-β-D-glucuronide added) and incubated at 37° C. 10 μL of the reaction was stopped after one hour by adding 90 μL 0.2M sodium carbonate (pH 9.5). 360 nm (excitation) and 460 nm (emission) were measured in a plate reader. Protein amounts were quantified by Bradford assay (BioRad). Data are reported as the average of 4 leaf discs. For key constructs, experiments were repeated at least twice with similar results.

### Expression and Purification of Recombinant TAL Effector Protein

The bacterial expression vector pGEX6P2-TALE was created by ligating a Golden Gate compatible fragment with the NΔ152/C+63 architecture into pGEX6P2 (GE Healthcare). Variants with substitutions at W232 were created by site-directed mutagenesis (Quickchange II, Agilent). An array of 15 repeats with the RVD sequence (NI HD NN NG NG NI NI NG NN NN NI NI NN HD NG) was then cloned into each of these vectors using the golden gate method as previously described. Expression constructs encoding the TAL effector proteins were then transformed into Rosetta (DE3) pLysS cells (EMD Millipore) and selected on media containing carbenicillin (50 μg/ml) and chloramphenicol (30 μg/ml). 200 mL cultures were grown to log phase at 37° C. before induction for 3 hours with 1 mM IPTG. The cells were pelleted by centrifugation and lysed in GST lysis buffer (25 mM HEPES pH 7.4, 150 mM NaCl, 5 mM MgCl2, 130 μM CaCl2, 0.5% Triton X-100, 10% glycerol, 1 mM PMSF, 1 μg/mL Leupeptin, 100 nM Aprotinin, 1 μg/mL Pepstatin A). The lysates were treated with RNase A (20 μg/mL) and DNase I (10 U/mL), clarified by centrifugation (21,000×g, 10 minutes) and then loaded onto a column containing equilibrated Glutathione Sepharose (GE Healthcare). The columns were washed with GST lysis buffer and subsequently by cleavage buffer (50 mM Tris-HCl pH 8.0, 1 mM EDTA, 1 mM DTT, 10% glycerol). Elution of untagged purified TALE protein was performed by overnight incubation at 4° C. with PreScission protease (GE Healthcare). Purified TALE proteins were separated by electrophoresis and stained with Coomassie to determine the purity of the samples.

### Electrophoretic Mobility Shift Assay (EMSA)

Double stranded DNA substrates were prepared by annealing fluorescently tagged complementary oligos. Sequences for substrates used were 5-AACGTTAATGGAAGCT for UptA (SEQ ID NO:72), 5′-CACGTTAATGGAAGCT for UptC (SEQ ID NO:73), 5′-GACGTTAATGGAAGCT for UptG (SEQ ID NO:74), 5′-TACGTTAATGGAAGCT for UptT (SEQ ID NO:75), and 5′-TCGACGCTCAGGCAAC for the scrambled target (SEQ ID NO:76). The purified proteins were diluted into binding buffer (10 mM HEPES pH 7.6, 10% glycerol, 100 mM KCl, 10 mM MgCl2, 100 μM EDTA, 500 μM DTT, 15 ng/μL salmon sperm DNA, 30 ng/μL BSA) at varying concentrations with a fixed concentration of the labeled DNA substrate (20 nM). The reactions were incubated for 30 minutes at room temperature and then separated by electrophoresis on a 7% TBE-acrylamide gel. Detection of the labeled substrate was then performed on a fluorescent scanner (Storm 860, Molecular Dynamics).

### Multiple Sequence Alignments and Secondary Structure Predictions

The multiple sequence alignment (MSA) was done using ClustalW. Secondary structure predictions were done using Psipred.

### REFERENCES

S. Kay, S. Hahn, E. Marois, G. Hause, U. Bonas, A bacterial effector acts as a plant transcription factor and induces a cell size regulator. Science 318, 648 (Oct. 26, 2007).
P. Römer et al., Plant pathogen recognition mediated by promoter activation of the pepper Bs3 resistance gene. Science (New York, N.Y. 318, 645 (Oct. 26, 2007).
J. Boch et al., Breaking the code of DNA binding specificity of TAL-type III effectors. Science (New York, N.Y. 326, 1509 (Dec. 11, 2009).
M. J. Moscou, A. J. Bogdanove, A simple cipher governs DNA recognition by TAL effectors. Science (New York, N.Y. 326, 1501 (Dec. 11, 2009).
A. J. Bogdanove, S. Schornack, T. Lahaye, TAL effectors: finding plant genes for disease and defense. Curr. Opin. Plant Biology 13, 394 (August 2010).
J. Boch, U. Bonas, Xanthomonas AvrBs3 family-type III effectors: discovery and function. Ann. Rev. Phytopath. 48, 419 (Sep. 8, 2010).
A. J. Bogdanove, D. F. Voytas, TAL effectors: customizable proteins for DNA targeting. Science (New York, N.Y. 333, 1843 (Sep. 30, 2011).
J. C. Miller et al., A TALE nuclease architecture for efficient genome editing. Nature Biotech. 29, 143 (Dec. 22, 2011).
M. Christian et al., Targeting DNA double-strand breaks with TAL effector nucleases. Genetics 186, 757 (Jul. 26, 2010).
M. T. Murakami et al., The repeat domain of the type III effector protein PthA shows a TPR-like structure and undergoes conformational changes upon DNA interaction. Proteins: Structure, Function, and Bioinformatics DOI, 10.1002/prot.22846 (2010).
B. Yang, A. Sugio, F. F. White, Os8N3 is a host disease-susceptibility gene for bacterial blight of rice. Proc Natl Acad Sci USA 103, 10503 (Jul. 5, 2006).
L. D. D'Andrea, L. Regan, TPR proteins: the versatile helix. Trends Biochem Sci 28, 655 (December 2003).
R. Rohs et al., Origins of specificity in protein-DNA recognition. Annu Rev Biochem 79, 233 (2010).
A. N. Mak, A. R. Lambert, B. L. Stoddard, Folding, DNA recognition, and function of GIYYIG endonucleases: crystal structures of R.Eco29kI. Structure 18, 1321 (Oct. 13, 2010).
Z. Otwinowski, W. Minor, Processing of X-ray diffraction data collected in oscillation mode. Methods in Enzymology 276, 307 (1997).
C. Yanover, P. Bradley, Extensive protein and DNA backbone sampling improves structure-based specificity prediction for C2H2 zinc fingers. Nucleic Acids Res 39, 4564 (June 2011).
A. J. McCoy et al., Phaser crystallographic software. J Appl Crystallogr 40, 658 (Aug. 1, 2007).
A. J. Bogdanove et al., Two new complete genome sequences offer insight into host and tissue specificity of plant pathogenic Xanthomonas spp. J Bacteriol 193, 5450 (October 2011).
P. Romer et al., Promoter elements of rice susceptibility genes are bound and activated by specific TAL effectors from the bacterial blight pathogen, Xanthomonas oryzae pv. oryzae. New Phytol 187, 1048 (September 2010).
A. Leaver-Fay et al., ROSETTA3: an object-oriented software suite for the simulation and design of macromolecules. Methods Enzymol 487, 545 (2011).
P. D. Adams et al., PHENIX: a comprehensive Python-based system for macromolecular structure solution. Acta Crystallogr D Biol Crystallogr 66, 213 (February 2010).
P. Emsley, K. Cowtan, Coot: model-building tools for molecular graphics. Acta Crystallogr D Biol Crystallogr 60, 2126 (December 2004).
M. D. Winn, G. N. Murshudov, M. Z. Papiz, Macromolecular TLS refinement in REFMAC at moderate resolutions. Methods Enzymol 374, 300 (2003).
R. J. Laskowski, M. W. Macarthur, D. S. Moss, J. M. Thornton, PROCHECK: a program to check the stereochemical quality of protein structures. J. Appl. Crystall. 26, 283 (1993).
G. Vriend, WHATIF: a molecular modeling and drug design program. J. Mol. Graph. 8, 52 (1990).
P. W. Rose et al., The RCSB Protein Data Bank: redesigned web site and web services. Nucleic Acids Res 39, D392 (January 2011).
Scholze, H. & Boch, J. TAL effectors are remote controls for gene activation. Curr. Opin. Microbiol. 14, 47-53 (2011).
Zhang, F. et al. Efficient construction of sequence-specific TAL effectors for modulating mammalian transcription. Nat. Biotechnol. 29, 149-153 (2011).
Morbitzer, R., Romer, P., Boch, J. & Lahaye, T. Regulation of selected genome loci using de novo-engineered transcription activator-like effector (TALE)-type transcription factors. Proc. Natl. Acad. Sci. U.S.A. 107, 21617-21622 (2010).
Geissler, R. et al. Transcriptional activators of human genes with programmable DNA-specificity. PLoS One 6, e19509 (2011).
Mahfouz, M. et al. Targeted transcriptional repression using a chimeric TALE-SRDX repressor protein. Plant Mol. Biol. 78, 311-321 (2012).
Blount, B. A., Weenink, T., Vasylechko, S. & Ellis, T. Rational Diversification of a Promoter Providing Fine-Tuned Expression and Orthogonal Regulation for Synthetic Biology. PLoS One 7, e33279 (2012).
Mahfouz, M. M. et al. De novo-engineered transcription activator-like effector (TALE) hybrid nuclease with novel DNA binding specificity creates double-strand breaks. Proc. Natl. Acad. Sci. U.S.A. 108, 2623-2628 (2011).
Li, T. et al. TAL nucleases (TALNs): hybrid proteins composed of TAL effectors and FokI DNA-cleavage domain. Nucleic Acids Res. 39, 359-372 (2010).
Yu, Y. et al. Colonization of rice leaf blades by an African strain of Xanthomonas oryzae pv. oryzae depends on a new TAL effector which induces the rice nodulin-3 Os11N3 gene. Molecular Plant-Microbe Interactions 24, 1102-1113 (2011).
Römer, P. et al. Recognition of AvrBs3-Like Proteins Is Mediated by Specific Binding to Promoters of Matching Pepper Bs3 Alleles. Plant Physiology (Rockville) 150, 1697-1712 (2009).
Briggs, A. W. et al. Iterative capped assembly: rapid and scalable synthesis of repeat-module DNA such as TAL effectors from individual monomers. Nucleic Acids Res. (2012).
Sun, N., Liang, J., Abil, Z. & Zhao, H. Optimized TAL effector nucleases (TALENs) for use in treatment of sickle cell disease. Molecular BioSystems (2012).
Mak, A. N.-S., Bradley, P., Cernadas, R. A., Bogdanove, A. J. & Stoddard, B. L. The crystal structure of TAL effector PthXo1 bound to its DNA target. Science 335, 716-719 (2012).
Deng, D. et al. Structural Basis for Sequence-Specific Recognition of DNA by TAL Effectors. Science 335, 720-723 (2012).
Genin, S. & Denny, T. P. Pathogenomics of the Ralstonia solanacearum Species Complex. Annual Review of Phytopathology 50, 67-89 (2012).
Cunnac, S., Occhialini, A., Barberis, P., Boucher, C. & Genin, S. Inventory and functional analysis of the large Hrp regulon in Ralstonia solanacearum: identification of novel effector proteins translocated to plant host cells through the type III secretion system. Molecular Microbiology 53, 115-128 (2004).
Heuer, H., Yin, Y.-N., Xue, Q.-Y., Smalla, K. & Guo, J.-H. Repeat Domain Diversity of avrBs3-Like Genes in Ralstonia solanacearum Strains and Association with Host Preferences in the Field. Applied and Environmental Microbiology 73, 4379-4384 (2007).
Mukaihara, T., Tamura, N., Murata, Y. & Iwabuchi, M. Genetic screening of Hrp type III-related pathogenicity genes controlled by the HrpB transcriptional activator in Ralstonia solanacearum. Molecular Microbiology 54, 863-875 (2004).
Mukaihara, T., Tamura, N. & Iwabuchi, M. Genome-Wide Identification of a Large Repertoire of Ralstonia solanacearum Type III Effector Proteins by a New Functional Screen. Molecular Plant-Microbe Interactions 23, 251-262 (2010).
Salanoubat, M. et al. Genome sequence of the plant pathogen Ralstonia solanacearum. Nature 415, 497-502 (2002).
Verdier, V. et al. Transcription activator-like (TAL) effectors targeting OsSWEET genes enhance virulence on diverse rice (Oryza sativa) varieties when expressed individually in a TAL effector-deficient strain of Xanthomonas oryzae. New Phytol. 196, 1197-1207 (2012).
Nakagawa, T. et al. Development of series of gateway binary vectors, pGWBs, for realizing efficient construction of fusion genes for plant transformation. Journal of Bioscience and Bioengineering 104, 34-41 (2007).
Cermak, T. et al. Efficient design and assembly of custom TALEN and other TAL effector-based constructs for DNA targeting. Nucleic Acids Res. 39, e82 (2011).
Larkin, M. A. et al. Clustal W and Clustal X version 2.0. Bioinformatics 23, 2947-2948 (2007).
Goujon, M. et al. A new bioinformatics analysis tools framework at EMBL-EBI. Nucleic Acids Res. 38, W695-W699 (2010).
Buchan, D. W. A. et al. Protein annotation and modelling servers at University College London. Nucleic Acids Res. 38, W563-W568 (2010).

The contents of any patents, patent applications, and references cited throughout this specification are hereby incorporated by reference in their entireties.

Those skilled in the art will recognize, or be able to ascertain using no more than routine experimentation, many equivalents to the specific embodiments of the invention described herein. Such equivalents are intended to be encompassed by the following claims.

What is claimed is:

1. A method for modifying the genetic material of a cell, comprising:
(a) providing a cell containing a target DNA sequence; and
(b) introducing a transcription activator-like (TAL) effector-DNA modifying enzyme (TALEN) into the cell, the TALEN comprising:
(i) a DNA modifying enzyme domain that can modify double stranded DNA, and
(ii) a TAL effector domain comprising a plurality of TAL effector repeat sequences that, in combination, bind to a specific nucleotide sequence in the target DNA sequence, and further wherein said effector repeat sequences are such that the target DNA sequence does not require a 5′ thymine residue, and/or wherein said activity of TAL effector is increased.

2. The method of claim 1 wherein the DNA modifying enzyme is selected from the group comprising endonucelases, trascriptional activators, transcriptional repressors, dioxygenases, and methylases.

3. The method of claim 1, further comprising providing to the cell a nucleic acid comprising a sequence homologous to at least a portion of the target DNA sequence, such that homologous recombination occurs between the target DNA sequence and the nucleic acid.

4. The method of claim 1, wherein the cell is a eukaryotic cell.

5. The method of claim 1, wherein the cell is a mammalian cell.

6. The method of claim 1, wherein the cell is a plant cell.

7. The method of claim 1, wherein the cell is a prokaryotic cell.

8. The method of claim 1, wherein the target DNA is chromosomal DNA.

9. The method of claim 1, wherein the introducing comprises transfecting the cell with a vector encoding the TAL effector-DNA modifying enzyme.

10. The method of claim 1, wherein the introducing comprises mechanically injecting the TAL effector-DNA modifying enzyme into the cell as a protein.

11. The method of claim 1, wherein the introducing comprises delivering the TAL effector-DNA modifying enzyme into the cell as a protein by means of the bacterial type III secretion system.

12. The method of claim 1, wherein the introducing comprises introducing the TAL effector-DNA modifying enzyme into the cell as a protein by electroporation.

13. The method of claim 1, wherein the DNA modifying enzyme domain is an endonuclease.

14. The method of claim 13, wherein the endonuclease is a type II restriction endonuclease.

15. The method of claim 14, wherein the type II restriction endonuclease is FokI.

16. The method of claim 1, wherein the TAL effector domain that binds to a specific nucleotide sequence within the target DNA comprises 10 or more DNA binding repeats, more preferably 15 or more DNA binding repeats, as well as a 0^{th} and −1 repeat which do not require a thymine residue in the target DNA sequence.

17. The method of claim 16, wherein each DNA binding repeat comprises a repeat variable-diresidue (RVD) that determines recognition of a base pair in the target DNA sequence, wherein each DNA binding repeat is responsible for recognizing one base pair in the target DNA sequence, and wherein the RVD comprises one or more of: HD for recognizing C; NG for recognizing T; NI for recognizing A; NN for recognizing G or A; NS for recognizing A or C or G or T; N* for recognizing C or T, wherein * represents a gap in the second position of the RVD; HG for recognizing T; H* for recognizing T, wherein * represents a gap in the second position of the RVD; IG for recognizing T; NK for recognizing G; HA for recognizing C; ND for recognizing C; HI for recognizing C; HN for recognizing G; NA for recognizing G; SN for recognizing G or A; and YG for recognizing T.

18. The method of claim 17, wherein each DNA binding repeat comprises a RVD that determines recognition of a base pair in the target DNA sequence, wherein each DNA binding repeat is responsible for recognizing one base pair in the target DNA sequence, and wherein the RVD comprises one or more of: HA for recognizing C; ND for recognizing C; HI for recognizing C; FIN for recognizing G; NA for recognizing G; SN for recognizing G or A; YG for recognizing T; and NK for recognizing G; and one or more of: HD for recognizing C; NG for recognizing T; NI for recognizing A; NN for recognizing G or A; NS for recognizing A or C or G or T; N* for recognizing C or T, wherein * represents a gap in the second position of the RVD; HG for recognizing T; H* for recognizing T, wherein * represents a gap in the second position of the RVD; and IG for recognizing T.

19. The method of claim 1 wherein said TAL effector is a PthXo1 effector.

20. The method of claim 19 within said PthXo1 effector has a replacement of tryptophan at position 232 with another amino acid.

21. The method of claim 20 wherein said amino acid is selected from the group consisting of: glutamine, threonine, proline, arginine or asparagine.

22. The method of claim 1 wherein W*, QW, or the WS in the −1 repeat is replaced with one or more of NG, HD, NI, NN.

23. The method of claim 1 wherein the R*, KR, or RG of the 0^{th} repeat is replaced with NG, HD, NI, NN.

24. The method of claim 1 wherein the TAL effector domain includes part of a TAL effector-like protein of Ralstonia solanacearum.

25. A TAL effector comprising an endonuclease domain and a TAL effector DNA binding domain (TALEN) specific for a target DNA, wherein the DNA binding domain comprises a plurality of DNA binding repeats, each repeat comprising a RVD that determines recognition of a base pair in the target DNA, wherein each DNA binding repeat is responsible for recognizing one base pair in the target DNA, and wherein the TALEN comprises a RVD at the 0^{th} or −1th position that eliminates the need for a thymine 5′ to the target DNA binding domain.

26. The TAL effector of claim 25 wherein the TALEN comprises an amino acid substitution for the tryptopha at position 232 that eliminates the need for a thymine 5′ to the target DNA binding domain.

27. The TALEN of claim 25, wherein the TALEN comprises one or more of the following RVDs: HA for recognizing C; ND for recognizing C; HI for recognizing C; HN for recognizing G; NA for recognizing G; SN for recognizing G or A; YG for recognizing T; and NK for recognizing G, and one or more of: HD for recognizing C; NG for recognizing T; NI for recognizing A; NN for recognizing G or A; NS for recognizing A or C or G or T; N* for recognizing C or T; HG for recognizing T; H* for recognizing T; and IG for recognizing T.

28. The TALEN of claim 25, wherein the endonuclease domain is from a type II restriction endonuclease.

29. The TALEN of claim 28, wherein the type II restriction endonuclease is FokI.

30. The TALEN of claim 25 wherein the TAL effector DNA binding domain is a Xanthomonas TAL effector.

31. TALEN of claim 25 wherein the TAL effector DNA binding domain is a PthXo1 TAL effector.

32. The TALEN of claim 25 wherein the TAL effector DNA binding domain is a AcrXa7 TAL effector.

33. The TALEN of claim 25 wherein the TAL effector DNA binding domain is a PthXo3 TAL effector.

34. The TALEN of claim 25 wherein the TAL effector DNA binding domain is a PthXo2 TAL effector.

35. The TALEN of claim 25 wherein the TAL effector DNA binding domain is from a TAL effector-like protein of Ralstonia solanacearum.

36. A method for generating a modified transcription activator-like (TAL) effector-DNA modifying enzyme (TALEN) comprising an endonuclease domain and a TAL effector DNA binding domain, comprising altering the −1 and/or the 0^{th} repeats of such that said TAL effector DNA binding domain targets a nucleotide sequence with a cytosine, adenine, guanine, or thymine at the 5′ position.

37. The method of claim 34 wherein the TALEN targets a sequence with a 5′ cytosine, wherein said alteration comprises one of the following modifications is made in the 0^{th} repeat:
KR*GG to SHDGG
KR*GG to KHDGG
KRGG to HDGG
KRGG to KHDG
and/or wherein one of the following modifications is made in the −1 repeat:
QWS to QAS
QW*S to QHDS
QWS to HDS
QWS to QHD.

38. The method of claim 34 wherein the TALEN targets a sequence with a 5′ adenine, wherein said alteration comprises one of the following modifications is made in the 0^{th} repeat:
KR*GG to SNIGG
KR*GG to KNIGG
KRGG to NIGG
KRGG to KHDG
and/or wherein one of the following modifications is made in the −1 repeat:
QWS to QAS
QW*S to QNIS
QWS to NIS
QWS to QNI.

39. The method of claim 34 wherein the TALEN targets a sequence with a 5′ guanine or adenine, wherein said alteration comprises one of the following modifications is made in the 0^{th} repeat:
KR*GG to SNNGG
KR*GG to KNNGG
KRGG to NNGG
KRGG to KNNG
and/or wherein one of the following modifications is made in the −1 repeat:
QWS to QAS
QW*S to QNNS
QWS to NNS
QWS to QNN.

40. The method of claim 34 wherein the TALEN targets a sequence with a 5′ T, wherein said alteration comprises one of the following modifications is made in the 0^{th} repeat:
KR*GG to SNGGG
KR*GG to KNGGG
KRGG to NGGG
KRGG to KNGG
and/or wherein one of the following modifications is made in the −1 repeat:
QWS to QAS
QW*S to QNGS
QWS to NGS
QWS to QNG.

41. A nucleic acid sequence which encodes a TAL effector fusion protein comprising an endonuclease domain and a TAL effector DNA binding domain specific for a target DNA, which has been designed to interact with and cleave a target sequence with any nucleotide at the 5′ position including one or more of:
(a) SEQ NO:1, 78, 80, or 82,
(b) a nucleic acid sequence which encodes SEQ ID NO:2, 77, 79, or 81 and its conservatively modified variants;
(c) a nucleic acid sequence which hybridizes under conditions of high stringency to sequences in (a) or (b);
(d) a nucleic acid sequence which has 90% or greater sequence similarity to (a) or (b).

42. An expression construct including the nucleic acid sequence of claim 39 operably linked to a promoter sequence capable of directing expression in a cell.

43. A vector incorporating the expression construct of claim 40.

44. A cell including the vector of claim 41.

45. The cell of claim 42, wherein the cell is a eukaryotic cell.

46. The cell of claim 42, wherein the cell is a mammalian cell.

47. The cell of claim 42, wherein the cell is a plant cell.

48. The cell of claim 42, wherein the cell is a prokaryotic cell.

49. The nucleic acid of claim 39, wherein the target DNA is chromosomal DNA.
